(19) United States
(12) Patent Application Publication
(10) Pub. No.: US 2008/0293771 A1
(43) Pub. Date: Nov. 27, 2008

(54) AZACYCLYLBENZAMIDE DERIVATIVES AS HISTAMINE-3 ANTAGONISTS

(75) Inventors: Dahui Zhou, East Brunswick, NJ (US); Jean Yi-Ching Sze, Monmouth Junction, NJ (US); Jonathan Laird Gross, Cranbury, NJ (US); Albert Jean Robichaud, Ringoes, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/126,232

(22) Filed: May 23, 2008

Related U.S. Application Data
(60) Provisional application No. 60/931,519, filed on May 24, 2007.

(51) Int. Cl. A61K 31/454; C07D 403/12; A61K 31/4184; A61P 25/28; A61P 25/18; C07K 14/705; C07D 401/12

(52) U.S. Cl. 514/322; 548/306.1; 514/394; 546/199; 530/350

(57) ABSTRACT

The present invention provides a compound of formula I and the use thereof for the treatment of a central nervous system disorder related to or affected by the histamine-3 receptor

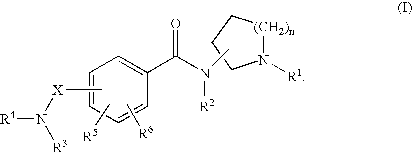

### CROSS-REFERENCE TO RELATED APPLICATIONS

This application claims the benefit under 35 U.S.C. §119(e) to co-pending U.S. provisional application No. 60/931519, filed May 24, 2007, which is hereby incorporated by reference in its entirety.

### FIELD OF THE INVENTION

The current invention relates to azacyclylbenzamide compounds, their use in modulation of the histamine-3 (H₃) receptor and treatment of a variety of central nervous system disorders related to or affected by the H₃ receptor. The invention also provides methods of synthesis and pharmaceutical compositions comprising the azacyclylbenzamide compounds.

### BACKGROUND OF THE INVENTION

The histamine-3 (H₃) receptor is one of four histamine receptor subtypes (H₁-H₄), all of which are members of the G-protein-coupled receptor (GCPR) superfamily. The H₃ receptor is predominantly expressed in the central nervous system. In the brain, it is located in regions associated with learning and memory such as the cerebral cortex, hippocampus and striatum.

The H₃ receptor acts as both auto- and hetero-receptor to regulate the release of histamine and other neurotransmitters. Within the cortex, the H₃ receptor appears to directly modify GABA release from cortical interneurons. Antagonism of the H₃ receptor produces a decrease in GABA release and disinhibition of the cortical cholinergic system, resulting in increased acetylcholine levels (Bacciottini, L. et al, Behavioral Brain Research, 124, 2001, 183-194). In addition to direct regulation of cholinergic neurotransmission, the H₃ receptor has been shown to modulate the release of dopamine, serotonin and norepinephrine (Leurs, R., et al, Trends in Pharmacological Sciences, 19, 1998, 177-183). Thus, H₃ receptor blockade is able to elevate concentrations of a number of neurotransmitters, including: histamine, acetylcholine, dopamine, serotonin, norepinephrine, and glutamate, and thus offers a means for targeting cognitive processes, which often rely on the integration of multiple neurotransmitter systems.

H₃ agonists have been reported to impair memory in various tasks, such as object recognition, passive avoidance (Blandina, P., et al, British Journal of Pharmacology, 119(8), 1996, 1656-1664) and social olfactory memory (Prast, H., et al, 734, 1996, 316-318), whereas H₃ antagonists have been reported to rescue impairments produced pharmacologically or genetically. Miyazaki, S., et al, Life Sciences, 61, 1997, 355-361; Meguro, K., et al, Pharmacology, Biochemistry and Behavior, 50, 1995, 321-325; Fox, G. B., et. al, Behavioral Brain Research, 131, 2002, 151-161; and Komater, V. A., et al, Psychopharmacology, 167, 2003, 363-372.

H₃ receptors are targets for the control of arousal and vigilance as well as for the treatment of sleep disorders because they colocalize with histaminergic neurons in brain regions that regulate the sleep-wake cycle and they modulate histamine release and levels in the CNS. Passani et al. Trends Pharmacol. Sci. 25, 618-25, 2004. The administration of selective H₃ receptor agonists, such as R-α-methylhistamine, increases sleep time and slow wave sleep in cats and rodents and produces sedation in the guinea pig, whereas H₃ antagonists such as thioperamide increase wakefulness in cats and rats and decrease slow wave sleep and REM sleep in rats. Monti et al. Eur. J. Pharmacol. 205, 283-287, 1991 and Esbenshade et al. Molecular Interventions 6:77-88, 2006.

Studies on memory consolidation and spatial memory impairments, which are particularly prevelant in AD and dimentia, have revealed that the H₃ antagonist thioperamide improves recall in a mouse model of premature senescence as well as in spontaneously hypertensive rat pups, and also prevents scopolamine-induced amnesia. Meguro et al. Pharmacol. Biochem. Behav. 50, 321-325, 1995 and Hancock et al. Expert Opin. Investig. Drugs 13, 1237-1248, 2004. Further, H₃ receptor knockout mice are insensitive to the effects of scopolamine in an inhibitory avoidance paradigm, supporting a role for H₃ receptor modulation of cholinergic function in memory acquisition. Toyota et al. Mol. Pharmacol. 62, 389-397, 2002.

Impairments in social recognition memory are apparent in AD, but may also be relevant to social cognitive impairment in schizophrenia and ADHD. Esbenshade et al. Molecular Interventions 6:77-88, 2006. Social recognition tests have been used to show that the administration of selective histaminergic agonists enhances social memory, whereas recall is disrupted by the inhibition of histamine synthesis. Prast et al. Brain Res. 734, 316-318, 1996. In particular, thioperamide as well as several other H₃ receptor antagonists have been attributed with pro-cognitive effects. Id. In working memory impairments, prevalent in AD, ADHD, and schizophrenia, thioperamide reverses scopolamine-induced deficits. Barbier et al. Br. J. Pharmacol. 143, 649-661, 2004 and Fox et al. J. Pharmacol. Exp. Ther. 305, 897-908, 2003. Thioperamide, ciproxifan, and GT-2331, all H₃ antagonists, are also efficacious in treating impulsivity associated with ADHD in spontaneous hypertensive rat pups. Fox et al. Behav. Brain Res. 131, 151-161, 2002.

The H₃ receptor is also involved in pathological processes in the 6-OHDA-lesioned rat brain, a well-characterized model of Parkinson's disease. Increased H₃ receptor mRNA expression and binding may, for example, modulate GABAergic neuronal activity in dopamine-depleted striatum. Anichtchik et al., European Journal of Neuroscience, 12 (11), 3823-3832 2000.

Methamphetamine-induced hyperlocomotor activity, a behaviorally relevant model for psychosis, can be attenuated by ciproxifan in mice (Morisset et al. J. Pharmacol. Exp. Ther. 300, 621-628, 2002), as well as by the antipsychotic drug risperidone and the H₃ receptor antagonist ABT-239. Fox et al. J. Pharmacol. Exp. Ther. 313, 176-190 (2005). H₃ antagonists, such as thioperamide, have also been shown to reduce cumulative food consumption, weight gain and are suggested to have antidepressant activity. Esbenshade et al. supra and Perez-Garcia et al. Psychopharmacologia, 142(2) 215-220. 1999.

Accordingly, there is significant neuroanatomical, neurochemical, pharmacological and behavioral data to support the use of H₃ receptor antagonists for improving cognitive performance in disease states such as neurodegeneration, cognitive impairment, Alzheimer's disease, Parkinson's disease, dementia, psychosis, depression, attention deficit disorder (ADD)/attention deficit hyperactivity disorder (ADHD), schizophrenia, obesity and sleep disorders.

Therefore, it is an object of this invention to provide compounds which are inhibitors of the H₃ receptor and are useful as therapeutic agents in the treatment of a variety of central nervous system disorders related to or affected by the H₃ receptor. It is another object of this invention to provide therapeutic methods and pharmaceutical compositions useful for the treatment of central nervous system disorders related to or affected by the H₃ receptor. It is a feature of this invention that the compounds provided may also be useful to further study and elucidate the H₃ receptor.

### SUMMARY OF THE INVENTION

The present invention provides an azacyclylbenzamide compound of formula I:

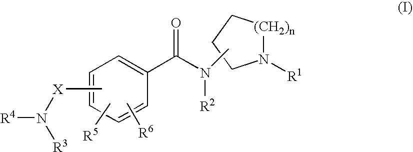

wherein
X is (CR⁷R⁸)ₘ, CO or SO₂;
m is 0 or 1;
n is 1, 2 or 3;
R¹ is H, C₁-C₆ alkyl, C₁-C₆ haloalkyl, C₃-C₁₀ cycloalkyl or a 3-10 membered cycloheteroalkyl each group optionally substituted;
R² is H or C₁-C₆ alkyl or C₃-C₁₀ cycloalkyl each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen or C₁-C₆ alkyl, C₃-C₁₀ cycloalkyl or C₁-C₆ alkoxy each optionally substituted; or R⁵ and R⁶ are taken together with the atoms to which they are attached to form an optionally substituted phenyl ring;
R⁷ and R⁸ are each independently H, halogen or C₁-C₆ alkyl or C₃-C₁₀ cycloalkyl each group optionally substituted; or

a stereoisomer, tautomer or pharmaceutically acceptable salt thereof.

In a more particular embodiment thereof, if R² is H or R³ and R⁴ are taken together to form a tricyclic aromatic ring system, then n is not 2.

The present invention also provides methods and compositions useful for the therapeutic treatment of central nervous system disorders related to or affected by the Histamine-3 receptor.

Another embodiment of the present invention provides use of a composition of any one of the embodiments described herein for the treatment of a central nervous system disorder related to or affected by the H₃ receptor. More particularly, the present invention provides for use of a compound of any one of the embodiments described herein for the manufacture of a medicament for the treatment of a central nervous system disorder related to or affected by the H₃ receptor.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

Alzheimer's disease (AD) is characterized by a progressive loss of memory and cognitive function and is the most common cause of dementia in the elderly. AD is believed to affect approximately 15-20 million people worldwide. The goal of treatment in AD, in addition to reversing the disease process, is to improve or at least slow the loss of memory and cognition and to maintain independent function in patients with mild to moderate disease. AD is characterized by numerous deficits in neurotransmitter function (Möller, H-J., European Neuropsychopharmacology, 9, 1999, S53-S59), further a postmortem study in humans suggests that a decrease in brain histamine levels may contribute to the cognitive decline associated with AD, directly or through the cholinergic system (Panula, P., et al, Neuroscience, 82, 1998, 993-997). Histamine-3 (H₃) receptor antagonists have been reported to rescue impairments produced pharmacologically or genetically (Miyazaki, S., et al, Life Sciences, 61, 1997, 355-361; Meguro, K., et al, Pharmacology, Biochemistry and Behavior, 50, 1995, 321-325; Fox, G. B., et. al, Behavioral Brain Research, 131, 2002, 151-161; and Komater, V. A., et al, Psychopharmacology, 167, 2003, 363-372). Neuroanatomical, neurochemical, pharmacological and behavioral data support the belief that H₃ receptor antagonists may improve cognitive performance in disease states such as mild cognitive impairment and Alzheimer's disease and may have therapeutic value in the treatment of attention deficit disorder (ADD)/attention deficit hyperactivity disorder (ADHD), schizophrenia, dementia, psychosis, depression, Parkinson's disease, obesity and sleep disorders. To that end, compounds which inhibit the H₃ receptor and act as H₃ antagonists are earnestly sought.

Surprisingly it has now been found that azacyclylbenzamide compounds of formula I demonstrate H₃ affinity along with significant sub-type selectivity and function as H₃ antagonists. Advantageously, said formula I compounds are effective therapeutic agents for the treatment of central nervous system (CNS) disorders associated with or affected by the H₃ receptor. Accordingly, the present invention provides an azacyclylbenzamide compound of formula I

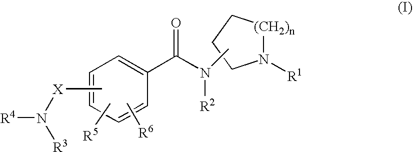

wherein
X is (CR⁷R⁸)ₘ, CO or SO₂;
m is 0 or 1;
n is 1, 2 or 3;
R¹ is an alkyl, haloalkyl, cycloalkyl or cycloheteroalkyl group each group optionally substituted;
R² is H or an alkyl or cycloalkyl group each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen or an alkyl, cycloalkyl or a C₁-C₆ alkoxy group each optionally substituted; or R⁵ and R⁶ are taken together with the atoms to which they are attached to form an optionally substituted phenyl ring;
R⁷ and R⁸ are each independently H, halogen or an alkyl or cycloalkyl group each group optionally substituted; or
   a stereoisomer thereof or a pharmaceutically acceptable salt thereof.

In a more particular embodiment thereof, R⁵ and R⁶ are both H.

Particular compounds of the invention include those compounds of formula I wherein n is 1 or 2. Another group of compounds is those of formula I compounds wherein X is (CR⁷R⁸)ₘ. Also preferred are those formula I compounds wherein R³ and R⁴ are taken together with the atom to which they are attached to form an optionally substituted benzimidazole, pyrazole, indazole or indole ring system.

More particular compounds of the invention are those compounds of formula I wherein R¹ is isopropyl or C₃-C₆cycloalkyl; X is (CR⁷R⁸)ₘ; and R⁷ and R⁸ are each independently H or CH₃. Another group of compounds are those compounds of formula I wherein n is 1 or 2; R¹ is isopropyl or C₃-C₆cycloalkyl; X is (CR⁷R⁸)ₘ; and R⁷ and R⁸ are each independently H or CH₃. A further group of compounds are those compounds of formula I wherein n is 1 or 2; R¹ is isopropyl or C₃-C₆ cycloalkyl; and R³ and R⁴ are taken together with the atom to which they are attached to form an optionally substituted benzimidazole, indazole, pyrazole or indole ring system.

In another embodiment of the compound of formula (I):
X is (CR⁷R⁸)ₘ, CO or SO₂;
m is 0 or 1;
n is 1, 2 or 3;
R¹ is H, C₁-C₆ alkyl, C₁-C₆ haloalkyl, C₃-C₁₀ cycloalkyl or a 3-10 membered cycloheteroalkyl each group optionally substituted;
R² is H or C₁-C₆ alkyl or C₃-C₁₀ cycloalkyl each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen or C₁-C₆ alkyl or C₃-C₁₀ cycloalkyl or C₁-C₆ alkoxy each optionally substituted; or R⁵ and R⁶ are taken together with the atoms to which they are attached to form an optionally substituted phenyl ring;
R⁷ and R⁸ are each independently H, halogen or C₁-C₆ alkyl or C₃-C₁₀ cycloalkyl each group optionally substituted; or

a stereoisomer, tautomer or pharmaceutically acceptable salt thereof;

provided that if R² is H or R³ and R⁴ are taken together to form a tricyclic aromatic ring system, then n is not 2.

In another embodiment, n is 1 or 2. In another embodiment, X is (CR⁷R⁸)ₘ. More particularly, wherein m is 0. Alternatively, m is 1 and R⁷ and R⁸ are both H.

In another more particular embodiment of the compound of formula I, R³ and R⁴ are taken together with the atom to which they are attached to form the structure of formula IA:

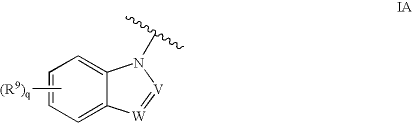

wherein,

q is 0, 1, 2 or 3;

V and W are independently N or CR¹⁰;

R⁹ is independently halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-7 membered heteroaryl or heterocyclyl group, or C₃-C₆ cycloalkyl, wherein each C₁-C₆ alkyl, C₁-C₆ acyl, C₁-C₆ alkoxy, C₆-C₁₀ aryl, 5-7 membered heteroaryl or heterocyclyl group, or C₃-C₆ cycloalkyl is substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, a 5-7 membered heterocyclyl or heteroaryl ring, —N(R^{a})ₜ, —C(O)R^{b}, —OR^{c} and —S(O)ₚR^{d};

R¹⁰ is independently H, halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-7 membered heteroaryl or heterocyclyl group, or C₃-C₆ cycloalkyl, wherein each C₁-C₆ alkyl, C₁-C₆ acyl, C₁-C₆ alkoxy, C₆-C₁₀ aryl, 5-7 membered heteroaryl or heterocyclyl group, or C₃-C₆ cycloalkyl is substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, a 5-7 membered heterocyclyl or heteroaryl ring, —N(R^{a})ₜ, —C(O)R^{b}, —OR^{c} and —S(O)ₚR^{d};

each R^{a} is independently H, C₁-C₄ alkyl, —CHO, —C(O)(C₁-C₄ alkyl) or —CO₂(C₁-C₄ alkyl);

each R^{b} is independently H, —OH, —O(C₁-C₄), C₁-C₄ alkyl, —NH₂, —NH(C₁-C₄ alkyl) or —N(C₁-C₄ alkyl)₂;

each R^{c} is independently H, C₁-C₄ alkyl, C₁-C₄ haloalkyl, —CHO or —C(O)(C₁-C₄ alkyl);

each R^{d} is independently H, C₁-C₄ alkyl or —OH; and

each p is independently 0, 1 or 2.

In another embodiment of the structure of formula IA, q is 0. In another embodiment, W is N and V is CR¹⁰. More particularly, R¹⁰ is C₁-C₃ alkyl, more particular still, methyl. In another embodiment, V is N and W is CR¹⁰. More particularly, R¹⁰ is H. In another embodiment, R² is methyl or ethyl.

In another embodiment of the compound of formula I, R³ and R⁴ are taken together with the atom to which they are attached to form an optionally substituted pyrazole, benzimidazole, indazole or indole ring system. In another embodiment, R¹ is C₁-C₆ alkyl or C₃-C₆ cycloalkyl. In another embodiment, R¹ is methyl, ethyl, propyl or isopropyl.

In another embodiment, R³ and R⁴ are taken together with the atom to which they are attached to form the structure of formula IB:

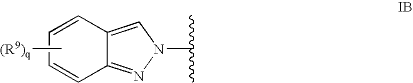

wherein,

q is 0, 1, 2 or 3; and

R⁹ is independently halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-7 membered heteroaryl or heterocyclyl group, or C₃-C₆ cycloalkyl, wherein each C₁-C₆ alkyl, C₁-C₆ acyl, C₁-C₆ alkoxy, C₆-C₁₀ aryl, 5-7 membered heteroaryl or heterocyclyl group, or C₃-C₆ cycloalkyl is substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, a 5-7 membered heterocyclyl or heteroaryl ring, —N(R^{a})ₜ, —C(O)R^{b}, —OR^{c} and —S(O)ₚR^{d};

each R^{a} is independently H, C₁-C₄ alkyl, —CHO, —C(O)(C₁-C₄ alkyl) or —CO₂(C₁-C₄ alkyl);

each R^{b} is independently H, —OH, —O(C₁-C₄), C₁-C₄ alkyl, —NH₂, —NH(C₁-C₄ alkyl) or —N(C₁-C₄ alkyl)₂;

each R^{c} is independently H, C₁-C₄ alkyl, C₁-C₄ haloalkyl, —CHO or —C(O)(C₁-C₄ alkyl);

each R^{d} is independently H, C₁-C₄ alkyl or —OH; and

each p is independently 0, 1 or 2.

In another embodiment, q is 0.

In another embodiment, R¹ is methyl, ethyl, propyl, isopropyl, cyclopropylmethyl, cyclopentylmethyl, cyclohexylmethyl, cyclobutyl, cyclopentyl, tetrahydropyran-4-yl, bicyclo[2.2.1]hept-2-yl, or adamantan-2-yl.

Another aspect of the invention provides a compound of formula:

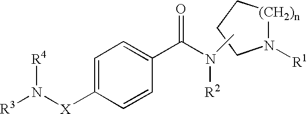

wherein
X is (CH₂)ₘ;
m is 0 or 1;
n is 1 or 2;
R¹ is C₁-C₆ alkyl or C₃-C₆ cycloalkyl each group optionally substituted;
R² is H or C₁-C₆ alkyl; and
R³ and R⁴ are taken together with the atom to which they are attached to form an optionally substituted monocyclic 5-membered aromatic ring system optionally containing one or two additional heteroatoms selected from N, O or S or an optionally substituted fused bicyclic aromatic ring system optionally containing one to three additional heteroatoms selected from N, O or S; or

a stereoisomer, tautomer or pharmaceutically acceptable salt thereof.

Another aspect of the invention provides a compound of formula:

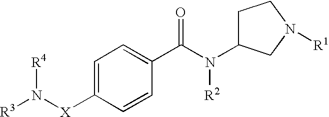

wherein
X is (CH₂)ₘ;
m is 0 or 1;
R¹ is C₁-C₆ alkyl or C₃-C₆ cycloalkyl each group optionally substituted;
R² is H or C₁-C₆ alkyl; and
R³ and R⁴ are taken together with the atom to which they are attached to form an optionally substituted monocyclic 5-membered aromatic ring system optionally containing one or two additional heteroatoms selected from N, O or S or an optionally substituted fused bicyclic aromatic ring system optionally containing one to three additional heteroatoms selected from N, O or S; or

a stereoisomer, tautomer or pharmaceutically acceptable salt thereof.

Another aspect of the invention provides a compound of formula:

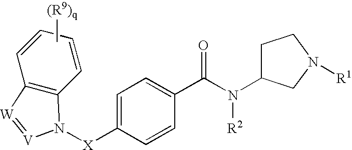

wherein

X is (CH₂)ₘ;

m is 0 or 1;

R¹ is C₁-C₆ alkyl or C₃-C₆ cycloalkyl each group optionally substituted;

R² is H or C₁-C₆ alkyl;

q is 0, 1, 2 or 3;

V and W are independently N or CR¹⁰;

R⁹ is independently halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-7 membered heteroaryl or heterocyclyl group, or C₃-C₆ cycloalkyl, wherein each C₁-C₆ alkyl, C₁-C₆ acyl, C₁-C₆ alkoxy, C₆-C₁₀ aryl, 5-7 membered heteroaryl or heterocyclyl group, or C₃-C₆ cycloalkyl is substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, a 5-7 membered heterocyclyl or heteroaryl ring, —N(R^{a})ₜ, —C(O)R^{b}, —OR^{c} and —S(O)ₚR^{d};

R¹⁰ is independently H, halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-7 membered heteroaryl or heterocyclyl group, or C₃-C₆ cycloalkyl, wherein each C₁-C₆ alkyl, C₁-C₆ acyl, C₁-C₆ alkoxy, C₆-C₁₀ aryl, 5-7 membered heteroaryl or heterocyclyl group, or C₃-C₆ cycloalkyl is substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, a 5-7 membered heterocyclyl or heteroaryl ring, —N(R^{a})ₜ, —C(O)R^{b}, —OR^{c} and —S(O)ₚR^{d};

each R^{a} is independently H, C₁-C₄ alkyl, —CHO, —C(O)(C₁-C₄ alkyl) or —CO₂(C₁-C₄ alkyl);

each R^{b} is independently H, —OH, —O(C₁-C₄), C₁-C₄ alkyl, —NH₂, —NH(C₁-C₄ alkyl) or —N(C₁-C₄ alkyl)₂;

each R^{c} is independently H, C₁-C₄ alkyl, C₁-C₄ haloalkyl, —CHO or —C(O)(C₁-C₄ alkyl);

each R^{d} is independently H, C₁-C₄ alkyl or —OH; and

each p is independently 0, 1 or 2; or

a stereoisomer, tautomer or pharmaceutically acceptable salt thereof.

In a more particular embodiment m is 0; or R³ and R⁴ combine to form the structure of formula IA or IB; or R¹ is methyl, ethyl, propyl, isopropyl, cyclopropylmethyl, cyclopentylmethyl, cyclohexylmethyl, cyclobutyl, cyclopentyl, tetrahydropyran-4-yl, bicyclo[2.2.1]hept-2-yl, or adamantan-2-yl; or R² is C₁-C₆ alkyl, preferably methyl or ethyl.

In another embodiment, q is 1 and R⁹ is methoxy.

An exemplary embodiment of the present invention provides a compound selected from the group consisting essentially of:
N-methyl-4-(2-methyl-1H-benzimidazol-1-yl)-N-[(3R)-pyrrolidin-3-yl]benzamide
N-methyl-4-(2-methyl-1H-benzimidazol-1-yl)-N-[(3S)-pyrrolidin-3-yl]benzamide
N-methyl-4-(2-methyl-1H-benzimidazol-1-yl)-N-piperidin-4-ylbenzamide
N-methyl-4-[(2-methyl-1H-benzimidazol-1-yl)methyl]-N-[(3R)-pyrrolidin-3-yl]benzamide
4-(1H-benzimidazol-1-ylmethyl)-N-methyl-N-[(3R)-pyrrolidin-3-yl]benzamide
N-methyl-4-[(2-methyl-1H-benzimidazol-1-yl)methyl]-N-piperidin-4-ylbenzamide
4-(1H-benzimidazol-1-ylmethyl)-N-methyl-N-piperidin-4-ylbenzamide
4-(5-cyano-2-methyl-1H-benzimidazol-1-yl)-N-methyl-N-[(3R)-pyrrolidin-3-yl]benzamide
4-(1H-indazol-1-yl)-N-methyl-N-[(3R)-pyrrolidin-3-yl]benzamide
4-(2H-indazol-2-yl)-N-methyl-N-[(3R)-pyrrolidin-3-yl]benzamide
4-(2H-indazol-2-yl)-N-methyl-N-piperidin-4-ylbenzamide
4-(1H-indazol-1-yl)-N-methyl-N-piperidin-4-ylbenzamide
4-(1H-indazol-1-ylmethyl)-N-methyl-N-[(3R)-pyrrolidin-3-yl]benzamide
4-(1H-indazol-1-ylmethyl)-N-methyl-N-piperidin-4-ylbenzamide
N-methyl-4-(1H-pyrazol-1-yl)-N-[(3R)-pyrrolidin-3-yl]benzamide
N-methyl-N-piperidin-4-yl-4-(1H-pyrazol-1-yl)benzamide
N-methyl-4-(1H-pyrazol-1-ylmethyl)-N-[(3R)-pyrrolidin-3-yl]benzamide
N-methyl-N-piperidin-4-yl-4-(1H-pyrazol-1-ylmethyl)benzamide
4-(2-methyl-1H-benzimidazol-1-yl)-N-[(3R)-pyrrolidin-3-yl]benzamide
N-ethyl-4-(2-methyl-1H-benzimidazol-1-yl)-N-[(3R)-pyrrolidin-3-yl]benzamide
N-[(3R)-1-isobutylpyrrolidin-3-yl]-N-methyl-4-(2-methyl-1H-benzimidazol-1-yl)benzamide
N-[(3R)-1-cyclohexylpyrrolidin-3-yl]-N-methyl-4-(2-methyl-1H-benzimidazol-1-yl)benzamide
N-[(3R)-1-ethylpyrrolidin-3-yl]-N-methyl-4-(2-methyl-1H-benzimidazol-1-yl)benzamide
N-methyl-4-(2-methyl-1H-benzimidazol-1-yl)-N-[(3R)-1-propylpyrrolidin-3-yl]benzamide
N-[(3R)-1-(cyclopropylmethyl)pyrrolidin-3-yl]-N-methyl-4-(2-methyl-1H-benzimidazol-1-yl)benzamide
N-[(3R)-1-(cyclopentylmethyl)pyrrolidin-3-yl]-N-methyl-4-(2-methyl-1H-benzimidazol-1-yl)benzamide
N-[(3R)-1-(cyclohexylmethyl)pyrrolidin-3-yl]-N-methyl-4-(2-methyl-1H-benzimidazol-1-yl)benzamide
N-methyl-4-(2-methyl-1H-benzimidazol-1-yl)-N-[(3R)-1-methylpyrrolidin-3-yl]benzamide
N-[(3R)-1-isopropylpyrrolidin-3-yl]-N-methyl-4-(2-methyl-1H-benzimidazol-1-yl)benzamide
N-[(3R)-1-cyclobutylpyrrolidin-3-yl]-N-methyl-4-(2-methyl-1H-benzimidazol-1-yl)benzamide
N-[(3R)-1-cyclopentylpyrrolidin-3-yl]-N-methyl-4-(2-methyl-1H-benzimidazol-1-yl)benzamide
N-[(3R)-1-cycloheptylpyrrolidin-3-yl]-N-methyl-4-(2-methyl-1H-benzimidazol-1-yl)benzamide
N-methyl-4-(2-methyl-1H-benzimidazol-1-yl)-N-[(3R)-1-(tetrahydro-2H-pyran-4-yl)pyrrolidin-3-yl]benzamide
N-[(3R)-1-bicyclo[2.2.1]hept-2-ylpyrrolidin-3-yl]-N-methyl-4-(2-methyl-1H-benzimidazol-1-yl)benzamide
N-[(3R)-1-adamantan-2-ylpyrrolidin-3-yl]-N-methyl-4-(2-methyl-1H-benzimidazol-1-yl)benzamide
N-[(3S)-1-isopropylpyrrolidin-3-yl]-N-methyl-4-(2-methyl-1H-benzimidazol-1-yl)benzamide
N-[(3S)-1-cyclobutylpyrrolidin-3-yl]-N-methyl-4-(2-methyl-1H-benzimidazol-1-yl)benzamide
N-[(3S)-1-cyclopentylpyrrolidin-3-yl]-N-methyl-4-(2-methyl-1H-benzimidazol-1-yl)benzamide
N-[(3S)-1-cyclohexylpyrrolidin-3-yl]-N-methyl-4-(2-methyl-1H-benzimidazol-1-yl)benzamide
N-methyl-4-(2-methyl-1H-benzimidazol-1-yl)-N-[(3S)-1-(3-methylcyclopentyl)pyrrolidin-3-yl]benzamide
N-methyl-4-(2-methyl-1H-benzimidazol-1-yl)-N-{(3S)-1-[(3R)-3-methylcyclopentyl]pyrrolidin-3-yl}benzamide
N-methyl-4-(2-methyl-1H-benzimidazol-1-yl)-N-[(3S)-1-(2-methylcyclohexyl)pyrrolidin-3-yl]benzamide
N-methyl-4-(2-methyl-1H-benzimidazol-1-yl)-N-{(3S)-1-[(3R)-3-methylcyclohexyl]pyrrolidin-3-yl}benzamide
N-methyl-4-(2-methyl-1H-benzimidazol-1-yl)-N-[(3S)-1-(3-methylcyclohexyl)pyrrolidin-3-yl]benzamide
N-[(3S)-1-(cyclopropylmethyl)pyrrolidin-3-yl]-N-methyl-4-(2-methyl-1H-benzimidazol-1-yl)benzamide
N-(1-isopropylpiperidin-4-yl)-N-methyl-4-(2-methyl-1H-benzimidazol-1-yl)benzamide
N-(1-cyclopentylpiperidin-4-yl)-N-methyl-4-(2-methyl-1H-benzimidazol-1-yl)benzamide
N-(1-cyclohexylpiperidin-4-yl)-N-methyl-4-(2-methyl-1H-benzimidazol-1-yl)benzamide
N-(1-cyclobutylpiperidin-4-yl)-N-methyl-4-(2-methyl-1H-benzimidazol-1-yl)benzamide
N-[(3R)-1-isopropylpyrrolidin-3-yl]-N-methyl-4-[(2-methyl-1H-benzimidazol-1-yl)methyl]benzamide
N-[(3R)-1-cyclobutylpyrrolidin-3-yl]-N-methyl-4-[(2-methyl-1H-benzimidazol-1-yl)methyl]benzamide
N-[(3R)-1-cyclopentylpyrrolidin-3-yl]-N-methyl-4-[(2-methyl-1H-benzimidazol-1-yl)methyl]benzamide
N-[(3R)-1-cyclohexylpyrrolidin-3-yl]-N-methyl-4-[(2-methyl-1H-benzimidazol-1-yl)methyl]benzamide
4-(1H-benzimidazol-1-ylmethyl)-N-[(3R)-1-isopropylpyrrolidin-3-yl]-N-methylbenzamide
4-(1H-benzimidazol-1-ylmethyl)-N-[(3R)-1-cyclobutylpyrrolidin-3-yl]-N-methylbenzamide
4-(1H-benzimidazol-1-ylmethyl)-N-[(3R)-1-cyclopentylpyrrolidin-3-yl]-N-methylbenzamide
N-(1-isopropylpiperidin-4-yl)-N-methyl-4-[(2-methyl-1H-benzimidazol-1-yl)methyl]benzamide
N-(1-cyclobutylpiperidin-4-yl)-N-methyl-4-[(2-methyl-1H-benzimidazol-1-yl)methyl]benzamide
N-(1-cyclopentylpiperidin-4-yl)-N-methyl-4-[(2-methyl-1H-benzimidazol-1-yl)methyl]benzamide
4-(1H-benzimidazol-1-ylmethyl)-N-(1-isopropylpiperidin-4-yl)-N-methylbenzamide
4-(1H-benzimidazol-1-ylmethyl)-N-(1-cyclobutylpiperidin-4-yl)-N-methylbenzamide
4-(1H-benzimidazol-1-ylmethyl)-N-(1-cyclopentylpiperidin-4-yl)-N-methylbenzamide
4-(5-cyano-2-methyl-1H-benzimidazol-1-yl)-N-[(3R)-1-isopropylpyrrolidin-3-yl]-N-methylbenzamide
4-(5-cyano-2-methyl-1H-benzimidazol-1-yl)-N-[(3R)-1-cyclobutylpyrrolidin-3-yl]-N-methylbenzamide
4-(5-cyano-2-methyl-1H-benzimidazol-1-yl)-N-[(3R)-1-cyclopentylpyrrolidin-3-yl]-N-methylbenzamide
4-(2H-indazol-2-yl)-N-[(3R)-1-isopropylpyrrolidin-3-yl]-N-methylbenzamide
N-[(3R)-1-cyclobutylpyrrolidin-3-yl]-4-(2H-indazol-2-yl)-N-methylbenzamide
N-[(3R)-1-cyclopentylpyrrolidin-3-yl]-4-(2H-indazol-2-yl)-N-methylbenzamide
4-(1H-indazol-1-yl)-N-[(3R)-1-isopropylpyrrolidin-3-yl]-N-methylbenzamide
N-[(3R)-1-cyclobutylpyrrolidin-3-yl]-4-(1H-indazol-1-yl)-N-methylbenzamide
N-[(3R)-1-cyclopentylpyrrolidin-3-yl]-4-(1H-indazol-1-yl)-N-methylbenzamide
4-(2H-indazol-2-yl)-N-(1-isopropylpiperidin-4-yl)-N-methylbenzamide
N-(1-cyclobutylpiperidin-4-yl)-4-(2H-indazol-2-yl)-N-methylbenzamide
N-(1-cyclopentylpiperidin-4-yl)-4-(2H-indazol-2-yl)-N-methylbenzamide
4-(1H-indazol-1-yl)-N-(1-isopropylpiperidin-4-yl)-N-methylbenzamide
N-(1-cyclobutylpiperidin-4-yl)-4-(1H-indazol-1-yl)-N-methylbenzamide
N-(1-cyclopentylpiperidin-4-yl)-4-(1H-indazol-1-yl)-N-methylbenzamide
4-(1H-indazol-1-ylmethyl)-N-[(3R)-1-isopropylpyrrolidin-3-yl]-N-methylbenzamide
N-[(3R)-1-cyclobutylpyrrolidin-3-yl]-4-(1H-indazol-1-ylmethyl)-N-methylbenzamide
N-[(3R)-1-cyclopentylpyrrolidin-3-yl]-4-(1H-indazol-1-ylmethyl)-N-methylbenzamide
4-(1H-indazol-1-ylmethyl)-N-(1-isopropylpiperidin-4-yl)-N-methylbenzamide
N-(1-cyclobutylpiperidin-4-yl)-4-(1H-indazol-1-ylmethyl)-N-methylbenzamide
N-(1-cyclopentylpiperidin-4-yl)-4-(1H-indazol-1-ylmethyl)-N-methylbenzamide
N-[(3R)-1-isopropylpyrrolidin-3-yl]-N-methyl-4-(1H-pyrazol-1-yl)benzamide
N-[(3R)-1-cyclobutylpyrrolidin-3-yl]-N-methyl-4-(1H-pyrazol-1-yl)benzamide
N-[(3R)-1-cyclopentylpyrrolidin-3-yl]-N-methyl-4-(1H-pyrazol-1-yl)benzamide
N-(1-isopropylpiperidin-4-yl)-N-methyl-4-(1H-pyrazol-1-yl)benzamide
N-(1-cyclobutylpiperidin-4-yl)-N-methyl-4-(1H-pyrazol-1-yl)benzamide
N-(1-cyclopentylpiperidin-4-yl)-N-methyl-4-(1H-pyrazol-1-yl)benzamide
N-methyl-N-[(3R)-1-(1-methylethyl)pyrrolidin-3-yl]-4-(1H-pyrazol-1-ylmethyl)benzamide
N-[(3R)-1-cyclobutylpyrrolidin-3-yl]-N-methyl-4-(1H-pyrazol-1-ylmethyl)benzamide
N-[(3R)-1-cyclopentylpyrrolidin-3-yl]-N-methyl-4-(1H-pyrazol-1-ylmethyl)benzamide
N-methyl-N-[1-(1-methylethyl)piperidin-4-yl]-4-(1H-pyrazol-1-ylmethyl)benzamide
N-(1-cyclobutylpiperidin-4-yl)-N-methyl-4-(1H-pyrazol-1-ylmethyl)benzamide
N-(1-cyclopentylpiperidin-4-yl)-N-methyl-4-(1H-pyrazol-1-ylmethyl)benzamide
3-fluoro-N-methyl-4-(2-methyl-1H-benzimidazol-1-yl)-N-[(3R)-1-(1-methylethyl)pyrrolidin-3-yl]benzamide
N-[(3R)-1-cyclobutylpyrrolidin-3-yl]-3-fluoro-N-methyl-4-(2-methyl-1H-benzimidazol-1-yl)benzamide
N,3-dimethyl-4-(2-methyl-1H-benzimidazol-1-yl)-N-[(3R)-1-(1-methylethyl)pyrrolidin-3-yl]benzamide
N-[(3R)-1-cyclobutylpyrrolidin-3-yl]-N,3-dimethyl-4-(2-methyl-1H-benzimidazol-1-yl)benzamide
3-methoxy-N-methyl-4-(2-methyl-1H-benzimidazol-1-yl)-N-[(3R)-1-(1-methylethyl)pyrrolidin-3-yl]benzamide
N-[(3R)-1-cyclobutylpyrrolidin-3-yl]-3-methoxy-N-methyl-4-(2-methyl-1H-benzimidazol-1-yl)benzamide
3-fluoro-N-methyl-4-(2-methyl-1H-benzimidazol-1-yl)-N-[1-(1-methylethyl)piperidin-4-yl]benzamide
N-(1-cyclopentylpiperidin-4-yl)-3-fluoro-N-methyl-4-(2-methyl-1H-benzimidazol-1-yl)benzamide
2-chloro-N-methyl-4-(2-methyl-1H-benzimidazol-1-yl)-N-[1-(1-methylethyl)piperidin-4-yl]benzamide
2-chloro-N-(1-cyclopentylpiperidin-4-yl)-N-methyl-4-(2-methyl-1H-benzimidazol-1-yl)benzamide
N,3-dimethyl-4-(2-methyl-1H-benzimidazol-1-yl)-N-[1-(1-methylethyl)piperidin-4-yl]benzamide
N-(1-cyclopentylpiperidin-4-yl)-N,3-dimethyl-4-(2-methyl-1H-benzimidazol-1-yl)benzamide
3-methoxy-N-methyl-4-(2-methyl-1H-benzimidazol-1-yl)-N-[1-(1-methylethyl)piperidin-4-yl]benzamide
N-(1-cyclopentylpiperidin-4-yl)-3-methoxy-N-methyl-4-(2-methyl-1H-benzimidazol-1-yl)benzamide
N-[(3R)-1-isopropylpyrrolidin-3-yl]-4-(2-methyl-1H-benzimidazol-1-yl)benzamide
N-[(3R)-1-cyclopentylpyrrolidin-3-yl]-4-(2-methyl-1H-benzimidazol-1-yl)benzamide
N-[(3R)-1-cyclohexylpyrrolidin-3-yl]-4-(2-methyl-1H-benzimidazol-1-yl)benzamide
N-ethyl-N-[(3R)-1-isopropylpyrrolidin-3-yl]-4-(2-methyl-1H-benzimidazol-1-yl)benzamide
N-[(3R)-1-cyclobutylpyrrolidin-3-yl]-N-ethyl-4-(2-methyl-1H-benzimidazol-1-yl)benzamide
N-[(3R)-1-cyclopentylpyrrolidin-3-yl]-N-ethyl-4-(2-methyl-1H-benzimidazol-1-yl)benzamide
N-[(3R)-1-cyclohexylpyrrolidin-3-yl]-N-ethyl-4-(2-methyl-1H-benzimidazol-1-yl)benzamide
2-chloro-N-methyl-4-(2-methyl-1H-benzimidazol-1-yl)-N-[(3R)-1-(1-methylethyl)pyrrolidin-3-yl]benzamide
2-chloro-N-[(3R)-1-cyclobutylpyrrolidin-3-yl]-N-methyl-4-(2-methyl-1H-benzimidazol-1-yl)benzamide;
(R)—N-(1-isopropylpyrrolidin-3-yl)-N-methyl-4-((2-methyl-1H-benzo[d]imidazol-1-yl)methyl)-1-naphthamide
(R)—N-(1-cyclobutylpyrrolidin-3-yl)-N-methyl-4-((2-methyl-1H-benzo[d]imidazol-1-yl)methyl)-1-naphthamide; and
N-(1-cyclopentylpiperidin-4-yl)-N-methyl-4-((2-methyl-1H-benzo[d]imidazol-1-yl)methyl)-1-naphthamide.

Additional exemplary embodiments of the present invention include a compound selected from the group consisting essentially of:
(R)—N-(1-cyclobutylpyrrolidin-3-yl)-2-methoxy-N-methyl-4-(2-methyl-1H-benzo[d]imidazol-1-yl)benzamide
(R)—N-(1-isopropylpyrrolidin-3-yl)-2-methoxy-N-methyl-4-(2-methyl-1H-benzo[d]imidazol-1-yl)benzamide
N—((R)-1-cyclobutylpyrrolidin-3-yl)-N-methyl-4-(2-methyl-1H-benzo[d]imidazol-1-yl)-3-(trifluoromethyl)benzamide
N—((R)-1-isopropylpyrrolidin-3-yl)-N-methyl-4-(2-methyl-1H-benzo[d]imidazol-1-yl)-3-(trifluoromethyl)benzamide
(R)—N-(1-cyclobutylpyrrolidin-3-yl)-N-methyl-4-(2-methyl-1H-benzo[d]imidazol-1-yl)-2-(trifluoromethyl)benzamide
(R)—N-(1-isopropylpyrrolidin-3-yl)-N-methyl-4-(2-methyl-1H-benzo[d]imidazol-1-yl)-2-(trifluoromethyl)benzamide
(R)—N-(1-cyclobutylpyrrolidin-3-yl)-N,2-dimethyl-4-(2-methyl-1H-benzo[d]imidazol-1-yl)benzamide
(R)—N-(1-isopropylpyrrolidin-3-yl)-N,2-dimethyl-4-(2-methyl-1H-benzo[d]imidazol-1-yl)benzamide
N-(1-cyclopentylpiperidin-4-yl)-2-methoxy-N-methyl-4-(2-methyl-1H-benzo[d]imidazol-1-yl)benzamide
N-(1-isopropylpiperidin-4-yl)-2-methoxy-N-methyl-4-(2-methyl-1H-benzo[d]imidazol-1-yl)benzamide
N-(1-cyclopentylpiperidin-4-yl)-N-methyl-4-(2-methyl-1H-benzo[d]imidazol-1-yl)-3-(trifluoromethyl)benzamide
N-(1-isopropylpiperidin-4-yl)-N-methyl-4-(2-methyl-1H-benzo[d]imidazol-1-yl)-3-(trifluoromethyl)benzamide
N-(1-cyclopentylpiperidin-4-yl)-N-methyl-4-(2-methyl-1H-benzo[d]imidazol-1-yl)-2-(trifluoromethyl)benzamide
N-(1-isopropylpiperidin-4-yl)-N-methyl-4-(2-methyl-1H-benzo[d]imidazol-1-yl)-2-(trifluoromethyl)benzamide
N-(1-cyclopentylpiperidin-4-yl)-N,2-dimethyl-4-(2-methyl-1H-benzo[d]imidazol-1-yl)benzamide
N-(1-isopropylpiperidin-4-yl)-N,2-dimethyl-4-(2-methyl-1H-benzo[d]imidazol-1-yl)benzamide
(R)—N-(1-cyclobutylpyrrolidin-3-yl)-N-methyl-4-((2-methyl-1H-benzo[d]imidazol-1-yl)methyl)-1-naphthamide
(R)—N-(1-isopropylpyrrolidin-3-yl)-N-methyl-4-((2-methyl-1H-benzo[d]imidazol-1-yl)methyl)-1-naphthamide
N-(1-isopropylpiperidin-4-yl)-N-methyl-4-((2-methyl-1H-benzo[d]imidazol-1-yl)methyl)-1-naphthamide
N-(1-cyclopentylpiperidin-4-yl)-N-methyl-4-((2-methyl-1H-benzo[d]imidazol-1-yl)methyl)-1-naphthamide
(R)—N-(1-cyclobutylpyrrolidin-3-yl)-N-methyl-3-(2-methyl-1H-benzo[d]imidazol-1-yl)benzamide
(R)—N-(1-isopropylpyrrolidin-3-yl)-N-methyl-3-(2-methyl-1H-benzo[d]imidazol-1-yl)benzamide
N-(1-cyclopentylpiperidin-4-yl)-N-methyl-3-(2-methyl-1H-benzo[d]imidazol-1-yl)benzamide
N-(1-isopropylpiperidin-4-yl)-N-methyl-3-(2-methyl-1H-benzo[d]imidazol-1-yl)benzamide
(R)—N-(1-cyclobutylpyrrolidin-3-yl)-4-((4-fluoro-1H-benzo[d]imidazol-1-yl)methyl)-N-methylbenzamide
(R)-4-((4-fluoro-1H-benzo[d]imidazol-1-yl)methyl)-N-(1-isopropylpyrrolidin-3-yl)-N-methylbenzamide
N-(1-cyclopentylpiperidin-4-yl)-4-((4-fluoro-1H-benzo[d]imidazol-1-yl)methyl)-N-methylbenzamide
4-((4-fluoro-1H-benzo[d]imidazol-1-yl)methyl)-N-(1-isopropylpiperidin-4-yl)-N-methylbenzamide
(R)—N-(1-cyclobutylpyrrolidin-3-yl)-4-((4-fluoro-2-methyl-1H-benzo[d]imidazol-1-yl)methyl)-N-methylbenzamide
(R)-4-((4-fluoro-2-methyl-1H-benzo[d]imidazol-1-yl)methyl)-N-(1-isopropylpyrrolidin-3-yl)-N-methylbenzamide
N-(1-cyclopentylpiperidin-4-yl)-4-((4-fluoro-2-methyl-1H-benzo[d]imidazol-1-yl)methyl)-N-methylbenzamide
4-((4-fluoro-2-methyl-1H-benzo[d]imidazol-1-yl)methyl)-N-(1-isopropylpiperidin-4-yl)-N-methylbenzamide
(R)—N-(1-cyclobutylpyrrolidin-3-yl)-4-((5-fluoro-1H-benzo[d]imidazol-1-yl)methyl)-N-methylbenzamide
(R)-4-((5-fluoro-1H-benzo[d]imidazol-1-yl)methyl)-N-(1-isopropylpyrrolidin-3-yl)-N-methylbenzamide
N-(1-cyclopentylpiperidin-4-yl)-4-((5-fluoro-1H-benzo[d]imidazol-1-yl)methyl)-N-methylbenzamide
4-((5-fluoro-1H-benzo[d]imidazol-1-yl)methyl)-N-(1-isopropylpiperidin-4-yl)-N-methylbenzamide
(R)—N-(1-cyclobutylpyrrolidin-3-yl)-4-((5-fluoro-2-methyl-1H-benzo[d]imidazol-1-yl)methyl)-N-methylbenzamide
(R)-4-((5-fluoro-2-methyl-1H-benzo[d]imidazol-1-yl)methyl)-N-(1-isopropylpyrrolidin-3-yl)-N-methylbenzamide
N-(1-cyclopentylpiperidin-4-yl)-4-((5-fluoro-2-methyl-1H-benzo[d]imidazol-1-yl)methyl)-N-methylbenzamide
4-((5-fluoro-2-methyl-1H-benzo[d]imidazol-1-yl)methyl)-N-(1-isopropylpiperidin-4-yl)-N-methylbenzamide
(R)—N-(1-cyclobutylpyrrolidin-3-yl)-4-((6-fluoro-1H-benzo[d]imidazol-1-yl)methyl)-N-methylbenzamide
(R)-4-((6-fluoro-1H-benzo[d]imidazol-1-yl)methyl)-N-(1-isopropylpyrrolidin-3-yl)-N-methylbenzamide
N-(1-cyclopentylpiperidin-4-yl)-4-((6-fluoro-1H-benzo[d]imidazol-1-yl)methyl)-N-methylbenzamide
N-(1-cyclopentylpiperidin-4-yl)-4-((6-fluoro-1H-benzo[d]imidazol-1-yl)methyl)-N-methylbenzamide
(R)—N-(1-cyclobutylpyrrolidin-3-yl)-4-((6-fluoro-2-methyl-1H-benzo[d]imidazol-1-yl)methyl)-N-methylbenzamide
(R)-4-((6-fluoro-2-methyl-1H-benzo[d]imidazol-1-yl)methyl)-N-(1-isopropylpyrrolidin-3-yl)-N-methylbenzamide
N-(1-cyclopentylpiperidin-4-yl)-4-((6-fluoro-2-methyl-1H-benzo[d]imidazol-1-yl)methyl)-N-methylbenzamide
4-((6-fluoro-2-methyl-1H-benzo[d]imidazol-1-yl)methyl)-N-(1-isopropylpiperidin-4-yl)-N-methylbenzamide
(R)—N-(1-cyclobutylpyrrolidin-3-yl)-4-((7-fluoro-1H-benzo[d]imidazol-1-yl)methyl)-N-methylbenzamide
(R)—N-(1-cyclobutylpyrrolidin-3-yl)-4-((7-fluoro-1H-benzo[d]imidazol-1-yl)methyl)-N-methylbenzamide
N-(1-cyclopentylpiperidin-4-yl)-4-((7-fluoro-1H-benzo[d]imidazol-1-yl)methyl)-N-methylbenzamide
4-((7-fluoro-1H-benzo[d]imidazol-1-yl)methyl)-N-(1-isopropylpiperidin-4-yl)-N-methylbenzamide
(R)—N-(1-cyclobutylpyrrolidin-3-yl)-4-((7-fluoro-2-methyl-1H-benzo[d]imidazol-1-yl)methyl)-N-methylbenzamide
(R)-4-((7-fluoro-2-methyl-1H-benzo[d]imidazol-1-yl)methyl)-N-(1-isopropylpyrrolidin-3-yl)-N-methylbenzamide
N-(1-cyclopentylpiperidin-4-yl)-4-((7-fluoro-2-methyl-1H-benzo[d]imidazol-1-yl)methyl)-N-methylbenzamide
4-((7-fluoro-2-methyl-1H-benzo[d]imidazol-1-yl)methyl)-N-(1-isopropylpiperidin-4-yl)-N-methylbenzamide
(R)-4-((1H-benzo[d]imidazol-1-yl)methyl)-N-(1-cyclobutylpyrrolidin-3-yl)-2-fluoro-N-methylbenzamide
(R)-4-((1H-benzo[d]imidazol-1-yl)methyl)-2-fluoro-N-(1-isopropylpyrrolidin-3-yl)-N-methylbenzamide
4-((1H-benzo[d]imidazol-1-yl)methyl)-N-(1-cyclopentylpiperidin-4-yl)-2-fluoro-N-methylbenzamide
4-((1H-benzo[d]imidazol-1-yl)methyl)-2-fluoro-N-(1-isopropylpiperidin-4-yl)-N-methylbenzamide
(R)—N-(1-cyclobutylpyrrolidin-3-yl)-2-fluoro-N-methyl-4-((2-methyl-1H-benzo[d]imidazol-1-yl)methyl)benzamide
(R)-2-fluoro-N-(1-isopropylpyrrolidin-3-yl)-N-methyl-4-((2-methyl-1H-benzo[d]imidazol-1-yl)methyl)benzamide
N-(1-cyclopentylpiperidin-4-yl)-2-fluoro-N-methyl-4-((2-methyl-1H-benzo[d]imidazol-1-yl)methyl)benzamide
2-fluoro-N-(1-isopropylpiperidin-4-yl)-N-methyl-4-((2-methyl-1H-benzo[d]imidazol-1-yl)methyl)benzamide
(R)-4-((1H-benzo[d]imidazol-1-yl)methyl)-N-(1-cyclobutylpyrrolidin-3-yl)-3-fluoro-N-methylbenzamide
(R)-4-((1H-benzo[d]imidazol-1-yl)methyl)-3-fluoro-N-(1-isopropylpyrrolidin-3-yl)-N-methylbenzamide
4-((1H-benzo[d]imidazol-1-yl)methyl)-N-(1-cyclopentylpiperidin-4-yl)-3-fluoro-N-methylbenzamide
4-((1H-benzo[d]imidazol-1-yl)methyl)-3-fluoro-N-(1-isopropylpiperidin-4-yl)-N-methylbenzamide
(R)—N-(1-cyclobutylpyrrolidin-3-yl)-3-fluoro-N-methyl-4-((2-methyl-1H-benzo[d]imidazol-1-yl)methyl)benzamide
(R)-3-fluoro-N-(1-isopropylpyrrolidin-3-yl)-N-methyl-4-((2-methyl-1H-benzo[d]imidazol-1-yl)methyl)benzamide
N-(1-cyclopentylpiperidin-4-yl)-3-fluoro-N-methyl-4-((2-methyl-1H-benzo[d]imidazol-1-yl)methyl)benzamide
3-fluoro-N-(1-isopropylpiperidin-4-yl)-N-methyl-4-((2-methyl-1H-benzo[d]imidazol-1-yl)methyl)benzamide
(R)-4-((1H-benzo[d]imidazol-1-yl)methyl)-N-(1-cyclobutylpyrrolidin-3-yl)-2-methoxy-N-methylbenzamide
(R)-4-((1H-benzo[d]imidazol-1-yl)methyl)-N-(1-isopropylpyrrolidin-3-yl)-2-methoxy-N-methylbenzamide
4-((1H-benzo[d]imidazol-1-yl)methyl)-N-(1-cyclopentylpiperidin-4-yl)-2-methoxy-N-methylbenzamide
4-((1H-benzo[d]imidazol-1-yl)methyl)-N-(1-isopropylpiperidin-4-yl)-2-methoxy-N-methylbenzamide
(R)—N-(1-cyclobutylpyrrolidin-3-yl)-2-methoxy-N-methyl-4-((2-methyl-1H-benzo[d]imidazol-1-yl)methyl)benzamide
(R)—N-(1-isopropylpyrrolidin-3-yl)-2-methoxy-N-methyl-4-((2-methyl-1H-benzo[d]imidazol-1-yl)methyl)benzamide
N-(1-cyclopentylpiperidin-4-yl)-2-methoxy-N-methyl-4-((2-methyl-1H-benzo[d]imidazol-1-yl)methyl)benzamide
N-(1-isopropylpiperidin-4-yl)-2-methoxy-N-methyl-4-((2-methyl-1H-benzo[d]imidazol-1-yl)methyl)benzamide
(R)-4-((1H-benzo[d]imidazol-1-yl)methyl)-3-chloro-N-(1-cyclobutylpyrrolidin-3-yl)-N-methylbenzamide
(R)-4-((1H-benzo[d]imidazol-1-yl)methyl)-3-chloro-N-(1-isopropylpyrrolidin-3-yl)-N-methylbenzamide
4-((1H-benzo[d]imidazol-1-yl)methyl)-3-chloro-N-(1-cyclopentylpiperidin-4-yl)-N-methylbenzamide
4-((1H-benzo[d]imidazol-1-yl)methyl)-3-chloro-N-(1-isopropylpiperidin-4-yl)-N-methylbenzamide
(R)-3-chloro-N-(1-cyclobutylpyrrolidin-3-yl)-N-methyl-4-((2-methyl-1H-benzo[d]imidazol-1-yl)methyl)benzamide
(R)-3-chloro-N-(1-isopropylpyrrolidin-3-yl)-N-methyl-4-((2-methyl-1H-benzo[d]imidazol-1-yl)methyl)benzamide
3-chloro-N-(1-cyclopentylpiperidin-4-yl)-N-methyl-4-((2-methyl-1H-benzo[d]imidazol-1-yl)methyl)benzamide
3-chloro-N-(1-isopropylpiperidin-4-yl)-N-methyl-4-((2-methyl-1H-benzo[d]imidazol-1-yl)methyl)benzamide
4-((1H-benzo[d]imidazol-1-yl)methyl)-N-(1-isopropylpiperidin-3-yl)-N-methylbenzamide
4-((1H-benzo[d]imidazol-1-yl)methyl)-N-(1-cyclobutylpiperidin-3-yl)-N-methylbenzamide
4-((1H-benzo[d]imidazol-1-yl)methyl)-N-(1-cyclopentylpiperidin-3-yl)-N-methylbenzamide
N-(1-isopropylpiperidin-3-yl)-N-methyl-4-((2-methyl-1H-benzo[d]imidazol-1-yl)methyl)benzamide
N-(1-cyclobutylpiperidin-3-yl)-N-methyl-4-((2-methyl-1H-benzo[d]imidazol-1-yl)methyl)benzamide
N-(1-cyclopentylpiperidin-3-yl)-N-methyl-4-((2-methyl-1H-benzo[d]imidazol-1-yl)methyl)benzamide
4-(1H-benzo[d]imidazol-1-yl)-N-(1-isopropylpiperidin-3-yl)-N-methylbenzamide
4-(1H-benzo[d]imidazol-1-yl)-N-(1-cyclobutylpiperidin-3-yl)-N-methylbenzamide
4-(1H-benzo[d]imidazol-1-yl)-N-(1-cyclopentylpiperidin-3-yl)-N-methylbenzamide
N-(1-isopropylpiperidin-3-yl)-N-methyl-4-(2-methyl-1H-benzo[d]imidazol-1-yl)benzamide
N-(1-cyclobutylpiperidin-3-yl)-N-methyl-4-(2-methyl-1H-benzo[d]imidazol-1-yl)benzamide
N-(1-cyclopentylpiperidin-3-yl)-N-methyl-4-(2-methyl-1H-benzo[d]imidazol-1-yl)benzamide
(R)—N-(1-cyclobutylpyrrolidin-3-yl)-4-((6-methoxy-1H-benzo[d]imidazol-1-yl)methyl)-N-methylbenzamide
(R)—N-(1-cyclobutylpyrrolidin-3-yl)-4-((5-methoxy-1H-benzo[d]imidazol-1-yl)methyl)-N-methylbenzamide
(R)—N-(1-cyclobutylpyrrolidin-3-yl)-N-methyl-4-((5-methyl-1H-benzo[d]imidazol-1-yl)methyl)benzamide
(R)—N-(1-cyclobutylpyrrolidin-3-yl)-N-methyl-4-((6-methyl-1H-benzo[d]imidazol-1-yl)methyl)benzamide
(R)—N-(1-isopropylpyrrolidin-3-yl)-N-methyl-4-((6-methyl-1H-benzo[d]imidazol-1-yl)methyl)benzamide
(R)—N-(1-isopropylpyrrolidin-3-yl)-N-methyl-4-((5-methyl-1H-benzo[d]imidazol-1-yl)methyl)benzamide
(R)—N-(1-isopropylpyrrolidin-3-yl)-4-((5-methoxy-1H-benzo[d]imidazol-1-yl)methyl)-N-methylbenzamide
(R)—N-(1-isopropylpyrrolidin-3-yl)-4-((6-methoxy-1H-benzo[d]imidazol-1-yl)methyl)-N-methylbenzamide
N-(1-isopropylpiperidin-4-yl)-N-methyl-4-((6-methyl-1H-benzo[d]imidazol-1-yl)methyl)benzamide
N-(1-isopropylpiperidin-4-yl)-4-((6-methoxy-1H-benzo[d]imidazol-1-yl)methyl)-N-methylbenzamide
N-(1-isopropylpiperidin-4-yl)-N-methyl-4-((5-methyl-1H-benzo[d]imidazol-1-yl)methyl)benzamide
N-(1-isopropylpiperidin-4-yl)-4-((5-methoxy-1H-benzo[d]imidazol-1-yl)methyl)-N-methylbenzamide
(R)—N-(1-cyclobutylpyrrolidin-3-yl)-4-((6-methoxy-2-methyl-1H-benzo[d]imidazol-1-yl)methyl)-N-methylbenzamide
(R)—N-(1-cyclobutylpyrrolidin-3-yl)-4-((2,6-dimethyl-1H-benzo[d]imidazol-1-yl)methyl)-N-methylbenzamide
(R)—N-(1-cyclobutylpyrrolidin-3-yl)-4-((2,5-dimethyl-1H-benzo[d]imidazol-1-yl)methyl)-N-methylbenzamide
(R)—N-(1-cyclobutylpyrrolidin-3-yl)-4-((5-methoxy-2-methyl-1H-benzo[d]imidazol-1-yl)methyl)-N-methylbenzamide
(R)—N-(1-isopropylpyrrolidin-3-yl)-4-((5-methoxy-2-methyl-1H-benzo[d]imidazol-1-yl)methyl)-N-methylbenzamide
(R)-4-((2,5-dimethyl-1H-benzo[d]imidazol-1-yl)methyl)-N-(1-isopropylpyrrolidin-3-yl)-N-methylbenzamide
(R)-4-((2,6-dimethyl-1H-benzo[d]imidazol-1-yl)methyl)-N-(1-isopropylpyrrolidin-3-yl)-N-methylbenzamide
(R)—N-(1-isopropylpyrrolidin-3-yl)-4-((6-methoxy-2-methyl-1H-benzo[d]imidazol-1-yl)methyl)-N-methylbenzamide
N-(1-isopropylpiperidin-4-yl)-4-((5-methoxy-2-methyl-1H-benzo[d]imidazol-1-yl)methyl)-N-methylbenzamide
4-((2,5-dimethyl-1H-benzo[d]imidazol-1-yl)methyl)-N-(1-isopropylpiperidin-4-yl)-N-methylbenzamide
4-((2,6-dimethyl-1H-benzo[d]imidazol-1-yl)methyl)-N-(1-isopropylpiperidin-4-yl)-N-methylbenzamide; and
N-(1-isopropylpiperidin-4-yl)-4-((6-methoxy-2-methyl-1H-benzo[d]imidazol-1-yl)methyl)-N-methylbenzamide; or
   a stereoisomer thereof or a pharmaceutically acceptable salt thereof.

Another aspect of the invention provides a method for the treatment of a cognitive disorder related to or affected by the Histamine-3 (H₃) receptor in a patient in need thereof which comprises providing to said patient a therapeutically effective amount of a compound of formula I or any other embodiment thereof described herein. In a more particular embodiment, said disorder is a neurodegenerative disorder. More particular still, said disorder is mild cognitive impairment (MCI), dementia, delirium, amnestic disorder, Alzheimer's disease (AD), Parkinson's disease (PD), Huntington's disease (HD), memory disorder, memory deficits associated with depression, schizophrenia, a psychotic disorder, paranoia, mano-depressive illness, attention deficit hyperactivity disorder (ADHD), dyslexia, developmental disorders, Down's syndrome, Fragile X syndrome, loss of executive function, loss of learned information, vascular dementia, cognitive decline, neurodegenerative disorder, HIV-induced dimentia, head trauma, Pick's disease, Creutzfeldt-Jakob disease, Body dementia, vascular dementia, surgical procedure-induced cognitive dysfunction, traumatic brain injury or stroke. In another more particular embodiment, said disorder is selected from the group consisting of: Alzheimer's disease, attention deficit disorder, schizophrenia; Parkinsons' disease, frontal temporal dementia or depression.

Another aspect of the invention provides a method for the inhibition of an H₃ receptor comprising contacting said receptor with an effective amount of a compound of formula I or any other embodiment thereof described herein.

An additional aspect of the invention provides a pharmaceutical composition which comprises a pharmaceutically acceptable carrier and an effective amount of a compound of formula I or any other embodiment thereof described herein.

“Treating” or “treatment” of a disease in a subject refers to 1) preventing the disease from occurring in a subject that is predisposed or does not yet display symptoms of the disease; 2) inhibiting the disease or arresting its development; or 3) ameliorating or causing regression of the disease.

A “cognitive disease,” “cognitive dysfunction,” or “cognition-related disorder” is a disease or disorder affecting mental processes such as memory, attention, perception, action, problem solving and mental imagery. Cognitive dysfunction generally originates in the central nervous system and can be influenced or derived from neurodegeneration. Particular cognition-related disorders (e.g., cognitive dysfunction) include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorders (e.g., attention deficit hyperactivity disorder (ADHD)) and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the embodiments described herein.

An optionally substituted moiety may be substituted with one or more substituents. The substituent groups, which are optionally present, may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl or cycloalkyl groups, preferably halogen atoms or lower alkyl or lower alkoxy groups. Unless otherwise specified, typically, 0-4 substituents may be present. When any of the foregoing substituents represents or contains an alkyl substituent group, this may be linear or branched and may contain up to 12 carbon atoms, preferably up to 6 carbon atoms, more preferably up to 4 carbon atoms.

Preferably, optionally substituted refers to the replacement of 0-4 hydrogen atoms with 0-4 groups selected from C₁-C₆ alkyl, C₃-C₆ cycloalkyl, C₂-C₆ alkenyl, C₂-C₆ alkynyl, halo, nitro, cyano, hydroxy, C₆-C₁₀ aryl, a 3-10 membered heterocyclyl ring, a 5-10 membered heteroaryl ring, —N(R^{a})ₘ, —C(O)R^{b}, —OR^{c} and —S(O)ₚR^{d}; wherein each R^{a} is independently H, C₁-C₄ alkyl, —CHO, —C(O)(C₁-C₄ alkyl), or —CO₂(C₁-C₄ alkyl); each R^{b} is independently H, —OH, —O(C₁-C₄), C₁-C₄ alkyl, —NH₂, —NH(C₁-C₄ alkyl), or —N(C₁-C₄ alkyl)₂; each R^{c} is independently H, C₁-C₄ alkyl, C₁-C₄ haloalkyl, —CHO or —C(O)(C₁-C₄ alkyl); each R^{d} is independently H, C₁-C₄ alkyl, or —OH; and p is 0, 1 or 2.

As used herein, the term “alkyl” includes both a (C₁-C₁₀) straight chain and a (C₃-C₁₂) branched chain saturated hydrocarbon moiety. Preferred alkyl groups have one to six carbon atoms (C₁-C₆ alkyl). Examples of saturated hydrocarbon alkyl moieties include, but are not limited to, methyl, ethyl, n-propyl, isopropyl, n-butyl, tert-butyl, isobutyl, sec-butyl; n-pentyl, n-hexyl, or the like.

“Alkoxy” refers to the group —O-alkyl wherein alkyl is defined herein. Preferred alkoxy groups have 1 to 6 carbon atoms (C₁-C₆ alkoxy). Alkoxy includes, by way of example, methoxy, ethoxy, n-propoxy, isopropoxy, n-butoxy, t-butoxy, sec-butoxy, and n-pentoxy.

“Amino” refers to the group —NH₂.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups are C₆-C₁₀ aryl groups and include phenyl and naphthyl.

“Arylalkyl” refers to an aryl group as defined herein appended at any suitable position to an alkyl group, wherein the point of attachment to the base-compound is at the alkyl group. Preferred arylalkyl groups have 7 to 14 carbon atoms (C₇-C₁₄ arylalkyl), more preferably the aryl portion is phenyl (C₆) and the alkyl portion is C₁-C₂. In such embodiments the group is C₇-C₉ arylalkyl. Examples of arylalkyl groups include benzyl and phenethyl.

“Alkenyl” refers to alkenyl groups having from 2 to 6 carbon atoms (C₂-C₆ alkenyl) and preferably 2 to 4 carbon atoms (C₂-C₄ alkenyl) and having at least 1 and preferably from 1 to 2 sites of alkenyl unsaturation. Such groups are exemplified, for example, by vinyl, allyl, and but-3-en-1-yl.

“Alkynyl” refers to alkynyl groups having from 2 to 6 carbon atoms (C₂-C₆ alkynyl) and preferably 2 to 3 carbon atoms (C₂-C₃ alkynyl) and having at least 1 and preferably from 1 to 2 sites of alkynyl unsaturation.

“Acyl” refers to the groups H—C(O)—, alkyl-C(O)—, alkenyl-C(O)—, alkynyl-C(O)—, cycloalkyl-C(O)—, cycloalkenyl-C(O)—, aryl-C(O)—, 5-7 membered heteroaryl-C(O)—, 5-7 membered heterocyclic-C(O)—, wherein alkyl, alkenyl, alkynyl, cycloalkyl, cycloalkenyl, aryl, heteroaryl, and heterocyclic are as defined herein. Acyl includes the “acetyl” group CH₃C(O)—.

“Cyano” or “nitrile” refers to the group —CN.

“Cycloalkenyl” refers to cyclic alkyl groups of from 3 to 10 carbon atoms having single or multiple cyclic rings including fused, bridged, and spiro ring systems which contain at least one double bond. Preferred cycloalkenyl groups have 3 to 6 carbon atoms (C₃-C₆ cycloalkenyl) and contain one double bond. Examples of suitable cycloalkenyl groups include, for instance, cyclobutenyl, cyclopentenyl, cyclohexenyl, and cyclooctenyl.

“Hydroxy” or “hydroxyl” refers to the group —OH.

“Nitro” refers to the group —NO₂.

“Oxo” refers to the atom (═O) or (—O⁻). As an activating group, ‘oxo’ groups are amenable to reductive amination by nucleophilic amine groups to form alkylamino or aminoalkyl substituents. Preferably, the reductive amination step takes place in the presence of a boron-containing reducing agent.

“Spirocyclyl” refers to divalent saturated cyclic group from 3 to 10 carbon atoms having a cycloalkyl or heterocyclyl ring with a spiro union (the union formed by a single atom which is the only common member of the rings) as exemplified by the following structure:

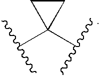

As used herein, the term “haloalkyl” designates a CₙH₂ₙ₊₁ group having from one to 2n+1 halogen atoms which may be the same or different. Preferably, haloalkyl groups have one to six carbon atoms (C₁-C₆ haloalkyl). Examples of haloalkyl groups include CF₃, CH₂Cl, C₂H₃BrCl, C₃H₅F₂, or the like.

The term “halogen” or “halo”, as used herein, designates fluorine, chlorine, bromine, and iodine.

The term “cycloalkyl”, as used herein, refers to a monocyclic, bicyclic, tricyclic, fused, bridged, or spiro monovalent saturated hydrocarbon moiety of 3-10 carbon atoms (C₃-C₁₀ cycloalkyl). Examples of cycloalkyl moieties include, but are not limited to cyclopropyl, cyclobutyl, cyclopentyl, cyclohexyl, cycloheptyl, norbornyl, adamantyl, spiro[4.5]decanyl, or the like.

The term “cycloheteroalkyl,” “heterocyclyl,” “heterocycloalkyl,” “heterocyclo” or “heterocyclylalkyl” as used herein, designates a C₃-C₁₀ cycloalkyl ring system containing 1, 2, 3 or 4 heteroatoms, which may be the same or different, selected from N, O or S and optionally containing one double bond. Where the cycloheteroalkyl groups are polycyclic (e.g. bicyclic), one of the rings may be aromatic so long as the ring which is the point of attachment for the cycloheteroalkyl group is not aromatic (e.g. 1,2,3,4-tetrahydroquinolin-3-yl). Exemplary of the cycloheteroalkyl ring systems included in the term as designated herein are the following rings wherein X₁ is NR, O or S and R is H or an optional substituent as defined hereinabove.

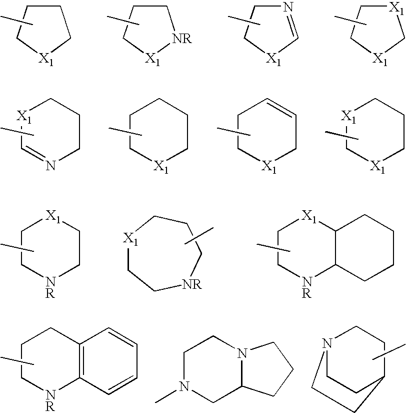

The term “heteroaryl” as used herein designates an aromatic heterocyclic ring system, which may be a single ring (monocyclic) or multiple rings (bicyclic, up to three rings) fused together or linked covalently. Preferably, heteroaryl is a 5- to 6-membered monocyclic ring or a 9- to 10-membered bicyclic ring system. Where the heteroaryl groups are polycyclic (e.g. bicyclic), one of the rings may be aromatic so long as the ring which is the point of attachment for the heteroaryl group is aromatic (e.g. 1,2,3,4-tetrahydro-1,8-naphthyridin-6-yl). The rings may contain from one to four hetero atoms selected from nitrogen, oxygen, or sulfur, wherein the nitrogen or sulfur atoms are optionally oxidized, or the nitrogen atom is optionally quarternized. Examples of heteroaryl moieties include, but are not limited to, heterocycles such as furan, thiophene, pyrrole, pyrazole, imidazole, oxazole, isoxazole, thiazole, isothiazole, oxadiazole, triazole, pyridine, pyrimidine, pyrazine, pyridazine, benzimidazole, benzoxazole, benzisoxazole, benzothiazole, benzofuran, benzothiophene, thianthrene, dibenzofuran, dibenzothiophene, indole, indazole, quinoline, isoquinoline, quinazoline, quinoxaline, purine, or the like.

Exemplary of the monocyclic 5-membered aromatic ring system formed when R₃ and R₄ are taken together with the nitrogen atom to which they are attached are pyrrole, pyrazole, imidazole, oxazole, isoxazole, thiazole, isothiazole, oxadiazole, triazole or the like. Exemplary of the fused bicyclic or tricyclic 9- to 15-membered aromatic ring system formed when R₃ and R₄ are taken together with the nitrogen atom to which they are attached are indolyl, indazolyl, benzimidazolyl, tetrahydrocarbazolyl, hexahydroindolizinoindolonyl, tetrahydropyranoindolyl, azaindolyl, imidazopyridinyl, indolinyl, tetrahydroquinolinlyl, pyridoindolyl, dihydrodibenzoazepinyl, or the like.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Patient” or “subject” refers to mammals and includes humans and non-human mammals, such as dogs, cats, mice, rats, cows, rabbits and monkeys.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of another example, the term “5-7 membered heteroaryl or heterocyclyl group” is specifically intended to individually disclose a heteroaryl or heterocyclyl group having 5, 6, 7, 5-7, and 5-6 ring atoms.

“Stereoisomer” or “stereoisomers” refer to compounds that differ in the chirality or atomic connectivity at one or more stereocenters. Stereoisomers include enantiomers, diastereomers as well as cis-trans (E/Z) isomerism. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C— or ¹⁴C-enriched carbon are within the scope of this invention.

The compounds of the present invention may be converted to salts, in particular pharmaceutically acceptable salts using art recognized procedures. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds or their pharmaceutically acceptable salts, are also included. The term “pharmaceutically acceptable salt”, as used herein, refers to salts derived from organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains a carboxylate or phenolic moiety, or similar moiety capable of forming base addition salts.

Compounds of the invention include esters, carbamates or other conventional prodrug forms, which in general, are functional derivatives of the compounds of the invention and which are readily converted to the inventive active moiety in vivo. Correspondingly, the method of the invention embraces the treatment of the various conditions described hereinabove with a compound of formula I or with a compound which is not specifically disclosed but which, upon administration, converts to a compound of formula I in vivo. Also included are metabolites of the compounds of the present invention defined as active species produced upon introduction of these compounds into a biological system.

Advantageously, the present invention provides a process to prepare compounds of formula I, which, in one embodiment comprises reacting a benzoic acid of formula II with an azacyclylamine of formula III in the presence of a coupling agent optionally in the presence of a solvent.

In one embodiment, the invention provides a process for the preparation of a compound of formula I

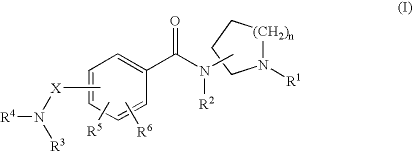

wherein
X is (CR⁷R⁸)ₘ, CO or SO₂;
m is 0 or 1;
n is 1, 2 or 3;
R¹ is H, C₁-C₆ alkyl, C₁-C₆ haloalkyl, C₃-C₁₀ cycloalkyl or a 3-10 membered cycloheteroalkyl each group optionally substituted;
R² is H or C₁-C₆ alkyl or C₃-C₁₀ cycloalkyl each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen, C₁-C₆ alkyl, C₁-C₆ alkoxy or C₃-C₁₀ cycloalkyl each optionally substituted; or R⁵ and R⁶ are taken together with the atoms to which they are attached to form an optionally substituted phenyl ring;
R⁷ and R⁸ are each independently H, halogen or C₁-C₆ alkyl or C₃-C₁₀ cycloalkyl each group optionally substituted; or

a stereoisomer, tautomer or pharmaceutically acceptable salt thereof;

which process comprises reacting a compound of formula

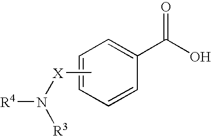

wherein X, R³ and R⁴ are as described hereinabove for formula I with an azacyclylamine of formula

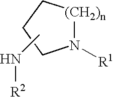

wherein n, R¹ and R² are as described hereinabove for formula I in the presence of a coupling agent optionally in the presence of a solvent.

In another embodiment, the present invention provides a process for the preparation of a compound of formula I, said process comprising reacting a compound of formula II

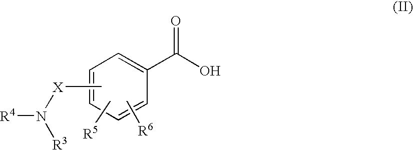

wherein X, R³, R⁴, R⁵ and R⁶ are as described hereinabove for formula I with an azacyclylamine of formula III

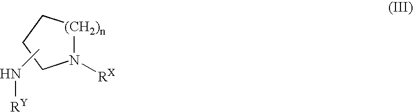

in the presence of a coupling agent and optionally in the presence of a solvent to form a compound of formula IIIa:

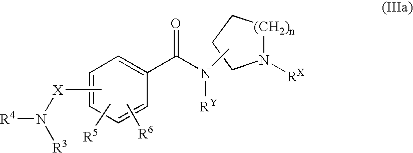

wherein,

R^{X} is R¹ or a protecting group;

R^{Y} is H or C₁-C₆ alkyl or C₃-C₁₀ cycloalkyl each group optionally substituted;

wherein, if R^{Y} is H and R² in the compound of formula I is other than H, than the process further comprises:

reacting activated-R² with the compound of formula IIIa, to form a compound of formula IIIb:

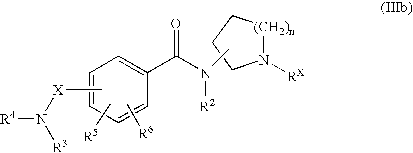

wherein if R^{X} is R¹, then the compound of formula I is formed; or

if R^{X} is a protecting group, then the process further comprises:

deprotecting the compound of formula IIIb to form a deprotected compound; and

if R¹ in the compound of formula I is H, then the compound of formula I is formed; or

if R¹ in the compound of formula I is other than H, then the process further comprises reacting the deprotected compound with activated-R¹;

wherein the compound of formula I is formed.

In a more particular embodiment of the above-process:
(a) R^{X} is a protecting group and the protecting group is t-butoxycarbonyl (Boc), benzyl, acetyl, p-methoxybenzyl (PMB), C₁-C₆ alkyl, 9-fluoroenylmethoxycarbonyl (Fmoc), benzyloxycarbonyl (Cbz), trifluoroacetyl, tosyl or trityl;
(b) R^{Y} is H;
(c) activated-R² is halo-R², tosylate-R², R²-anhydride, mesylate-R², or triflate-R²;
(d) activated-R¹ is halo-R¹ or oxo-R¹;
(e) the deprotecting step comprises contacting the compound of formula IIIb with an acid;
(f) activated-R¹ is oxo-R¹ and the reacting the deprotected compound with activated-R¹ step comprises a reductive amination reaction in the presence of a boron-reducing agent;
(g) any of the process steps are performed in a protic solvent, an aprotic solvent, a polar solvent, a nonpolar solvent, a protic polar solvent, an aprotic nonpolar solvent, or an aprotic polar solvent;
(h) any of the process steps includes a purification step comprising at least one of: filtration, extraction, chromatography, trituration, or recrystalization; and/or
(i) any of the process steps includes an analytical step comprising liquid chromatography (LC), mass spectroscopy (MS), liquid chromatography/mass spectroscopy (LC/MS), gas chromatography (GC), gas chromatography/mass spectroscopy (GC/MS), nuclear magnetic resonance (NMR), thin layer chromatography (TLC), melting point (MP) analysis, optical rotation (OR) or elemental analysis.

A reaction scheme for the preparation of a compound of formula I is shown in scheme I.

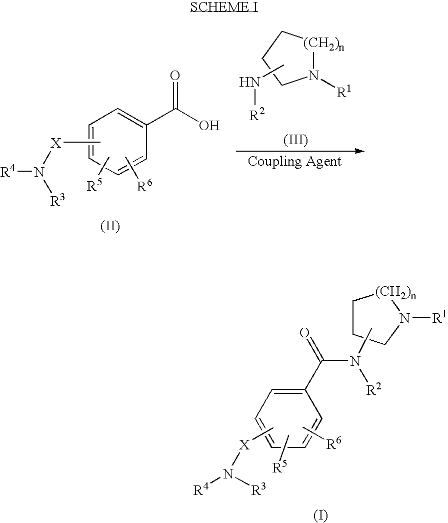

Coupling agents suitable for use in the method of invention include 2-(1H-benzotriazol-1-yl)-1,1,3,3-tetramethyluronium tetrafluoroborate, benzotriazol-1-yl-oxytripyrrolidinophosphonium hexafluorophosphate or the like, preferably 2-(1H-benzotriazol-1-yl)-1,1,3,3-tetramethyluronium tetrafluoroborate. Solvents suitable for use in the method of the invention include N,N-dimethylformamide, tetrahydrofuran, or the like.

Compounds of formula II wherein X is (CR⁷R⁸)ₘ (IIa) may be readily prepared by reacting a compound, HNR³R⁴, with a benzoate of formula IV in the presence of a base such as K₂CO₃ to give the corresponding substituted benzoate and hydrolyzing said substituted benzoate with a suitable base such as NaOH or LiOH to give the desired compound of formula IIa. The reaction is shown in scheme II wherein R is C₁-C₄ alkyl and Hal is Cl, Br or I.

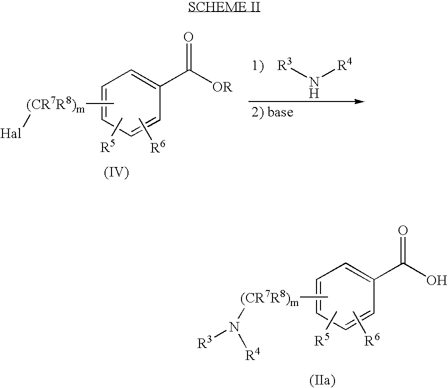

Alternatively, compounds of formula I may be prepared by reacting a benzoic acid of formula II with a protected azacyclylamine of formula V in the presence of a coupling agent, as described in scheme I, to give the protected aminoamide of formula VI, reacting said formula VI amide with an alkylating agent, R²-Hal, wherein Hal is Br or I to give the compound of formula VII; deprotecting said formula VII compound to give the corresponding free amine and reacting said amine with an aldehyde of formula VIII or a ketone of formula IX in the presence of a borohydride salt such as NaBH₃CN or NaBH(OAc)₃ to give the desired compound of formula I. The reaction is shown in scheme III wherein P represents a protecting group; Hal represents Br or I; and R^{a} represents R¹ minus one carbon atom (R¹—C₁).

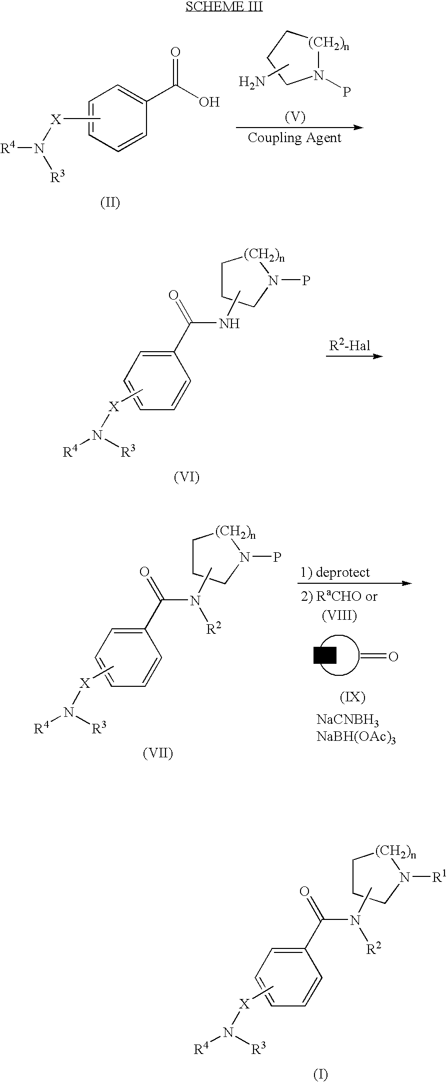

Protecting groups useful in the reactions described hereinabove include t-butoxycarbonyl (Boc), benzyl, acetyl, benzyloxycarbonyl, or any conventional group known to protect a basic nitrogen in standard synthetic procedures, preferably t-butoxycarbonyl.

Compounds of formula I wherein X is CO (Ib) may be prepared by reacting a halobenzoic acid of formula X with an azacyclylamine of formula III in the presence of a coupling agent, as described hereinabove in schemes I and II, to give the corresponding amide of formula XI; reacting the formula XI amide with carbon monoxide and methanol in the presence of a palladium catalyst to give the benzoate of formula XII; hydrolyzing the formula XII benzoate with base to give the corresponding benzoic acid; reacting said benzoic acid with thionyl chloride to give the benzoic acid chloride of formula XIII; reacting the formula XIII acid chloride with a compound, HNR³R⁴, to give the desired compound of formula Ib. The reaction is shown in scheme IV wherein Hal represents Br or I.

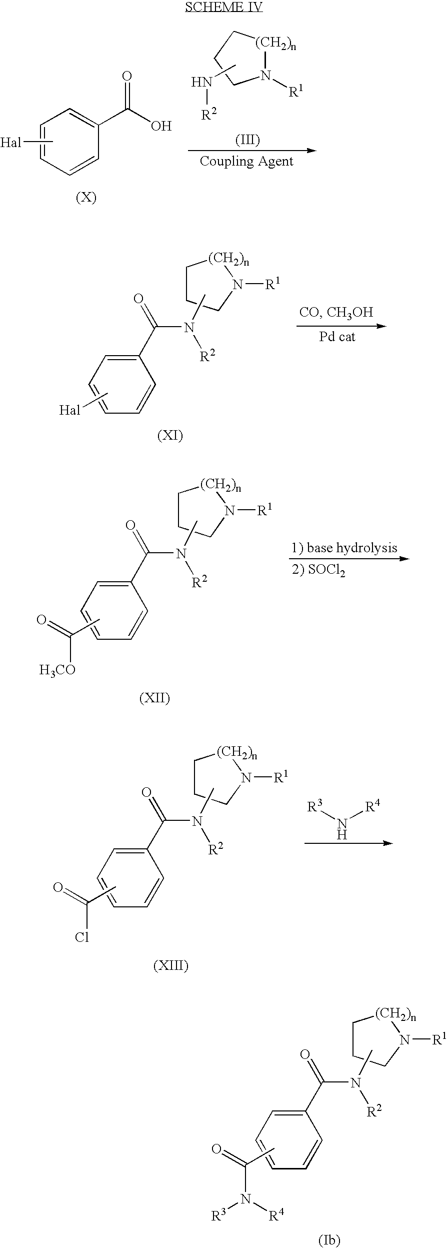

Compounds of formula I wherein X is SO₂ (Ic) may be prepared by reacting a phenylsulfonyl chloride of formula XIV with a compound, HNR₃R₄, to give the compound of formula XV; hydrolysing the compound of formula XV to give the benzoic acid of formula XVI; reacting said formula XVI benzoic acid with a protected azacyclylamine of formula V in the presence of a coupling agent as described hereinabove in scheme III to give the compound of formula XVII; and converting said formula XVII compound to the desired compound of formula Ic via sequential alkylation, deprotection and reductive amination in the manner described hereinabove in scheme III. The reaction is shown in scheme V wherein R is C₁-C₄ alkyl, P is a protecting group, Hal is Br or I and R^{a} represents R¹ minus one carbon atom (R¹—C₁).

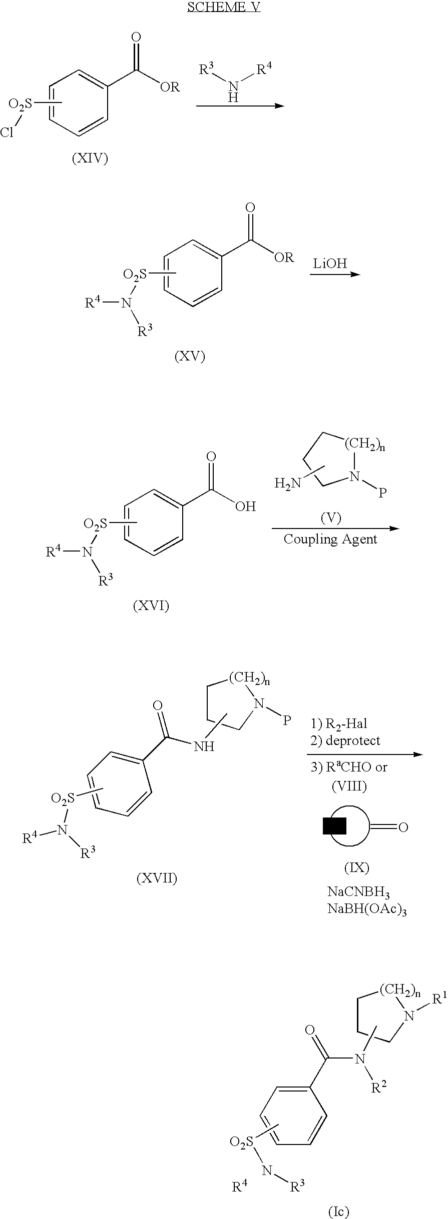

Advantageously, the formula I compounds of the invention are useful for the treatment of CNS disorders related to or affected by the Histamine-3 receptor including cognitive disorders, for example Alzheimer's disease, mild cognitive impairment, attention deficit hyperactivity disorder, schizophrenia, memory loss, sleep disorders, obesity, psychosis, dementia, depression, Parkinson's disease or the like. Accordingly, the present invention provides a method for the treatment of a disorder of the central nervous system related to or affected by the Histamine-3 receptor in a patient in need thereof which comprises providing said patient a therapeutically effective amount of a compound of formula I as described hereinabove. The compounds may be provided by oral or parenteral administration or in any common manner known to be an effective administration of a therapeutic agent to a patient in need thereof.

The term “providing” as used herein with respect to providing a compound or substance embraced by the invention, designates either directly administering such a compound or substance, or administering a prodrug, derivative or analog which forms an equivalent amount of the compound or substance within the body.

The inventive method includes: a method for the treatment of schizophrenia; a method for the treatment of a disease associated with a deficit in memory, cognition or learning or a cognitive disorder such as Alzheimer's disease or attention deficit hyperactivity disorder; a method for the treatment of a mild cognitive disorder, a method for the treatment of a developmental disorder such as schizophrenia; a method for the treatment of psychosis; a method for the treatment of Parkinson's disease; a method for the treatment of depression; a method for the treatment of a sleep disorder or any other CNS disease or disorder associated with or related to the H₃ receptor.

In one embodiment, the present invention provides a method for treating attention deficit hyperactivity disorders (ADHD, also known as Attention Deficit Disorder or ADD) in both children and adults. Accordingly, in this embodiment, the present invention provides a method for treating attention deficit disorders in a pediatric patient.

The present invention therefore provides a method for the treatment of each of the conditions listed above in a patient, preferably in a human, said method comprises providing said patient a therapeutically effective amount of a compound of formula I as described hereinabove. The compounds may be provided by oral or parenteral administration or in any common manner known to be an effective administration of a therapeutic agent to a patient in need thereof.

The therapeutically effective amount provided in the treatment of a specific CNS disorder may vary according to the specific condition(s) being treated, the size, age and response pattern of the patient, the severity of the disorder, the judgment of the attending physician and the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

In actual practice, the compounds of the invention are provided by administering the compound or a precursor thereof in a solid or liquid form, either neat or in combination with one or more conventional pharmaceutical carriers or excipients. Accordingly, the present invention provides a pharmaceutical composition which comprises a pharmaceutically acceptable carrier and an effective amount of a compound of formula I as described hereinabove.

In one embodiment, the invention relates to compositions comprising at least one compound of formula I, or a pharmaceutically acceptable salt thereof, and one or more pharmaceutically acceptable carriers, excipients, or diluents. Such compositions include pharmaceutical compositions for treating or controlling disease states or conditions of the central nervous system. In certain embodiments, the compositions comprise mixtures of one or more compounds of formula I.

In certain embodiments, the invention relates to compositions comprising at least one compound of formula I, or a pharmaceutically acceptable salt thereof, and one or more pharmaceutically acceptable carriers, excipients, or diluents. Such compositions are prepared in accordance with acceptable pharmaceutical procedures. Pharmaceutically acceptable carriers are those carriers that are compatible with the other ingredients in the formulation and are biologically acceptable.

The compounds of formula I may be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

In certain embodiments, a compound of formula I is provided in a disintegrating tablet formulation suitable for pediatric administration.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

In certain embodiments, a liquid pharmaceutical composition is provided wherein said composition is suitable for pediatric administration. In other embodiments, the liquid composition is a syrup or suspension.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The compounds of formula I may be administered rectally or vaginally in the form of a conventional suppository. For administration by intranasal or intrabronchial inhalation or insufflation, the compounds of formula I can be formulated into an aqueous or partially aqueous solution, which can then be utilized in the form of an aerosol. The compounds of formula I can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The therapeutically effective amount of a compound of formula I provided to a patient will vary depending upon what is being administered, the purpose of the administration, such as prophylaxis or therapy, the state of the patient, the manner of administration, or the like. In therapeutic applications, compounds of formula I are provided to a patient suffering from a condition in an amount sufficient to treat or at least partially treat the symptoms of the condition and its complications. An amount adequate to accomplish this is a “therapeutically effective amount” as described previously herein. The dosage to be used in the treatment of a specific case must be subjectively determined by the attending physician. The variables involved include the specific condition and the size, age, and response pattern of the patient. Generally, a starting dose is about 5 mg per day with gradual increase in the daily dose to about 150 mg per day, to provide the desired dosage level in the patient.

In certain embodiments, the present invention is directed to prodrugs of compounds of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975).

For a more clear understanding, and in order to illustrate the invention more clearly, specific examples thereof are set forth hereinbelow. The following examples are merely illustrative and are not to be understood as limiting the scope and underlying principles of the invention in any way. The terms HPLC and NMR designate high performance liquid chromatography and proton nuclear magnetic resonance, respectively. The term MS designates mass spectroscopy with (+) referring to the positive mode which generally gives a M+1 (or M+H) absorption where M=the molecular mass. All compounds are analyzed at least by MS and NMR. The term Boc designates t-butoxycarbonyl. Unless otherwise noted, all parts are parts by weight.

### EXAMPLES

### Example 1

### Preparation of 4-(2-Methylbenzimidazol-1-yl)benzoic acid

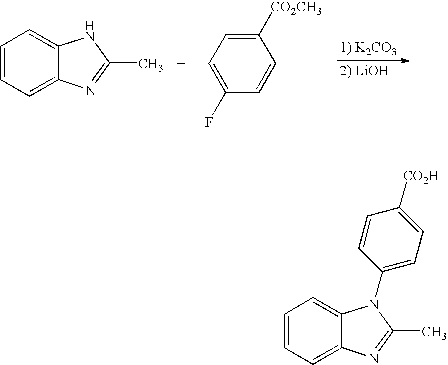

Step 1: A solution of 2-methylbenzimidazole (5.00 g, 37.68 mmol) in anhydrous methylsulfoxide in a pressure vessel at room temperature was treated with potassium carbonate (20.83 g, 150.72 mmol), stirred at room temperature for 0.5 h and treated with methyl-4-fluorobenzoate (14.62 mL, 113.03 mmol). The pressure vessel was sealed, allowed to heat at 80° C. for 72 h and cooled to room temperature. The vessel was unsealed and the reaction mixture was filtered. The filtrate was partitioned between dichloromethane and 5% aqueous citric acid. The organic phase was washed sequentially with 5% aqueous citric acid, saturated aqueous sodium bicarbonate, and brine, dried over sodium sulfate and concentrated in vacuo. The resultant residue was purified by ISCO CombiFlash® chromatography (silica, 2.5-3.5% methanol/dichloromethane) to provide methyl 4-(2-methylbenzimidazol-1-yl)benzoate as an off-white solid, 5.72 g (57%), mp 153-154° C.; MS (ES) m/z 267.1 [M+H]⁺.

Step 2: A solution of methyl 4-(2-methylbenzoimidazol-1-yl)benzoate (0.34 g, 1.26 mmol) in tetrahydrofuran was treated with lithium hydroxide solution (2.6 mL, 2.0 N) at room temperature, stirred at room temperature for 18 h and partitioned between sodium hydroxide and ethyl ether. The aqueous phase was washed with ethyl ether, acidified with aqueous hydrochloric acid to pH 1-2, treated with saturated aqueous sodium chloride, set in the refrigerator for 2 hours and filtered. The filtercake was dried under reduced pressure to give the title product as a white solid, 0.3 g (98.5%), mp 299-300° C., MS (ES) m/z 253.1 [M+H]⁺.

### Example 2

### Preparation of 3-[4-(2-Methylbenzimidazol-1-yl)benzoylamino]-(R)-pyrrolidine-1-carboxylic acid tert-butyl ester

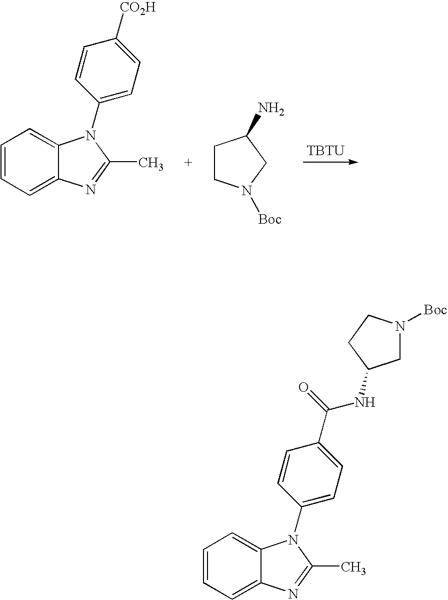

A solution of 4-(2-methylbenzoimidazol-1-yl)-benzoic acid (1.5 g, 5.95 mmol), (R)-(+)-N-Boc-3-aminopyrrolidine (1.11 mL, 6.54 mmol) and 4-methylmorpholine (3.27 mL, 29.75 mmol) in anhydrous tetrahydrofuran at 0° C. was treated with 2-(1H-benzotriazol-1-yl)-1,1,3,3-tetramethyluronium tetrafluoroborate (TBTU) (2.20 g, 6.84 mmol), allowed to warm to room temperature, stirred at room temperature for 2 h and concentrated in vacuo. The resultant residue was diluted with 5% aqueous citric acid and extracted with dichloromethane. The extracts were combined, washed sequentially with saturated aqueous sodium bicarbonate and brine, dried over anhydrous magnesium sulfate and concentrated to dryness in vacuo to provide the title product as a yellow viscous oil, 2.23 g (90%). [α]_{D}²⁵=−24° (c=1.00 in methanol); MS (ES) m/z 421 [M+H]⁺.

### Example 3

### Preparation of N-Methyl-4-(2-methyl-1H-benzimidazol-1-yl)-N—(R)-pyrrolidin-3-yl-benzamide Hydrochloride

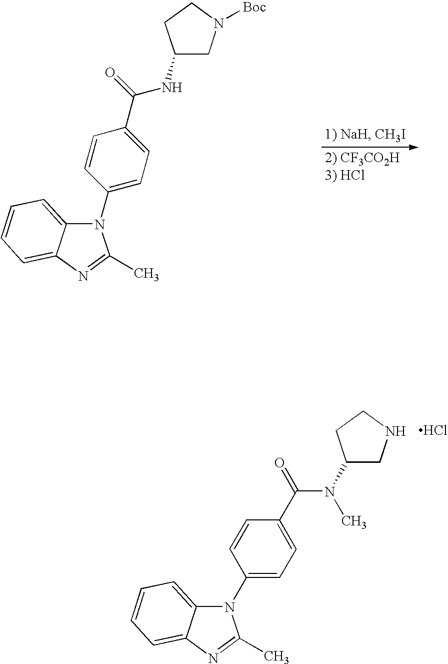

Step 1: A solution of 3-[4-(2-methylbenzoimidazol-1-yl)benzoylamino]-(R)-pyrrolidine-1-carboxylic acid tert-butyl ester (2.00 g, 4.76 mmol) in anhydrous tetrahydrofuran at 0° C. was treated slowly with sodium hydride (60% dispersion in mineral oil, 0.48 g, 11.90 mmol), stirred at 0° C. for 0.5 h, treated with iodomethane (0.90 mL, 14.27 mmol), stirred at room temperature for 18 h, quenched with 5% aqueous citric acid and extracted with ethyl acetate. The extracts were combined, washed sequentially with aqueous citric acid, saturated aqueous sodium bicarbonate and brine, dried over magnesium sulfate and concentrated in vacuo. The resultant residue was purified by ISCO CombiFlash® chromatography (silica, 1-4% methanol/dichlormethane) to provide 3-{methyl-[4-(2-methylbenzimidazol-1-yl)benzoyl]amino}-(R)-pyrrolidine-1-carboxylic acid tert-butyl ester as a yellow foam, 1.2 g (58%), [α]_{D}²⁵=+43° (c=1.00 in methanol); MS (ES) m/z 435.40 [M+H]⁺.

Step 2: A solution of 3-{methyl-[4-(2-methylbenzimidazol-1-yl)benzoyl]amino}-(R)-pyrrolidine-1-carboxylic acid tert-butyl ester (3.2 g, 7.36 mmol) in dichloromethane at room temperature was treated with trifluoroacetic acid (8 mL), stirred at room temperature for 20 h and concentrated in vacuo. The resultant residue was dispersed in sodium hydroxide and saturated aqueous sodium chloride and extracted with methylene chloride until no product was detected in the aqueous phase by thin layer chromatography. The extracts were combined, washed with saturated aqueous sodium chloride, dried over sodium sulfate and concentrated in vacuo. This residue was purified by ISCO CombiFlash® chromatography (silica, 0.2% ammonium hydroxide, 5% methanol/dichloromethane) to afford N-methyl-4-(2-methyl-benzoimidazol-1-yl)-N—(R)-pyrrolidin-3-yl-benzamide as a white foam, 2.17 g (88.2%). The foam was dissolved in ethyl acetate, treated with ethereal HCl, allowed to stand at 10-25° C. and filtered. The filtercake was dried to afford the title product as a white solid, mp 171-172° C.; MS (ES) m/z 335.1 [M+H]⁺.

### Example 4

### Preparation of N-[(3R)-1-Isobutylpyrrolidin-3-yl]-N-methyl-4-(2-methyl-1H-benzimidazol-1-yl)benzamide Hydrochloride

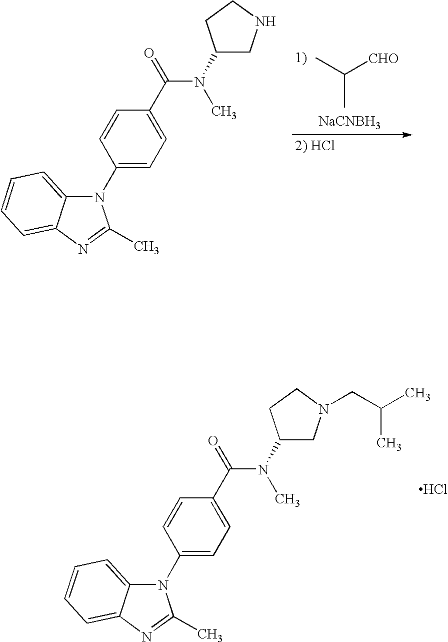

A solution of N-methyl-4-(2-methyl-1H-benzimidazol-1-yl)-N—(R)-pyrrolidin-3-y-benzamide (0.1 g, 0.3 mmol), isobutylaldehyde (0.033 mL, 0.36 mmol) and acetic acid (0.07 mL, 0.6 mmol) in methanol at 0° C. was treated with sodium cyanoborohydride (0.028 g, 0.45 mmol), allowed to warm to room temperature, stirred at room temperature for 3 h, quenched by the addition of saturated aqueous sodium bicarbonate (5 mL), aqueous sodium hydroxide (2 mL, 2.5 N), and aqueous saturated sodium chloride (2 mL) and extracted with dichloromethane. The extracts were combined, washed with saturated aqueous sodium chloride, dried over sodium sulfate and concentrated in vacuo. The resultant residue was purified by ISCO CombiFlash® chromatography (silica, 3-5% methanol/dichloromethane) to give the free amine of the title product as a colorless foam. The foam was dissolved in ethyl acetate, treated with ethereal HCl, allowed to stand at 10-25° C. and filtered. The filtercake was dried to afford the title product as a white solid, 0.082 g (64%), mp 189-190° C.; [α]_{D}²⁵=−7° (c=1.00 in methanol); identified by NMR and mass spectral analyses. MS (ES) m/z 391.2 [M+H]⁺.

### Example 5

### Preparation of N-[(3R)-1-Cyclohexylpyrrolidin-3-yl]-N-methyl-4-(2-methyl-1H-benzimidazol-1-yl)benzamide Hydrochloride

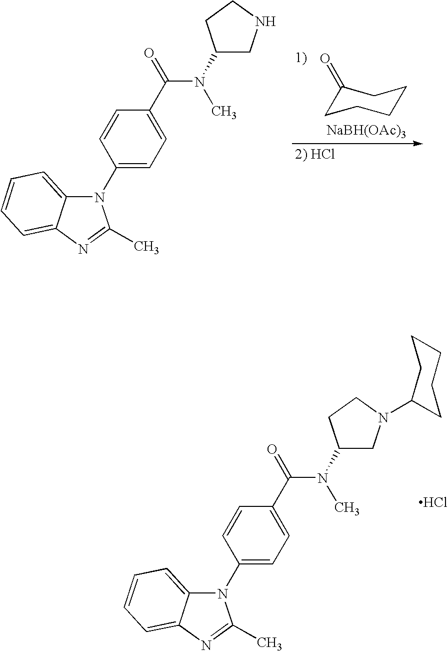

A solution of N-methyl-4-(2-methylbenzimidazol-1-yl)-N—(R)-pyrrolidin-3-yl-benzamide (0.1 g, 0.3 mmol), cyclohexanone (0.037 mL, 0.36 mmol) and acetic acid (0.07 mL, 0.6 mmol) in 1,2-dichloroethane at 0° C. was treated with sodium triacetoxyborohydride (0.095 g, 0.45 mmol), allowed to warm to room temperature, stirred at room temperature for 3 h, quenched with saturated aqueous sodium bicarbonate (5 mL), sodium hydroxide (2 mL, 2.5 N), and aqueous saturated sodium chloride (2 mL) and extracted with dichloromethane. The extracts were combined, washed with aqueous saturated sodium chloride, dried over sodium sulfate and concentrated in vacuo. The resultant residue was purified by ISCO CombiFlash® chromatography (silica, 2.5-4% methanol/dichloromethane) to provide the free amine of the title product as a colorless foam. The foam was dissolved in ethyl acetate, treated with ethereal HCl, allowed to stand at 10-25° C. and filtered. The filtercake was dried to afford the title product as a white solid, 0.11 g (81%), mp 193-194° C.; identified by NMR and mass spectral analyses. [α]_{D}²⁵=+16° (c=1.00 in methanol). MS (ES) m/z 417.2 [M+H]⁺. HRMS: calcd for C₂₆H₃₂N₄O+H⁺, 417.26489; found (ESI, [M+H]⁺ Obs'd), 417.2649.

### Examples 6-18

### Preparation of N-[(3R)-1-Substituted-pyrrolidin-3-yl]-N-methyl-4-(2-methyl-1H-benzimidazol-1-yl)benzamide Hydrochloride Compounds

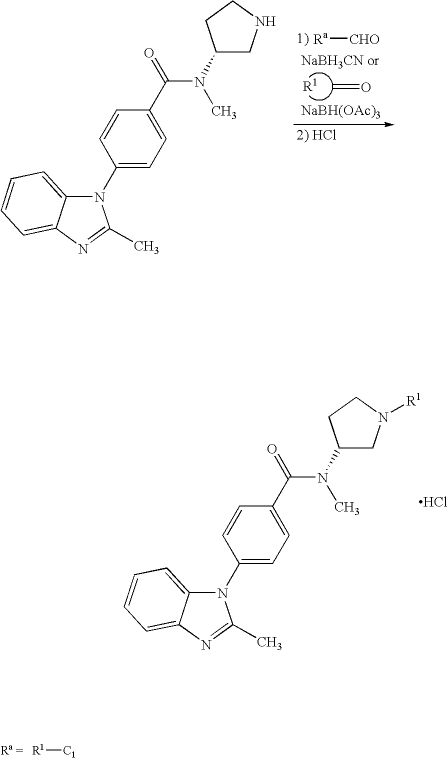

Using essentially the same procedures described in Examples 4 and 5 and employing the desired aldehyde or ketone, the compounds shown in Table I were obtained and identified by NMR and mass spectral analyses.

TABLE I

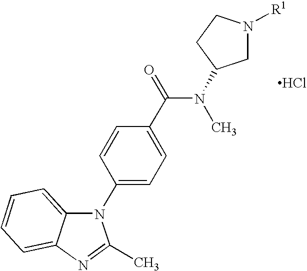

| Ex. No. | R¹ | mp ° C. | [M + H] | [α]_{D}^{25*} |
|---|---|---|---|---|
| 6 | ethyl | 171-173 | 363.1 | +6 |
| 7 | propyl | 180-182 | 377.1 | +3 |
| 8 | cyclopropylmethyl | 185-186 | 389.1 | +4 |
| 9 | cyclopentylmethyl | 190-192 | 417.2 | +3 |
| 10 | cyclohexylmethyl | 184-185 | 431.2 | −2 |
| 11 | methyl | 178-180 | 349.2 | — |
| 12 | isopropyl | 181-183 | 377.1 | +9 |
| 13 | cyclobutyl | 175-176 | 389.1 | +11 |
| 14 | cyclopentyl | 186-187 | 403.1 | +12 |
| 14 | cyclopentyl | 186-187 | 403.1 | +12 |
| 15 | cycloheptyl | 180-182 | 431.2 | +8 |
| 16 | tetrahydropyran-4-yl | 200-202 | 419.1 | +14 |
| 17 | bicyclo[2.2.1]hept-2-yl | 205-207 | 429.2 | +3 |
| 18 | adamantan-2-yl | 257-259 | 469.3 | −10 |

*1.00% solution in methanol

### Example 19

### Preparation of N-Methyl-4-(2-methylbenzimidazol-1-yl)-N—(S)-pyrrolidin-3-yl-benzamide Hydrochloride

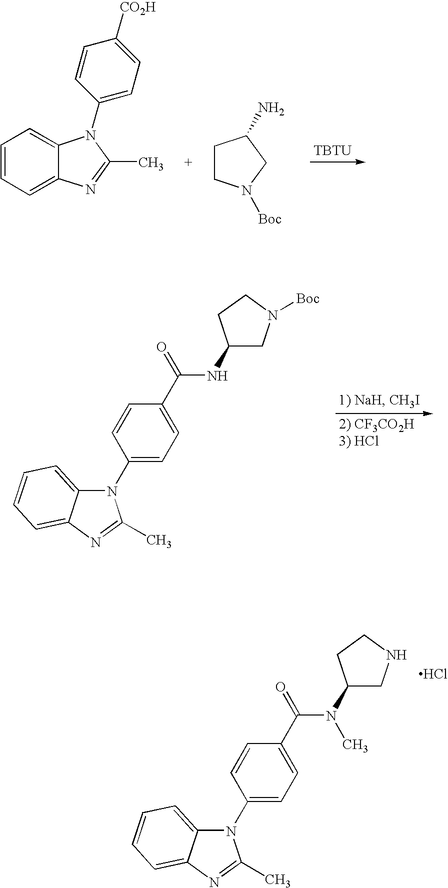

### Step 1: (S)-tert-butyl 3-(4-(2-methyl-1H-benzo[d]imidazol-1-yl)benzamido)-pyrrolidine-1-carboxylate

Using essentially the same procedure described in Example 2 and employing (S)-(−)-N-Boc-3-aminopyrrolidine as starting material, the title compound was obtained as a white foam. [α]_{D}²⁵=+30° (c=1% solution in Methanol); MS (ES) m/z 421.2 [M+H]⁺.

### Step 2: 3-{Methyl-[4-(2-methyl-benzoimidazol-1-yl)-benzoyl]-amino}-(S)-pyrrolidine-1-carboxylic acid tert-butyl ester

Using essentially the same procedure described in Example 3 (step 1) and employing (S)-tert-butyl 3-(4-(2-methyl-1H-benzo[d]imidazol-1-yl)benzamido)-pyrrolidine-1-carboxylate as the starting material, the title product was obtained as a yellow foam. [α]_{D}²⁵=−51° (c=1% solution in Methanol); MS (ES) m/z 435.2 [M+H]⁺.

### Step 3: N-Methyl-4-(2-methyl-benzoimidazol-1-yl)-N—(S)-pyrrolidin-3-yl-benzamide

Using essentially the same procedure described in Example 3 (step 2) and employing 3-{methyl-[4-(2-methyl-benzoimidazol-1-yl)-benzoyl]-amino}-(S)-pyrrolidine-1-carboxylic acid tert-butyl ester as the starting material, the title product was obtained as a white solid, mp 130-132° C.; MS (ES) m/z 335.2 [M+H]⁺.

### Examples 20-29

### Preparation of N-[(3S)-1-Substituted-pyrrolidin-3-yl]-N-methyl-4-(2-methyl-1H-benzimidazol-1-yl)benzamide Hydrochloride Compounds

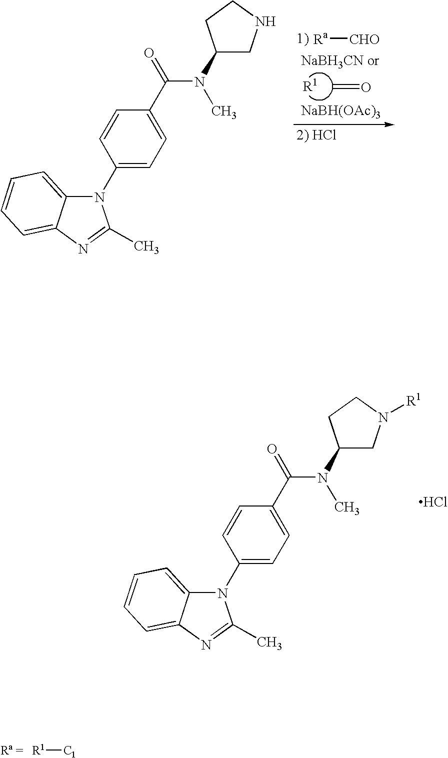

Using essentially the same procedures described in Examples 4 and 5 and employing N-methyl-4-(2-methylbenzimidazol-1-yl)-N—(S)-pyrrolidin-3-yl-benzamide_and the desired aldehyde or ketone, the compounds shown in Table II were obtained and identified by NMR and mass spectral analyses.

TABLE II

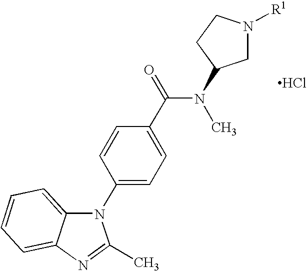

| Ex. No. | R¹ | mp ° C. | [M + H] | [α]_{D}^{25*} |
|---|---|---|---|---|
| 20 | isopropyl | 190-192 | 377.2 | −9 |
| 21 | cyclobutyl | 184-185 | 389.2 | −11 |
| 22 | cyclopentyl | 165-166 | 403.2 | −14 |
| 23 | cyclohexyl | 170-172 | 417.3 | −14 |
| 24 | 3-methylcyclopentyl | 173-175 | 417.3 | −11 |
| 25 | (R)-3-methylcyclopentyl | 175-177 | 417.3 | −11 |
| 26 | 2-methylcyclohexyl | 195-196 | 431.3 | — |
| 27 | (R)-3-methylcyclohexyl | 187-189 | 431.3 | −7 |
| 28 | 3-methylcyclohexyl | 181-183 | 431.3 | −10 |
| 29 | cyclopropylmethyl | 179-180 | 389.2 | — |

*1.00% solution in methanol

### Example 30

### Preparation of N-Methyl-4-(2-methylbenzimidazol-1-yl)-N-piperidin-4-yl-benzamide Hydrochloride

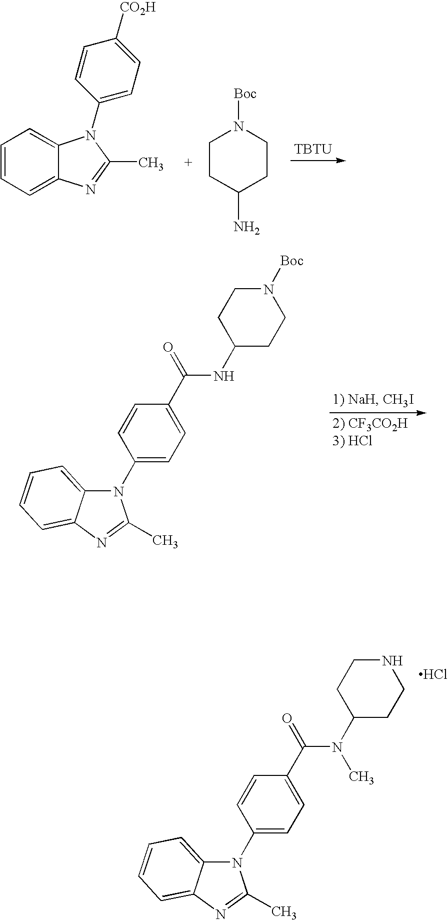

### Step 1: tert-butyl 4-(4-(2-methyl-1H-benzo[d]imidazol-1-yl)benzamido)-piperidine-1-carboxylate

Using essentially the same procedure described in Example 2 and employing t-butyl 4-aminopiperidine-1-carboxylate as starting material, the title product was obtained as an off-white foam. MS (ES) m/z 435.2 [M+H]⁺.

### Step 2: N-methyl-4-(2-methyl-1H-benzo[d]imidazol-1-yl)-N-(piperidin-4-yl)benzamide

Using essentially the same procedure described in Example 3 (step 1) and employing tert-butyl 4-(4-(2-methyl-1H-benzo[d]imidazol-1-yl)benzamido)-piperidine-1-carboxylate as the starting material, the title product was obtained as a yellow foam. MS (ES) m/z 449.3 [M+H]⁺.

### Step 3: N-Methyl-4-(2-methyl-benzoimidazol-1-yl)-N-piperidin-4-yl-benzamide

Using essentially the same procedure described in Example 3 (step 2) and employing N-methyl-4-(2-methyl-1H-benzo[d]imidazol-1-yl)-N-(piperidin-4-yl)benzamide as the starting material, the title product was obtained as a yellow solid, mp 219-221° C.; identified by NMR and mass spectral analyses. MS (ES) m/z 349.2 [M+H]⁺.

### Examples 31-34

### Preparation of N-[1-Substituted-piperidin-4-yl]-N-methyl-4-(2-methyl-1H-benzimidazol-1-yl)benzamide Hydrochloride Compounds

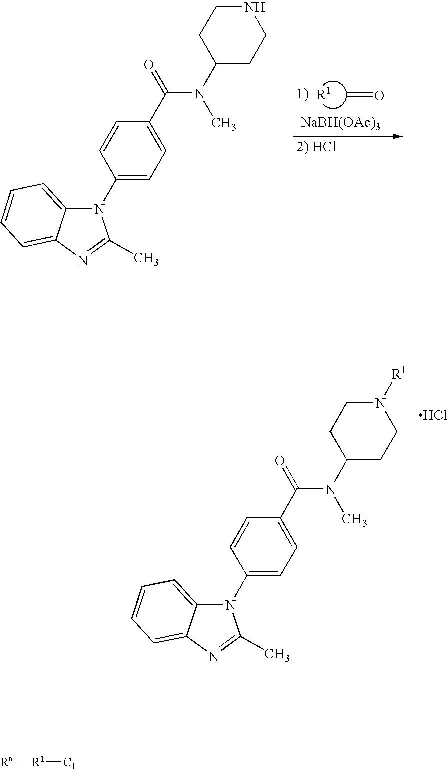

Using essentially the same procedure described in Example 5 and employing N-methyl-4-(2-methylbenzimidazol-1-yl)-N-piperidin-4-yl-benzamide and the desired ketone, the compounds shown in Table III were obtained and identified by NMR and mass spectral analyses.

TABLE III

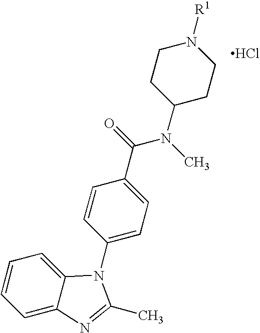

| Ex. No. | R¹ | mp ° C. | [M + H] |
|---|---|---|---|
| 31 | isopropyl | 282-284 | 391.2 |
| 32 | cyclopentyl | 270 (dec) | 417.2 |
| 33 | cyclohexyl | 285 (dec) | 431.2 |
| 34 | cyclobutyl | 280 (dec) | 403.2 |

### Example 35

### Preparation of N-Methyl-4-[(2-methylbenzimidazol-1-yl)methyl]-N—(R)-pyrrolidin-3-yl-benzamide Hydrochloride

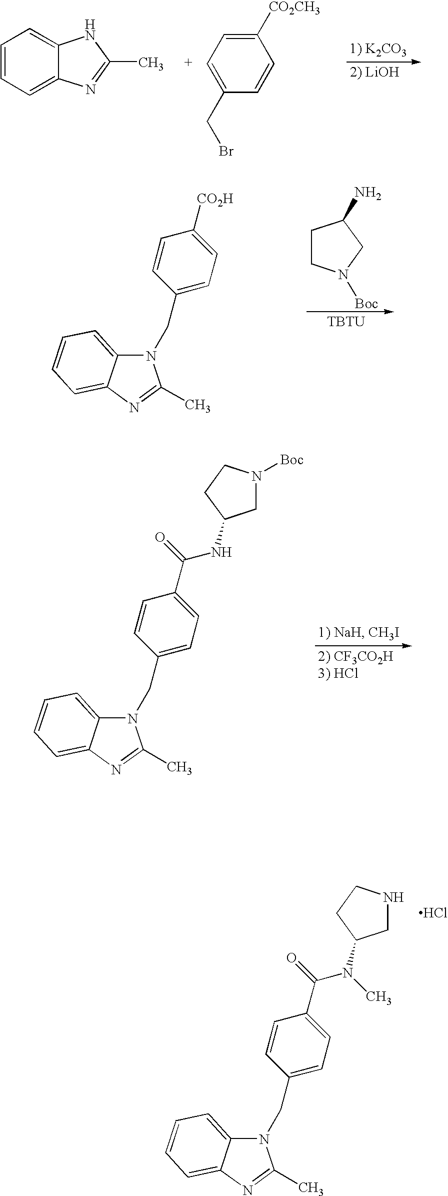

### Step 1: 4-(2-Methyl-benzoimidazol-1-ylmethyl)-benzoic acid methyl ester

Using essentially the same procedure described in Example 1 (step 1) and employing methyl 4-bromomethylbenzoate as starting material, the title product was obtained as a yellow solid, mp 100-101° C.; MS (ES) M/ZI m/z 281.1 [M+H]⁺.

### Step 2: 4-(2-Methyl-benzoimidazol-1-ylmethyl)-benzoic acid

Using essentially the same procedure described in Example 1 (step 2) and employing 4-(2-Methyl-benzoimidazol-1-ylmethyl)-benzoic acid methyl ester as starting material, the title product was obtained as a white solid. mp 300° C. decomposed; MS (ES) m/z 267.2[M+H]⁺.

### Step 3: 3-[4-(2-Methyl-benzoimidazol-1-ylmethyl)-benzoylamino]-(R)-pyrrolidine-1-carboxylic acid tert-butyl ester

Using essentially the same procedure described in Example 2 and employing 4-(2-methyl-benzoimidazol-1-ylmethyl)-benzoic acid as starting material, the title product was obtained as a yellow solid. [α]_{D}²⁵=−22° (c=1% solution in Methanol); MS (ES) m/z 435.2 [M+H]⁺.

### Step 4: 3-{Methyl-[4-(2-methyl-benzoimidazol-1-ylmethyl)-benzoyl]-amino}-(R)-pyrrolidine-1-carboxylic acid tert-butyl ester

Using essentially the same procedure described in Example 3 (step 1) and employing 3-[4-(2-methyl-benzoimidazol-1-ylmethyl)-benzoylamino]-(R)-pyrrolidine-1-carboxylic acid tert-butyl ester as starting material, the title product was obtained as a yellow foam. [α]_{D}²⁵=−2° (c=1% solution in Methanol); MS (ES) m/z 449.2 [M+H]⁺.

### Step 5: N-Methyl-4-(2-methyl-benzoimidazol-1-ylmethyl)-N—(R)-pyrrolidin-3-yl-benzamide

Using essentially the same procedure described in Example 3 (step 2) and employing 3-{methyl-[4-(2-methyl-benzoimidazol-1-ylmethyl)-benzoyl]-amino}-(R)-pyrrolidine-1-carboxylic acid tert-butyl ester as starting material, the title product was obtained as a white solid, mp 126-128° C.; [α]_{D}²⁵=−0.69° (c=7 mg in 0.8 mL Methanol); MS (ES) m/z 349.1 [M+H]⁺; HRMS: calcd for C₂₁H₂₄N₄O+H⁺, 349.20229; found (ESI, [M+H]⁺ Obs'd), 349.2025.

### Example 36

### Preparation of (R)-4-((1H-benzo[d]imidazol-1-yl)methyl)-N-methyl-N-(pyrrolidin-3-yl)benzamide Hydrochloride

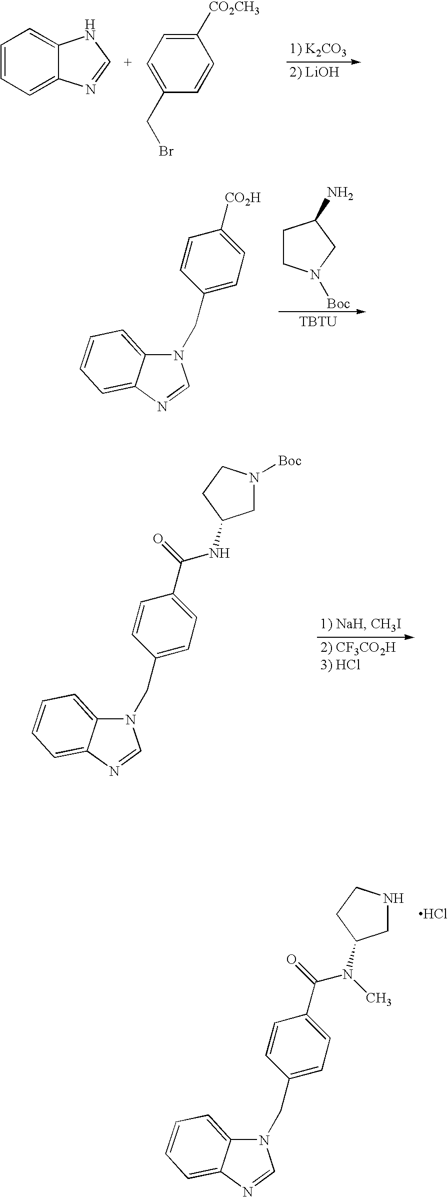

### Step 1: Methyl-4-((1H-benzo[d]imidazol-1-yl)methyl)benzoate

Using essentially the same procedure described in Example 1 (step 1) and employing benzimidazole as starting material, the title product was obtained as a light yellow solid, mp 94-95° C., MS (ES) m/z 267.1 [M+H]⁺.

### Step 2: 4-((1H-benzo[d]imidazol-1-yl)methyl)benzoic acid

Using essentially the same procedure described in Example 1 (step 2) and employing methyl-4-((1H-benzo[d]imidazol-1-yl)methyl)benzoate as starting material, the title product was obtained as a white solid, mp 94-95° C., MS (ES) m/z 253.1 [M+H]⁺.

### Step 3: (R)-tert-butyl 3-(4-((1H-benzo[d]imidazol-1-yl)methyl)benzamido)-pyrrolidine-1-carboxylate

Using essentially the same procedure described in Example 2 and employing 4-((1H-benzo[d]imidazol-1-yl)methyl)benzoic acid as starting material, the title product was obtained, [α]_{D}²⁵=−23.8° (c=7 mg in 0.8 mL Methanol); MS (ES) m/z 421.2[M+H]⁺;

### Step 4: 3-[(4-Benzoimidazol-1-ylmethyl-benzoyl)-methyl-amino]-(R)-pyrrolidine-1-carboxylic acid tert-butyl ester

Using essentially the same procedure described in Example 3 (step 1) and employing (R)-tert-butyl 3-(4-((1H-benzo[d]imidazol-1-yl)methyl)benzamido)-pyrrolidine-1-carboxylate as the starting material, the titled product was obtained as a white foam, [α]_{D}²⁵=−1.0° (c=7 mg in 0.8 mL Methanol); MS (ES) m/z 435.2 [M+H]⁺;

### Step 5: (R)-4-((1H-benzo[d]imidazol-1-yl)methyl)-N-methyl-N-(pyrrolidin-3-yl)benzamide

Using essentially the same procedure described in Example 3 (step 2) and employing 3-[(4-Benzoimidazol-1-ylmethyl-benzoyl)-methyl-amino]-(R)-pyrrolidine-1-carboxylic acid tert-butyl ester as starting material, the title product was obtained as a white solid, mp 150-152° C.; [α]_{D}²⁵=−0.6° (c=7 mg in 0.8 mL methanol); MS (ES) m/z 335.2 [M+H]⁺;

### Examples 37-43

### Preparation of N-[(3R)-1-Substituted-pyrrolidin-3-yl]-N-methyl-4-[(2-methyl-1H-benzimidazol-1-yl)methyl]benzamide Hydrochloride Compounds and N-[(3R)-1-Substituted-pyrrolidin-3-yl]-N-methyl-4-[(1H-benzimidazol-1-yl)methyl]-benzamide Hydrochloride Compounds

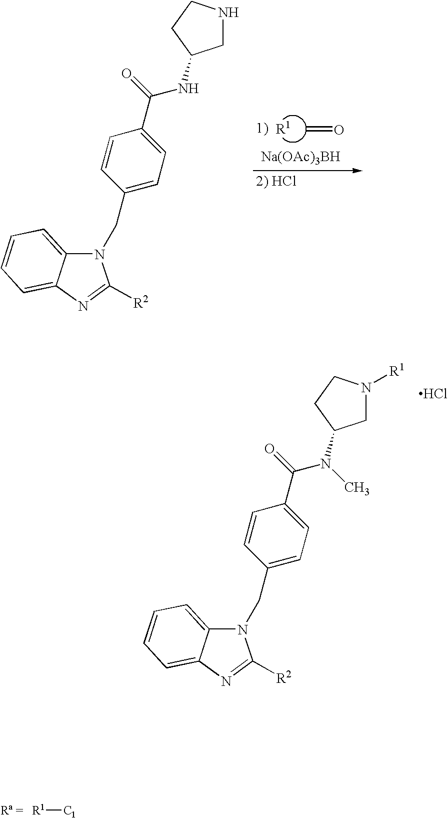

Using essentially the same procedure described in Example 5 and employing the appropriate benzamide substrate and ketone, the compounds shown in Table IV were obtained and identified by NMR and mass spectral analyses.

TABLE IV

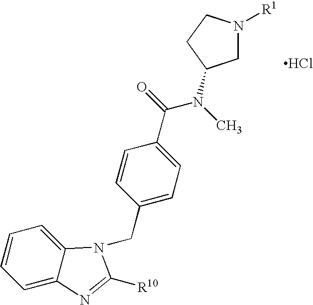

| Ex. No. | R¹ | R¹⁰ | mp ° C. | [M + H] | [α]_{D}^{25*} |
|---|---|---|---|---|---|
| 37 | isopropyl | CH₃ | 163-164 | 391.2 | +4.6* |
| 38 | cyclobutyl | CH₃ | 172-174 | 403.2 | +6.63* |
| 39 | cyclopentyl | CH₃ | 179-180 | 417.3 | +7.4* |
| 40 | cyclohexyl | CH₃ | 188-190 | 431.3 | +9.2* |
| 41 | isopropyl | H | 167-169 | 377.3 | +5.2* |
| 42 | cyclobutyl | H | 154-155 | 389.3 | +7.0* |
| 43 | cyclopentyl | H | 164-165 | 403.3 | +8.0* |

*1.00% solution in methanol

### Example 44

### Preparation of N-methyl-4-((2-methyl-1H-benzo[d]imidazol-1-yl)methyl)-N-(piperidin-4-yl)benzamide

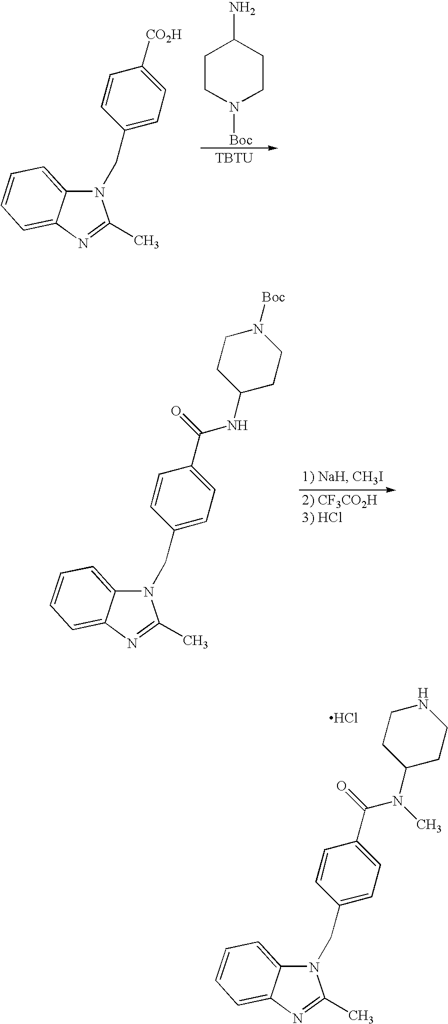

### Step 1: tert-butyl 4-(4-((2-methyl-1H-benzo[d]imidazol-1-yl)methyl)benzamido)-piperidine-1-carboxylate

Using essentially the same procedure described in Example 2 and employing tert-butyl 4-aminopiperidine-1-carboxylate as starting material, the title product was obtained as a yellow solid, mp 77-79° C., MS (ES) m/z 449.3 [M+H]⁺;

### Step 2: tert-butyl 4-(N-methyl-4-((2-methyl-1H-benzo[d]imidazol-1-yl)methyl)benzamido)piperidine-1-carboxylate

Using essentially the same procedure described in Example 3 (step 1) and employing tert-butyl 4-(4-((2-methyl-1H-benzo[d]imidazol-1-yl)methyl)benzamido)piperidine-1-carboxylate as starting material, the title product was obtained as a white foam, MS (ES) m/z 463.3 [M+H]⁺.

### Step 3: N-methyl-4-((2-methyl-1H-benzo[d]imidazol-1-yl)methyl)-N-(piperidin-4-yl)benzamide

Using essentially the same procedure described in Example 3 (Step 2) and employing N-methyl-4-((2-methyl-1H-benzo[d]imidazol-1-yl)methyl)-N-(piperidin-4-yl)benzamide as starting material, the title product was obtained as a white solid, mp 196-198° C., MS (ES) m/z 363.2 [M+H]⁺.

### Example 45

### Preparation of 4-((1H-benzo[d]imidazol-1-yl)methyl)-n-methyl-N-(piperidin-4-yl)benzamide

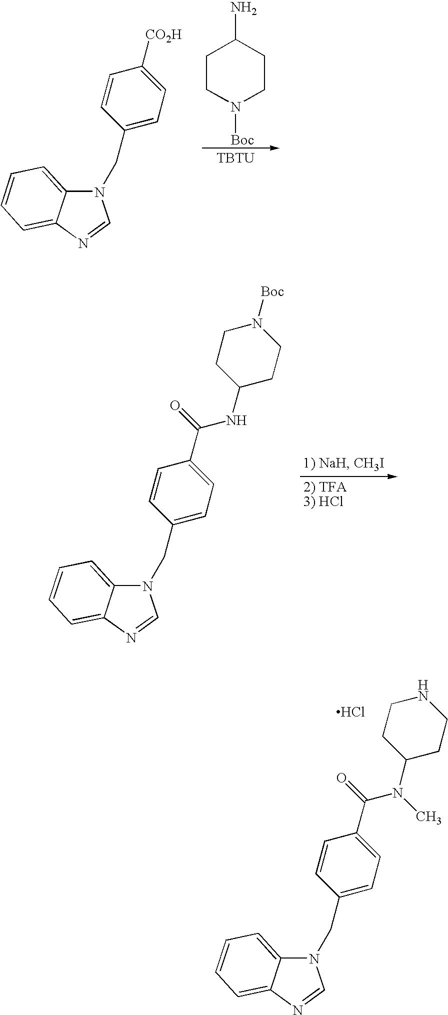

### Step 1: tert-butyl 4-(4-((1H-benzo[d]imidazol-1-yl)methyl)benzamido)piperidine-1-carboxylate

Using essentially the same procedure described in Example 2 and employing 4-((1H-benzo[d]imidazol-1-yl)methyl)benzoic acid as starting material, the title product was obtained as a yellow foam, MS (ES) m/z 435.3 [M+H]⁺.

### Step 2: tert-butyl 4-(4-((1H-benzo[d]imidazol-1-yl)methyl)-N-methylbenzamido)piperidine-1-carboxylate

Using essentially the same procedure described in Example 3 (step 1) and employing tert-butyl 4-(4-((1H-benzo[d]imidazol-1-yl)methyl)benzamido)piperidine-1-carboxylate as starting material, the title product was obtained as a white foam, MS (ES) m/z 449.3 [M+H]⁺.

### Step 3: 4-((1H-benzo[d]imidazol-1-yl)methyl)-N-methyl-N-(piperidin-4-yl)benzamide

Using essentially the same procedure described in Example 3 (Step 2) and employing tert-butyl 4-(4-((1H-benzo[d]imidazol-1-yl)methyl)-N-methylbenzamido)piperidine-1-carboxylate as starting material, the title product was obtained as a white solid, mp 199-201° C., MS (ES) m/z 349.1 [M+H]⁺.

### Examples 46-50

### Preparation of 4-((1H-benzo[d]imidazol-1-yl)methyl)-N-(1-substituted piperidin-4-yl)-N-methylbenzamide hydrochloride compounds and N-(1-substituted piperidin-4-yl)-N-methyl-4-((2-methyl-1H-benzo[d]imidazol-1-yl)methyl)-benzamide hydrochloride compounds

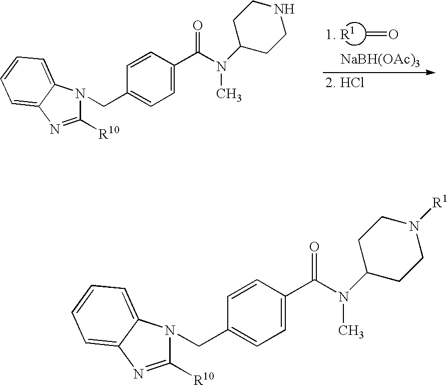

Using essentially the same procedure described in Example 5 and employing the desired ketone, the compounds shown in Table V were obtained and identified by NMR and mass spectral analyses.

TABLE V

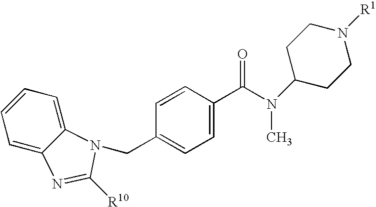

| Ex. No. | R¹ | R¹⁰ | mp ° C. | [M + H] |
|---|---|---|---|---|
| 46 | i-propyl | CH₃ | 200-202 | 405.3 |
| 47 | cyclobutyl | CH₃ | 280 (dec) | 417.3 |
| 48 | cyclopentyl | CH₃ | 280 (dec) | 431.3 |
| 49 | i-propyl | H | 185-187 | 391.2 |
| 50 | cyclobutyl | H | 238-240 | 403.1 |
| 51 | cyclopentyl | H | 248-250 | 417.2 |

### Example 52

### Preparation of 4-(5-Cyano-2-methyl-benzoimidazol-1-yl)-benzoic acid

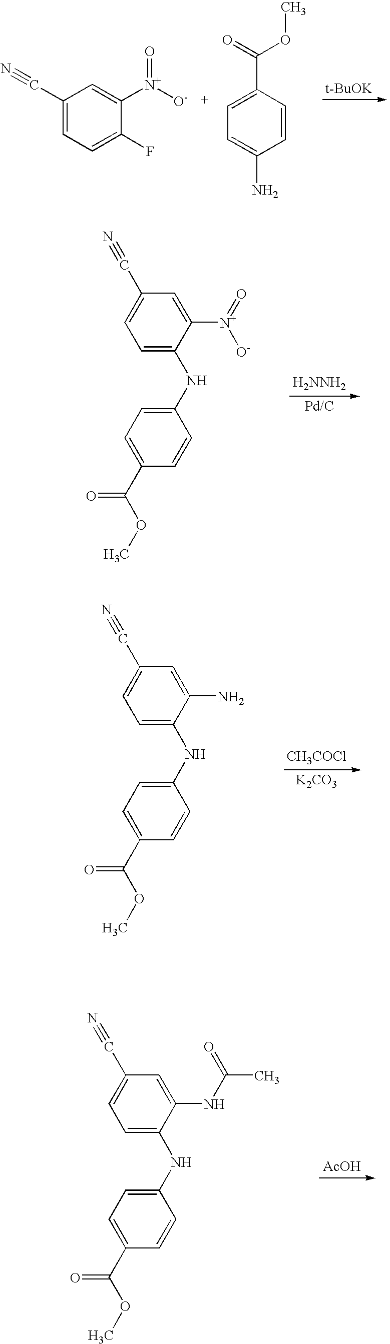

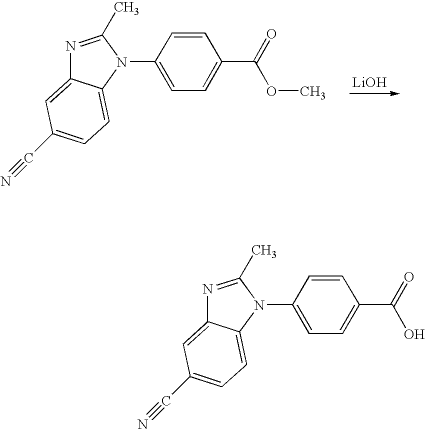

Step 1: To a solution of 4-fluoro-3-nitrobenzonitrile (2 g, 12 mmol) and methyl-4-aminobenzoate (1.91 g, 12.6 mmol) in anhydrous methylsulfoxide at 0° C. was added potassium t-butoxide (3.1 g, 26.4 mmol). The reaction mixture was warmed to room temperature, and stirred at room temperature for 4 hours, quenched with 5% citric acid. The brown solid was filtered and washed with CH₂Cl₂ (3×100 mL). The filtrate was partitioned between dichloromethane and 5% aqueous citric acid. The aqueous layer was washed with dichloromethane. The organic layers were combined and washed with saturated aqueous NaHCO₃ solution, brine, dried over sodium sulfate and concentrated in vacuo. The resultant residue was purified by ISCO ComiFlash® chromatography (silica, CH₂Cl₂) to provide 1.76 g (49%) of 4-(4-cyano-2-nitro-phenylamino)-benzoic acid methyl ester as an orange oil, MS (ES) m/z 298.3 [M+H]⁺.

Step 2: To a solution of 4-(4-cyano-2-nitro-phenylamino)-benzoic acid methyl ester (0.36 g, 1.21 mmol) and hydrazine (0.24 mL, 4.84 mmol) in ethanol was added palladium on carbon (0.04 g, 10%), and the reaction mixture was allowed to reflux for 3 hours. The palladium was filtered through the pad of celite. The filtrate was concentrated in vacuo. The residue was purified by ISCO ComiFlash® chromatography (silica, 15% ethyl acetate/CH₂Cl₂) to give 0.161 g (50%) of 4-(2-amino-4-cyano-phenylamino)-benzoic acid methyl ester as a yellow solid, mp 164-165° C. MS (ES) m/z 268.2 [M+H]⁺.

Step 3: To a solution of 4-(2-amino-4-cyano-phenylamino)-benzoic acid methyl ester (0.5 g, 1.87 mmol) at 0° C. was added acetyl chloride (0.2 mL, 2.81 mmol), K₂CO₃ (1.55 g, 11.22 mmol, 325 mesh). The reaction mixture was stirred in a water bath for 3 hours. The solid was filtered through a pad of celite. The filtrate was partitioned between ethyl acetate and water. The organic solution was washed with 5% citric acid, saturated aqueous NaHCO₃ solution, and brine; dried over sodium sulfate. The organic layers were concentrated in vacuo, then set in the refrigerator overnight. The precipitate was filtered and the filtercake was dried under reduced pressure to give 0.5 g (86%) of 4-(2-acetylamino-4-cyano-phenylamino)-benzoic acid methyl ester as an off-white solid, mp 231-232° C. MS (ES) m/z 310.2 [M+H]⁺.

Step 4: A solution of 4-(2-acetylamino-4-cyano-phenylamino)-benzoic acid methyl ester (0.15 g, 0.485 mmol) in acetic acid (10 mL) was refluxed for 4 hours, and cooled to room temperature. Brine (5 mL) was added. The reaction mixture was partitioned between methylene chloride (CH₂Cl₂) and water. The aqueous layer was extracted with CH₂Cl₂ (3×100 mL). The organic layers were combined and washed with 5% NaHCO₃ solution and brine, dried with Na₂SO₄. The solvent was removed in vacuo. The crude solid was recrystallized from 20% ethylacetate/hexane. The solid was filtered and the filtercake was dried under reduced pressure to give 0.124 g (88%) of 4-(5-cyano-2-methyl-benzoimidazol-1-yl)-benzoic acid methyl ester as a white solid, mp 179-181° C. MS (ES) m/z 292.0 [M+H]⁺.

Step 5: To a solution of 4-(5-cyano-2-methyl-benzoimidazol-1-yl)-benzoic acid methyl ester (3.25 g, 11.16 mmol) in tetrahydrofuran (40 ml) at room temperature was added aqueous LiOH solution (11.2 mL, 2 N), and ther reaction mixture was stirred at room temperature for 17 hours and then partitioned between aqueous NaOH solution (2.5 N) and ethyl ether. The aqueous phase was washed with ethyl ether and acidified with aqueous HCl to pH 1-2, treated with brine, set in the refrigerator for 4 hours and filtered. The filtercake was dried under reduced pressure to give the title product 2.28 g (94%) as a white solid, mp 300° C. (dec). MS (ES) m/z 278.1 [M+H]

### Example 53

### Preparation of (R)-4-(5-cyano-2-methyl-benzoimidazol-1-yl)-N-methyl-N-pyrrolidin-3-yl-benzamide

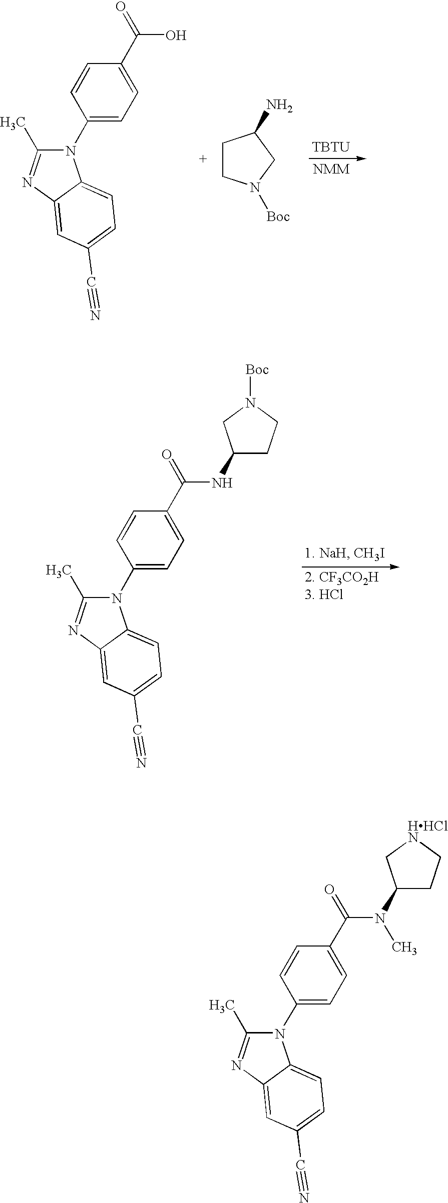

### Step 1: (R)-tert-butyl 3-(4-(5-cyano-2-methyl-1H-benzo[d]imidazol-1-yl)benzamido)pyrrolidine-1-carboxylate

Using essentially the same procedure described in Example 2 and employing (R)-(−)-N-Boc-3-aminopyrrolidine as starting material, (R)-tert-butyl 3-(4-(5-cyano-2-methyl-1H-benzo[d]imidazol-1-yl)benzamido)pyrrolidine-1-carboxylate was obtained as a yellow foam, [α]_{D}²⁵=−23.6° (c=1.00 in methanol); MS (ES) m/z 446.3 [M+H]⁺.

### Step 2: 3-{[4-(5-Cyano-2-methyl-benzoimidazol-1-yl)-benzoyl]-methyl-amino}-(R)-pyrrolidine-1-carboxylic acid tert-butyl ester

Using essentially the same procedure described in Example 3 (step 1) and employing (R)-tert-butyl 3-(4-(5-cyano-2-methyl-1H-benzo[d]imidazol-1-yl)benzamido)pyrrolidine-1-carboxylate as starting material, the title product was obtained as yellow foam, [α]_{D}²⁵=+45.6° (c=1.00 in methanol); MS (ES) m/z 460.2 [M+H]⁺.

### Step 3: (R)-4-(5-cyano-2-methyl-1H-benzo[d]imidazol-1-yl)-N-methyl-N-(pyrrolidin-3-yl)benzamide

Using essentially the same procedure described in Example 3 (Step 2) and 3-{[4-(5-cyano-2-methyl-benzoimidazol-1-yl)-benzoyl]-methyl-amino}-(R)-pyrrolidine-1-carboxylic acid tert-butyl ester as the starting material, the title product was obtained as a white solid, mp 178-180° C.; [α]_{D}²⁵=+1° (c=1.00 in methanol); MS (ES) m/z 360.2 [M+H]⁺.

### Example 54-56

### Preparation of 4-(5-Cyano-2-methyl-benzoimidazol-1-yl)-N-(1-substituted-pyrrolidin-3-yl)-N-methyl-benzamide hydrochloride compounds

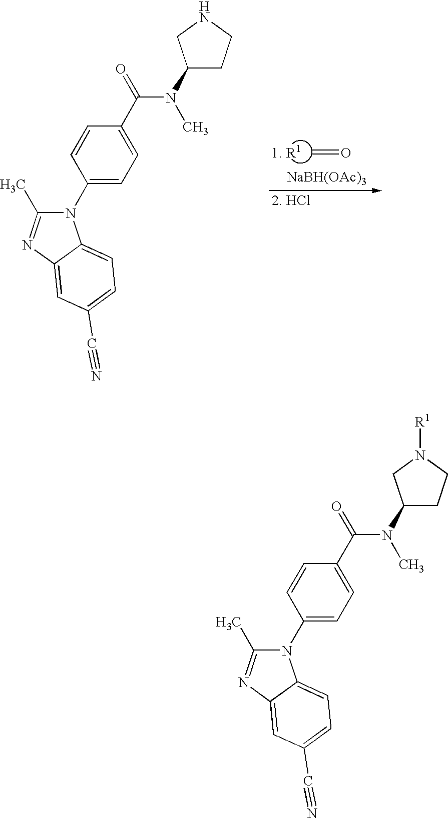

Using essentially the same procedure described in Example 5 and employing the desired ketone, the compounds shown in Table VI were obtained and identified by NMR and mass spectral analyses.

TABLE VI

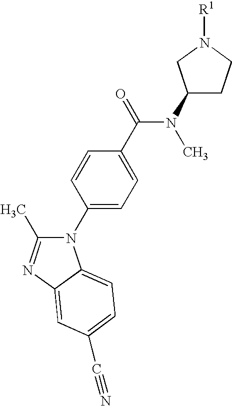

| Ex. No. | R¹ | mp ° C. | [M + H] | [α]_{D}^{25*} |
|---|---|---|---|---|
| 54 | i-propyl | 230-232 | 402.3 | +16.6 |
| 55 | cyclobutyl | 240-242 | 414.2 | +24.4 |
| 56 | cyclopentyl | 185-187 | 428.3 | +23.8 |

*1.00% solution in methanol

### Example 57-58

### Preparation of 4-Indazol-2-yl-benzoic acid methyl ester (57) 4-Indazol-1-yl-benzoic acid methyl ester (58)

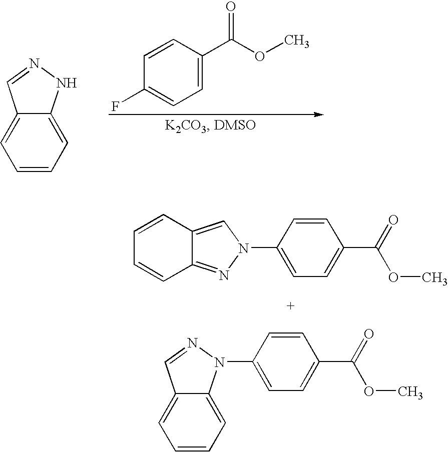

Using essentially the same procedure described in Example 1 and employing indazole as the starting material, the mixture of 4-indazol-2-yl-benzoic acid methyl ester and 4-indazol-1-yl-benzoic acid methyl ester were obtained. The mixture was separated by ISCO CombiFlash® chromatography (silica, 4-14% ethyl acetate/hexane) to provide 4-indazol-2-yl-benzoic acid methyl ester (25%) as a white solid, mp 186-187° C., MS (ES) m/z 253.0 [M+H]⁺; and 4-indazol-1-yl-benzoic acid methyl ester (39%) as a white solid, mp 80-81° C., MS (ES) m/z 253.0 [M+H]⁺.

### Example 59a-59b

### Preparation of 4-Indazol-2-yl-benzoic acid (59a) and 4-Indazol-1-yl-benzoic acid (59b)

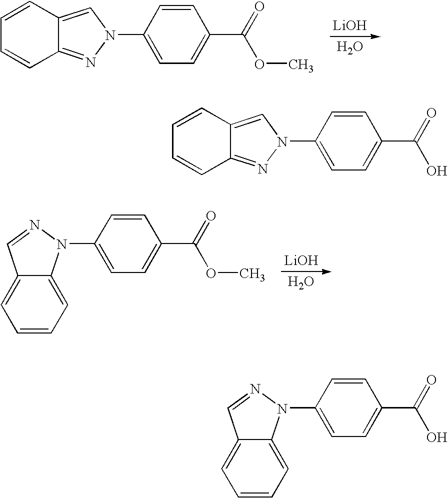

Using essential the same procedure described in Example 2 and employing 4-indazol-2-yl-benzoic acid and 4-indazol-1-yl-benzoic acid as starting material respectively, 4-indazol-2-yl-benzoic acid was obtained as a white solid, mp 286-288° C., MS (ES) m/z 237.0 [M−H]⁻; and 4-indazol-1-yl-benzoic acid was obtained as a white solid, mp 171-172° C., MS (ES) m/z 237.0 [M−H]⁻.

### Examples 60a-b

### Preparation of (R)-4-Indazol-2-yl-N-methyl-N-pyrrolidin-3-yl-benzamide (60a) and (R)-4-Indazol-1-yl-N-methyl-N-pyrrolidin-3-yl-benzamide (60b)

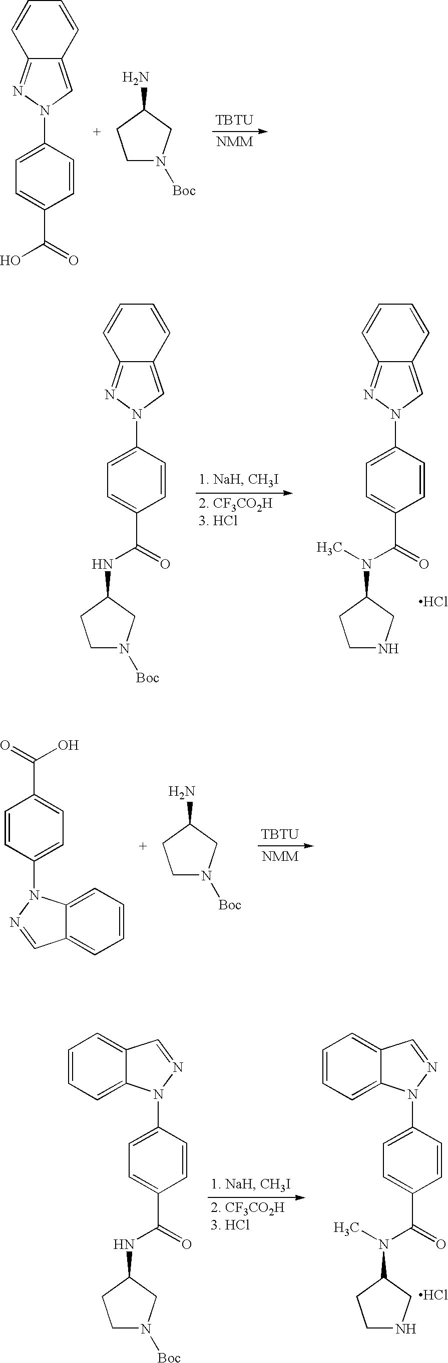

### Step 1a: (R)-tert-butyl 3-(4-(2H-indazol-2-yl)benzamido)pyrrolidine-1-carboxylate

Using essentially the same procedure described in Example 2 and employing (R)-(−)-N-Boc-3-aminopyrrolidine and 4-indazol-2-yl-benzoic acid as starting materials, (R)-tert-butyl 3-(4-(2H-indazol-2-yl)benzamido)pyrrolidine-1-carboxylate was obtained as a white solid, mp 211-212° C., [α]_{D}²⁵=−31.0° (c=1.00 in methanol), MS (ES) m/z 407.0 [M+H]⁺.

### Step 1b: (R)-tert-butyl 3-(4-(1H-indazol-1-yl)benzamido)pyrrolidine-1-carboxylate

Using essentially the same procedure described in Example 2 and employing (R)-(−)-N-Boc-3-aminopyrrolidine and 4-indazol-1-yl-benzoic acid as starting materials, (R)-tert-butyl 3-(4-(1H-indazol-1-yl)benzamido)pyrrolidine-1-carboxylate was obtained as a yellow foam, [α]_{D}²⁵=−32.0° (c=1.00 in methanol), MS (ES) m/z 407.1 [M+H]⁺.

### Step 2a: 3-[(4-Indazol-2-yl-benzoyl)-methyl-amino]-pyrrolidine-1-carboxylic acid tert-butyl ester

Using essentially the same procedure described in Example 3 (Step 1) and employing (R)-tert-butyl 3-(4-(2H-indazol-2-yl)benzamido)pyrrolidine-1-carboxylate as the starting material, 3-[(4-indazol-2-yl-benzoyl)-methyl-amino]-(R)-pyrrolidine-1-carboxylic acid tert-butyl ester was obtained as a yellow solid, mp 133-134° C., [α]_{D}²⁵=+64.0° (c=1.00 in methanol), MS (ES) m/z 421.0 [M+H]⁺.

### Step 2b: 3-[(4-Indazol-1-yl-benzoyl)-methyl-amino]-(R)-pyrrolidine-1-carboxylic acid tert-butyl ester

Using essentially the same procedure described in Example 3 (Step 1) and employing (R)-tert-butyl 3-(4-(1H-indazol-1-yl)benzamido)pyrrolidine-1-carboxylate as the starting material, 3-[(4-Indazol-1-yl-benzoyl)-methyl-amino]-(R)-pyrrolidine-1-carboxylic acid tert-butyl ester was obtained as a yellow foam, [α]_{D}²⁵=+60.0° (c=1.00 in methanol), MS (ES) m/z 421.1 [M+H]⁺.

### Step 3a: (R)-4-(2H-indazol-2-yl)-N-methyl-N-(pyrrolidin-3-yl)benzamide

Using essentially the same procedure described in Example 3 (step 2) and employing (R)-tert-butyl 3-(4-(2H-indazol-2-yl)benzamido)pyrrolidine-1-carboxylate as the starting material, the desired product 60a was obtained as an off-white solid, mp 243-245° C., [α]_{D}²⁵=−4° (c=1.00 in methanol), MS (ES) m/z 321 [M+H]⁺;

### Step 3b: (R)-4-(1H-indazol-1-yl)-N-methyl-N-(pyrrolidin-3-yl)benzamide

Using essentially the same procedure described in Example 3 (step 2) and employing (R)-tert-butyl 3-(4-(1H-indazol-1-yl)benzamido)pyrrolidine-1-carboxylate as the starting material, the desired product 60b was obtained as a yellow solid: mp 99-101° C., [α]_{D}²⁵=0° (c=1.00 in methanol); MS (ES) m/z 321.2 [M+H]⁺.

### Examples 61-66

### Preparation of (R)-4-Indazol-2-yl-N-methyl-N-substituted pyrrolidin-3-yl-benzamide hydrochloride compounds (61-63) and (R)-4-Indazol-1-yl-N-substituted methyl-N-pyrrolidin-3-yl-benzamide hydrochloride compounds (64-66)

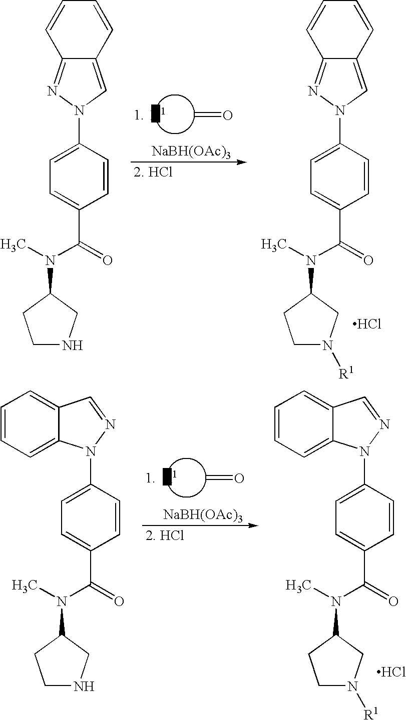

Using essentially the same procedure described in Example 5 and employing the desired ketone, the compounds shown in Table VII were obtained and identified by NMR and mass spectral analyses.

TABLE VII

| | |
|---|---|
| 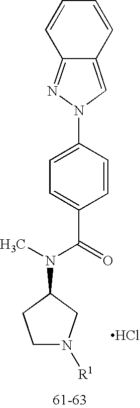 | 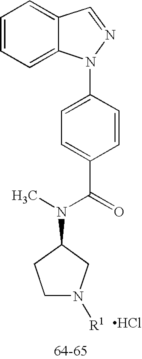 |

| Ex. No. | R¹ | mp ° C. | [M + H] | [α]_{D}^{25*} |
|---|---|---|---|---|
| 61 | i-propyl | 227-228 | 363.2 | +2.00 |
| 62 | cyclobutyl | 163-165 | 375.2 | +3.00 |
| 63 | cyclopentyl | 216-218 | 389.2 | +10.00 |
| 64 | i-propyl | 175-177 | 363.2 | +3.00 |
| 65 | cyclobutyl | 163-165 | 375.2 | +6.00 |
| 66 | cyclopentyl | 151-152 | 389.2 | +9.00 |

*1.00% solution in methanol

### Examples 67-68

### Preparation of 4-Indazol-2-yl-N-methyl-N-piperidin-3-yl-benzamide (67) and 4-Indazol-1-yl-N-methyl-N-peridin-3-yl-benzamide (68)

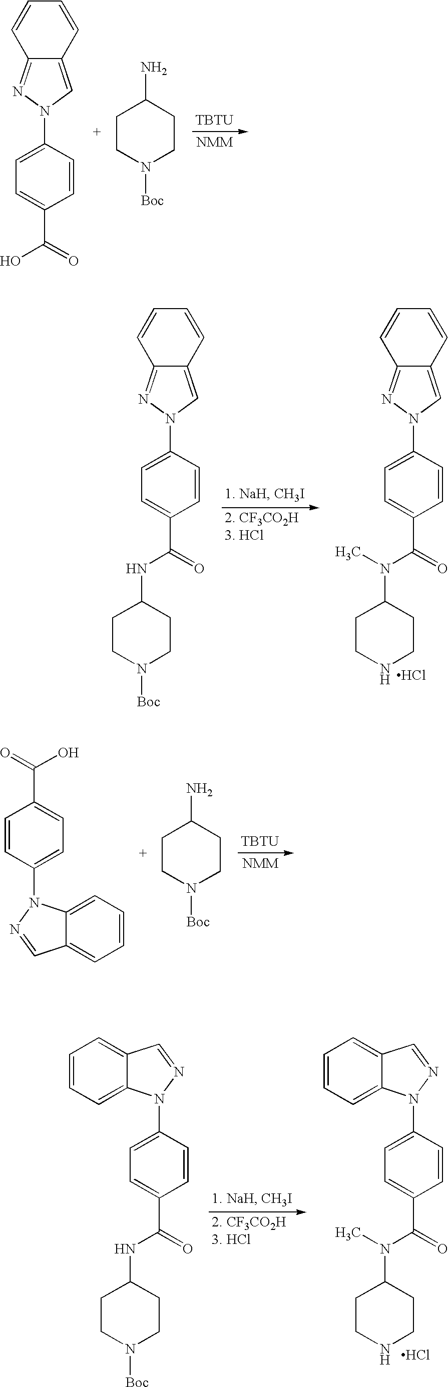

### Step 1a: tert-butyl 4-(4-(2H-indazol-2-yl)benzamido)piperidine-1-carboxylate

Using essentially the same procedure described in Example 2 and employing N-Boc-3-aminopiperidine and 4-indazol-2-yl-benzoic acid as starting material, the tert-butyl 4-(4-(2H-indazol-2-yl)benzamido)piperidine-1-carboxylate was obtained as a pink solid, mp 202-204° C., MS (ES) m/z 421.3 [M+H]⁺;

### Step 1b: tert-butyl 4-(4-(1H-indazol-1-yl)benzamido)piperidine-1-carboxylate

Using essentially the same procedure described in Example 2 and employing N-Boc-3-aminopiperidine and 4-indazol-1-yl-benzoic acid as the starting material, tert-butyl 4-(4-(1H-indazol-1-yl)benzamido)piperidine-1-carboxylate was obtained as a yellow solid, mp 165-166° C., MS (ES) m/z 421.3 [M+H]⁺.

### Step 2a: tert-butyl 4-(4-(2H-indazol-2-yl)-N-methylbenzamido)piperidine-1-carboxylate

Using essentially the same procedure described in Example 3 (step 1) and employing tert-butyl 4-(4-(2H-indazol-2-yl)benzamido)piperidine-1-carboxylate as the starting material, the 4-[(4-indazol-2-yl-benzoyl)-methyl-amino]-piperidine-1-carboxylic acid tert-butyl ester was obtained as a yellow solid, mp 176-177° C., MS (ES) m/z 435.2 [M+H]⁺.

### Step 2b: tert-butyl 4-(4-(1H-indazol-1-yl)benzamido)piperidine-1-carboxylate

Using essentially the same procedure described in Example 3 (step 1) and employing and tert-butyl 4-(4-(1H-indazol-1-yl)benzamido)piperidine-1-carboxylate as the starting material, 4-[(4-indazol-1-yl-benzoyl)-methyl-amino]-piperidine-1-carboxylic acid tert-butyl ester was obtained as a yellow solid, mp 147-149° C., MS (ES) m/z 435.3 [M+H]⁺.

### Step 3a: tert-butyl 4-(4-(1H-indazol-1-yl)benzamido)piperidine-1-carboxylate

Using essentially the same procedure described in Example 3 (step 2) and employing tert-butyl 4-(4-(2H-indazol-2-yl)benzamido)piperidine-1-carboxylate as the starting material, the desired product 67 was obtained as a white solid, mp 260° C. decompose, MS (ES) m/z 335.1 [M+H]⁺.

### Step 3b: tert-butyl 4-(4-(1H-indazol-1-yl)benzamido)piperidine-1-carboxylate and tert-butyl 4-(4-(1H-indazol-1-yl)benzamido)piperidine-1-carboxylate

Using essentially the same procedure described in Example 3 (step 2) and employing tert-butyl 4-(4-(1H-indazol-1-yl)benzamido)piperidine-1-carboxylate as the starting material, the desired product 68 was obtained as a light yellow solid, mp 255-256° C., MS (ES) m/z 335.2 [M+H]⁺.

### Examples 69-74

### Preparation of 4-Indazol-2-yl-N-methyl-N-substituted piperidin-3-yl-benzamide hydrochloride compounds (69-71) and 4-Indazol-1-yl-N-methyl-N-substituted piperidin-3-yl-benzamide hydrochloride compounds (72-74)

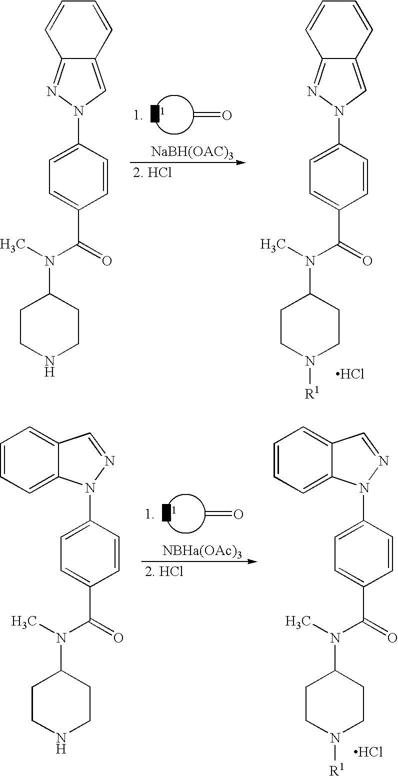

Using essentially the same procedure described in Example 5 and employing the desired ketone, the compounds shown in Table VIII were obtained and identified by NMR and mass spectral analyses.

TABLE VIII

| | |
|---|---|
| 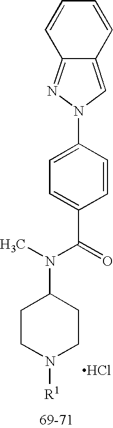 | 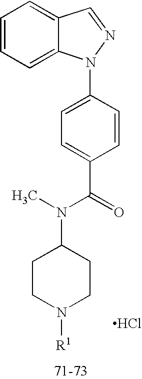 |

| Ex. No. | R¹ | mp ° C. | [M + H] |
|---|---|---|---|
| 69 | i-propyl | 166-168 | 377.2 |
| 70 | cyclobutyl | 273-275 | 389.2 |
| 71 | cyclopentyl | 283-285 | 403.2 |
| 72 | i-propyl | 151-162 | 377.2 |
| 73 | cyclobutyl | 160 (dec) | 389.2 |
| 74 | cyclopentyl | 240 (dec) | 403.2 |

### Example 75

### Preparation of (R)-4-((1H-indazol-1-yl)methyl)-N-methyl-N-(pyrrolidin-3yl)benzamide

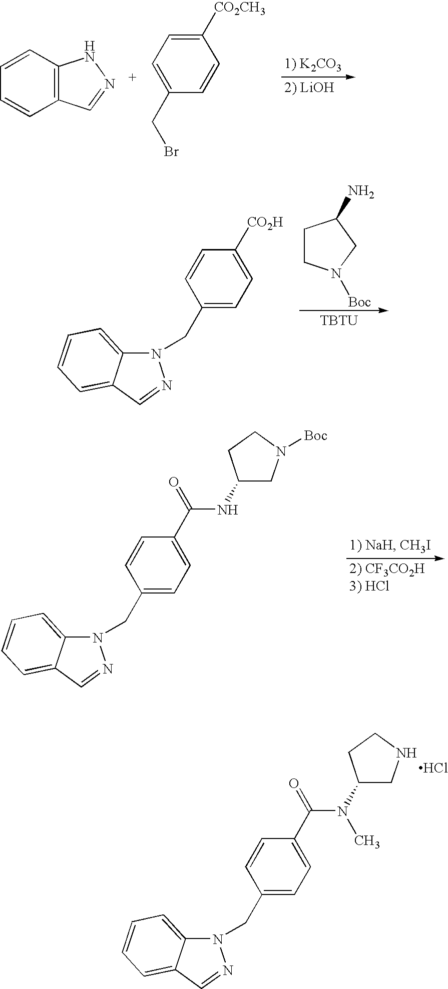

### Step 1: methyl 4-((1H-indazol-1-yl)methyl)benzoate

Using essentially the same procedure described in Example 1 (step 1) and employing indazole as the starting material, the title product was obtained as a white solid, mp 89-90° C., MS (ES) m/z 267.1 [M+H]⁺.

### Step 2: 4-((1H-indazol-1-yl)methyl)benzoic acid

Using essentially the same procedure described in Example 1 (step 2) and employing methyl 4-((1H-indazol-1-yl)methyl)benzoate as starting material, 4-indazol-1-ylmethyl-benzoic acid was obtained as a white solid, mp 178-179° C., MS (ES) m/z 253.1 [M+H]⁺.

### Step 3: (R)-tert-butyl 3-(4-((1H-indazol-1-yl)methyl)benzamido)pyrrolidine-1-carboxylate

Using essentially the same procedure described in Example 2 and employing 4-indazol-1-ylmethyl-benzoic acid as the starting material, 3-(4-indazol-1-ylmethyl-benzoylamino)-pyrrolidine-1-carboxylic acid tert-butyl ester was obtained as a yellow foam, [α]_{D}²⁵=−23.0° (c=1.00 in methanol), MS (ES) m/z 421.3 [M+H]⁺.

### Step 4: 3-[(4-Indazol-1-ylmethyl-benzoyl)-methyl-amino]-(R)-pyrrolidine-1-carboxylic acid tert-butyl ester

Using essentially the same procedure described in Example 3 (step 1) and employing 3-(4-indazol-1-ylmethyl-benzoylamino)-pyrrolidine-1-carboxylic acid tert-butyl ester as the starting material, the title product was obtained as a yellow wax, [α]_{D}²⁵=+55.0° (c=1.00 in methanol), MS (ES) m/z 435.3 [M+H]⁺.

### Step 5: 4-Indazol-1-ylmethyl-N-methyl-N-pyrrolidin-3-yl-benzamide hydrochloride

Using essentially the same procedure described in Example 3 (step 2) and employing 3-[(4-indazol-1-ylmethyl-benzoyl)-methyl-amino]-(R)-pyrrolidine-1-carboxylic acid tert-butyl ester as the starting material, the titled product was obtained as a yellow solid, mp 255-256° C. [α]_{D}²⁵=0° (c=1.00 in methanol), MS (ES) m/z 335.2 [M+H]⁺.

### Example 76-78

### Preparation of (R)-4-Indazol-1-ylmethyl-N-(1-substituted-pyrrolidin-3-yl)-N-methyl-benzamide hydrochloride compounds

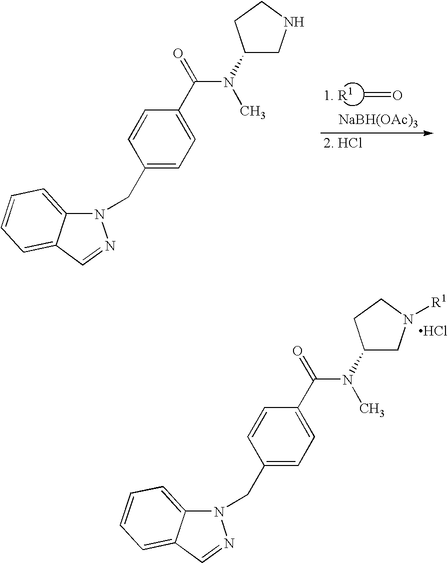

Using essentially the same procedure described in Example 5 and employing the desired ketone, the compounds shown in Table IX were obtained and identified by NMR and mass spectral analyses.

TABLE IX

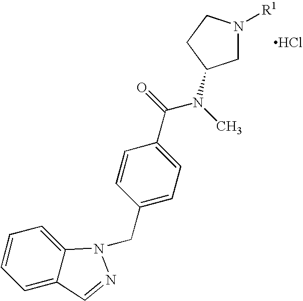

| Ex. No. | R¹ | mp ° C. | [M + H] | [α]_{D}^{25*} |
|---|---|---|---|---|
| 76 | i-propyl | 156-158 | 377.2 | +3 |
| 77 | cyclobutyl | 105-106 | 389.3 | +4 |
| 78 | cyclopentyl | 98-99 | 403.3 | — |

*1.00% solution in methanol

### Examples 79-81

### Preparation of 4-Indazol-1-ylmethyl-N-(1-substituted-piperidin-4-yl)-N-methyl-benzamide hydrochloride compounds

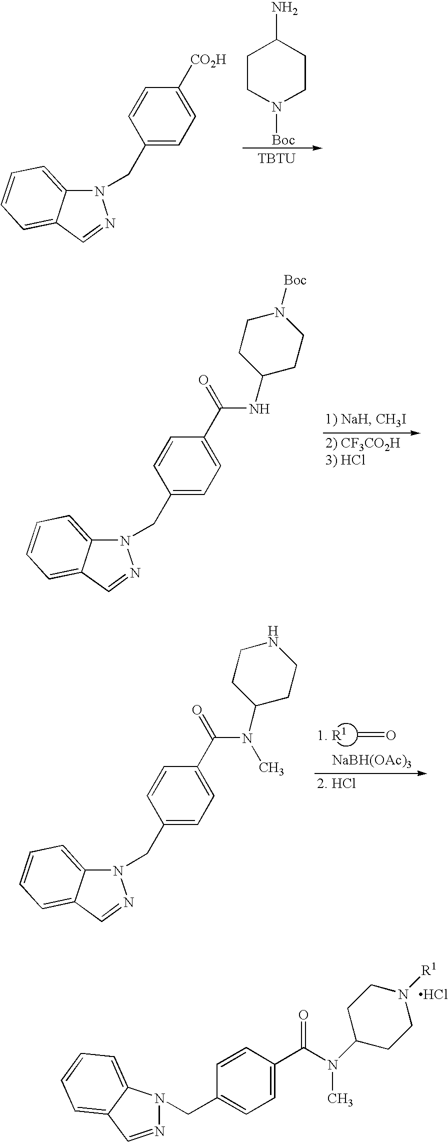

### Step 1: 3-(4-Indazol-1-ylmethyl-benzoylamino)-piperidine-1-carboxylic acid tert-butyl ester

Using essentially the same procedure described in Example 2 and employing 4-Indazol-1-ylmethyl-benzoic acid as the starting material, the title product was obtained as a white foam, MS (ES) m/z 435.2 [M+H]⁺.

### Step 2: 4-[(4-Indazol-1-ylmethyl-benzoyl)-methyl-amino]-piperidine-1-carboxylic acid tert-butyl ester

Using essentially the same procedure described in Example 3 (step 1) and employing 3-(4-indazol-1-ylmethyl-benzoylamino)-piperidine-1-carboxylic acid tert-butyl ester as the starting material, the title product was obtained as a yellow foam, MS (ES) m/z 449.2 [M+H]⁺.

### Step 3: 4-Indazol-1-ylmethyl-N-methyl-N-piperidin-4-yl-benzamide

Using essentially the same procedure described in Example 3 (step 2) and employing 4-[(4-indazol-1-ylmethyl-benzoyl)-methyl-amino]-piperidine-1-carboxylic acid tert-butyl ester as the starting material, the title product was obtained as a white solid, mp 146-148° C., MS (ES) m/z 349.2 [M+H]⁺.

### Step 4: 4-Indazol-1-ylmethyl-N-(1-substituted-piperidin-4-yl)-N-methyl-benzamide hydrochlorides

Using essentially the same procedure described in Example 5 and employing the desired ketone, the compounds shown in Table X were obtained and identified by NMR and mass spectral analyses.

TABLE X

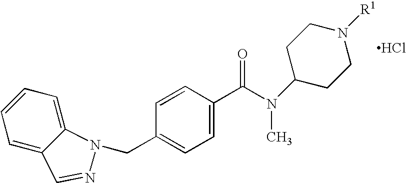

| Ex. No. | R¹ | mp ° C. | [M + H] |
|---|---|---|---|
| 79 | i-propyl | 197-199 | 391.2 |
| 80 | cyclobutyl | 254-256 | 403.2 |
| 81 | cyclopentyl | 265-267 | 417.2 |

### Examples 82-84

### Preparation of (R)—N-(1-substituted-pyrrolidin-3-yl)-N-methyl-4-pyrazol-1-yl-benzamide hydrochloride compounds

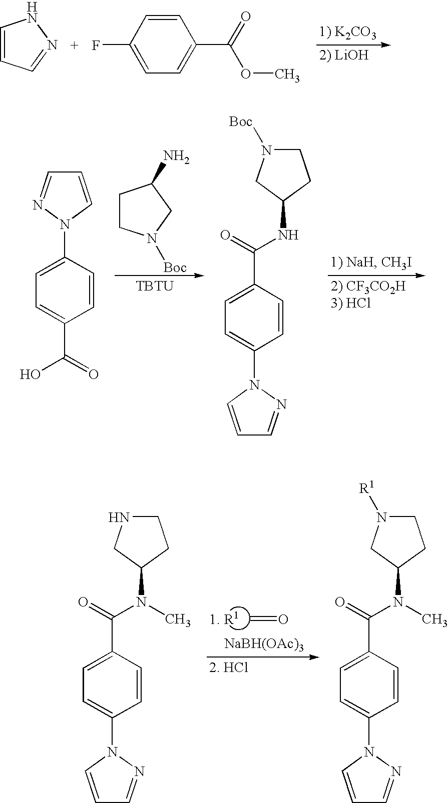

### Step 1: Methyl 4-(1H-pyrazol-1-yl)benzoate

Using essentially the same procedure described in Example 1 (step 1) and employing pyrazole as the starting material, methyl 4-(1H-pyrazol-1-yl)benzoate was obtained as a white solid, mp 107-109° C., MS (ES) m/z 203.2 [M−H]⁻

### Step 2: 4-(1H-pyrazol-1-yl)benzoic acid

Using essentially the same procedure described in Example 1 (step 2) and employing methyl 4-(1H-pyrazol-1-yl)benzoate as starting material, 4-pyrazol-1-yl-benzoic acid was obtained as a white solid, mp 263-264° C., MS (ES) m/z 187.0 [M−H]⁻.

### Step 3: (R)-tert-butyl 3-(4-(1H-pyrazol-1-yl)benzamido)pyrrolidine-1-carboxylate

Using essentially the same procedure described in Example 2 and employing 4-pyrazol-1-yl-benzoic acid as starting material, 3-(4-pyrazol-1-yl-benzoylamino)-pyrrolidine-1-carboxylic acid tert-butyl ester was obtained as an off-white solid, mp 263-264° C., [α]_{D}²⁵=−32.0° (c=1.00 in methanol), MS (ES) m/z 357.0 [M+H]⁺.

### Step 4: (R)-tert-butyl 3-(N-methyl-4-(1H-pyrazol-1-yl)benzamido)-pyrrolidine-1-carboxylate

Using essentially the same procedure described in Example 3 (step 1) and employing 3-(4-pyrazol-1-yl-benzoylamino)-pyrrolidine-1-carboxylic acid tert-butyl ester as the starting material, the title compound was obtained as an off-white foam, [α]_{D}²⁵=−7° (c=1.00 in methanol), MS (ES) m/z 393.2 [M+Na]⁺.

### Step 5: (R)—N-methyl-4-(1H-pyrazol-1-yl)-N-(pyrrolidin-3-yl)benzamide

Using essentially the same procedure described in Example 3 (step 2) and employing (R)-tert-butyl 3-(N-methyl-4-(1H-pyrazol-1-yl)benzamido)pyrrolidine-1-carboxylate as the starting material, the title compound was obtained as an off-white solid, mp 170-174° C., [α]_{D}²⁵=−9° (c=1.00 in methanol), MS (ESI) m/z 271.2 [M+H]⁺.

### Step 6. (R)—N-(1-substituted-pyrrolidin-3-yl)-N-methyl-4-pyrazol-1-yl-benzamide hydrochlorides

Using essentially the same procedure described in Example 5 and employing the desired ketone, the compounds shown in Table XI were obtained and identified by NMR and mass spectral analyses.

TABLE XI

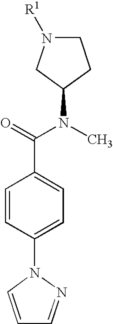

| Ex. No. | R¹ | mp ° C. | [M + H] | [α]_{D}^{25*} |
|---|---|---|---|---|
| 82 | i-propyl | 176-178 | 313.2 | +7.00 |
| 83 | cyclobutyl | 163-164 | 325.2 | +8.00 |
| 84 | cyclopentyl | 170-171 | 339.2 | +12.0 |

*1.00% solution in methanol

### Examples 85-87

### Preparation of N-methyl-N-(1-substituted piperidin-4-yl)-4-(1H-pyrazol-1-yl)benzamide hydrochloride compounds

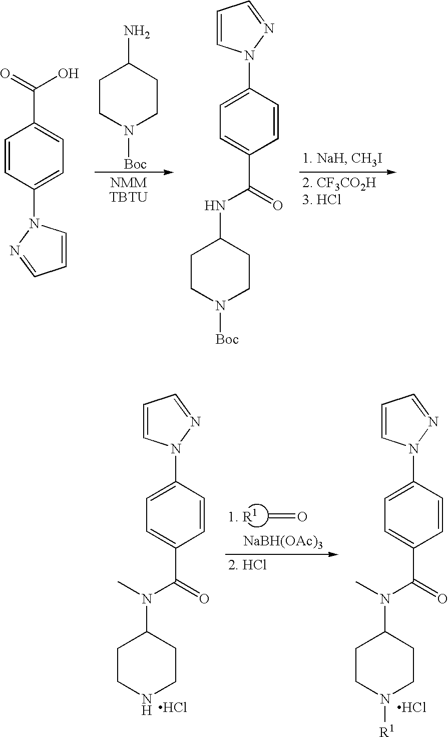

### Step 1: tert-butyl 4-(4-(1H-pyrazol-1-yl)benzamido)piperidine-1-carboxylate

Using essentially the same procedure described in Example 2 and employing 4-pyrazol-1-yl-benzoic acid as the starting material, the title compound was obtained as a white solid, mp 170-171° C., MS (ES) m/z 393.1 [M+Na]⁺.

### Step 2: tert-butyl 4-(N-methyl-4-(1H-pyrazol-1-yl)benzamido)piperidine-1-carboxylate

Using essentially the same procedure described in Example 3 (step 1) and employing tert-butyl 4-(4-(1H-pyrazol-1-yl)benzamido)piperidine-1-carboxylate as the starting material, the title compound was obtained as a white solid, mp 164-166° C., MS (ESI) m/z 407.2 [M+Na]⁺.

### Step 3: N-methyl-N-(piperidin-4-yl)-4-(1H-pyrazol-1-yl)benzamide

Using essentially the same procedure described in Example 3 (step 2) and employing tert-butyl 4-(N-methyl-4-(1H-pyrazol-1-yl)benzamido)piperidine-1-carboxylate as the starting material, N-methyl-N-(piperidin-4-yl)-4-(1H-pyrazol-1-yl)benzamide hydrochloride was obtained as an off-white solid, mp 162-163° C., MS (ESI) m/z 285.1 [M+H]⁺.

### Step 4: N-methyl-N-(1-substituted piperidin-4-yl)-4-(1H-pyrazol-1-yl)benzamide hydrochlorides

Using essentially the same procedure described in Example 5 and employing the desired ketone, the compounds shown in Table XII were obtained and identified by NMR and mass spectral analyses.

TABLE XII

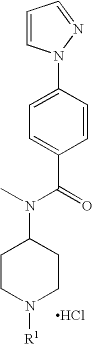

| Ex. No. | R¹ | mp ° C. | [M + H] |
|---|---|---|---|
| 85 | i-propyl | 285-286 | 327.2 |
| 86 | cyclobutyl | 272-273 | 339.2 |
| 87 | cyclopentyl | 240 (dec) | 353.2 |

### Examples 88-90

### Preparation of N-(1-substituted-pyrrolidin-3-yl)-N-methyl-4-pyrazol-1-ylmethyl-benzamide hydrochloride compounds

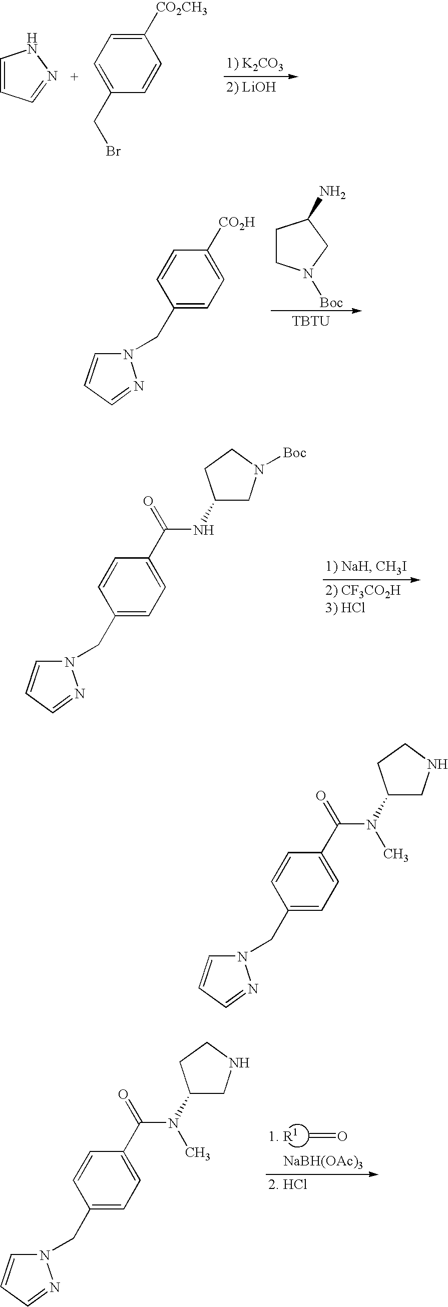

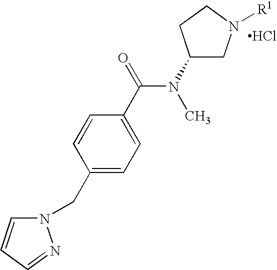

### Step 1: methyl 4-((1H-pyrazol-1-yl)methyl)benzoate

Using essentially the same procedure described in Example 1 (step 1) and employing pyrazole as the starting material, the title compound was obtained as a yellow oil, MS (ESI) m/z 217.1 [M+H]⁺.

### Step 2: 4-((1H-pyrazol-1-yl)methyl)benzoic acid

Using essentially the same procedure described in Example 1 and employing methyl 4-((1H-pyrazol-1-yl)methyl)benzoate as the starting material, 4-((1H-pyrazol-1-yl)methyl)benzoic acid was obtained as an off-white solid, mp 174-176° C., MS (ESI) m/z 203.0 [M+H]⁺.

### Step 3: (R)-tert-butyl 3-(4-((1H-pyrazol-1-yl)methyl)benzamido)pyrrolidine-1-carboxylate

Using essentially the same procedure described in Example 2 and employing 4-((1H-pyrazol-1-yl)methyl)benzoic acid as the starting material, 3-(4-pyrazol-1-ylmethyl-benzoylamino)-pyrrolidine-1-carboxylic acid tert-butyl ester was obtained as a white foam, [α]_{D}²⁵=0° (c=1.00 in methanol), MS (ESI) m/z 369.2 [M−H]⁻.

### Step 4: (R)-tert-butyl 3-(4-((1H-pyrazol-1-yl)methyl)-N-methylbenzamido)pyrrolidine-1-carboxylate

Using essentially the same procedure described in Example 3 (step 1) and employing (R)-tert-butyl 3-(4-((1H-pyrazol-1-yl)methyl)benzamido)pyrrolidine-1-carboxylate as the starting material, the title compound was obtained as a yellow foam, [α]_{D}²⁵=+111° (c=1.00 in methanol), MS (ESI) m/z 407.2 [M+Na]⁺.

### Step 5: (R)-4-((1H-pyrazol-1-yl)methyl)-N-methyl-N-(pyrrolidin-3-yl)benzamide

Using essentially the same procedure described in Example 3 (step 2) and employing (R)-tert-butyl 3-(4-((1H-pyrazol-1-yl)methyl)-N-methylbenzamido)pyrrolidine-1-carboxylate as the starting material, 4-pyrazol-1-ylmethyl-N-pyrrolidin-3-yl-benzamide hydrochloride was obtained as a light-yellow solid, mp 103-105° C., [α]_{D}²⁵=−2.0° (c=1.00 in methanol), MS (ESI) m/z 285.1 [M+H]⁺.

### Step 6: (R)-4-((1H-pyrazol-1-yl)methyl)-N-methyl-N-(1-substituted pyrrolidin-3-yl)benzamide hydrochlorides

Using essentially the same procedure described in Example 5 and employing the desired ketone, the compounds shown in Table XIII were obtained and identified by NMR and mass spectral analyses.

TABLE XIII

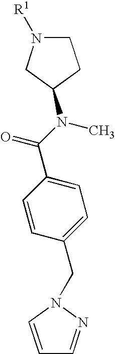

| Ex. No. | R¹ | mp ° C. | [M + H] | [α]_{D}^{25*} |
|---|---|---|---|---|
| 88 | i-propyl | 135-136 | 327.2 | −2 |
| 89 | cyclobutyl | 125-127 | 339.2 | +2 |
| 90 | cyclopentyl | 130-131 | 353.2 | +2 |

*1.00% solution in methanol

### Examples 91-93

### Preparation of N-(1-substituted-piperidin-4-yl)-N-methyl-4-pyrazol-1-ylmethyl-benzamide hydrochloride compounds

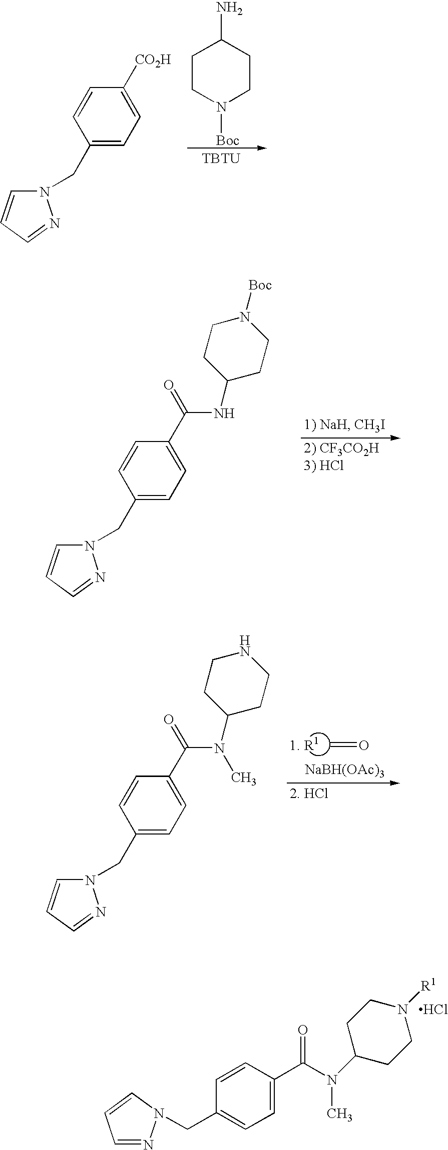

### Step 1: tert-butyl 4-(4-((1H-pyrazol-1-yl)methyl)benzamido)piperidine-1-carboxylate

Using essentially the same procedure described in Examples 2 and employing 4-pyrazol-1-ylmethyl-benzoic acid as the starting material, the title product was obtained as a white solid, mp 168-169° C., MS (ESI) m/z 383.2 [M−H]⁻.

### Step 2: tert-butyl 4-(4-((1H-pyrazol-1-yl)methyl)-N-methylbenzamido)piperidine-1-carboxylate

Using essentially the same procedure described in Example 3 (step 1) and employing 3-(4-pyrazol-1-ylmethyl-benzoylamino)-piperidine-1-carboxylic acid tert-butyl ester as the starting material, the title product which was obtained as a light yellow foam, MS (ESI) m/z 399.2 [M−H]⁻.

### Step 3: 4-((1H-pyrazol-1-yl)methyl)-N-methyl-N-(piperidin-4-yl)benzamide hydrochloride

Using essentially the same procedure described in Example 3 (step 2) and employing 4-[(4-indazol-1-ylmethyl-benzoyl)-methyl-amino]-piperidine-1-carboxylic acid tert-butyl ester as the starting material, the title product was obtained as an off-white solid, mp 110-112° C., MS (ESI) m/z 299.2 [M+H]⁺.

### Step 4: N-(1-substituted-piperidin-4-yl)-N-methyl-4-pyrazol-1-ylmethyl-benzamide hydrochlorides

Using essentially the same procedure described in Example 5 and employing the desired ketone, the compounds shown in Table XIV were obtained and identified by NMR and mass spectral analyses.

TABLE XIV

| Ex. No. | R¹ | mp ° C. | [M + H] |
|---|---|---|---|
| 91 | i-propyl | 213-215 | 341.2 |
| 92 | cyclobutyl | 235-237 | 353.2 |
| 93 | cyclopentyl | 250 (dec) | 367.2 |

### Examples 94

### Preparation of (1-Isopropyl-pyrrolidin-3-yl)-methyl-amine (94a) and 1-Cyclobutyl-pyrrolidin-3-yl)-methylamine (94b)

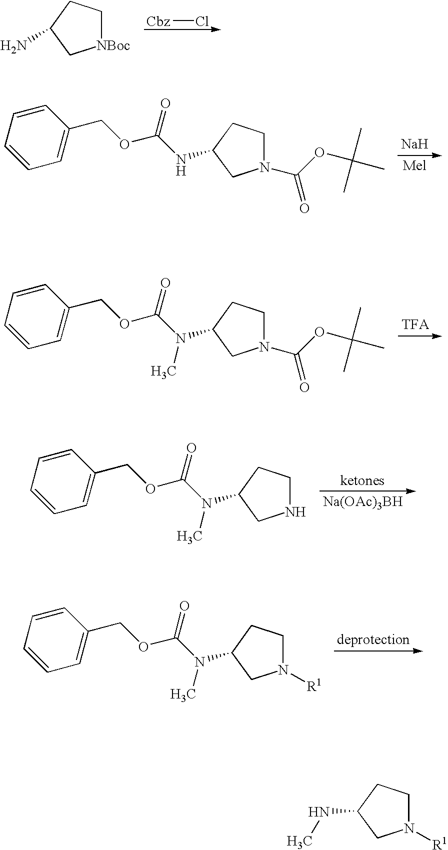

### Step 1: (R)-tert-Butyl 3-(benzyloxycarbonylamino)pyrrolidine-1-carboxylate

To a solution of (R)-tert-butyl 3-aminopyrrolidine-1-carboxylate (1.0 eq) in tetrahydrofuran at 0° C. was added benzyl chloroformate (1.2 eq) and diisopropylethylamine (2.5 eq) and the reaction mixture was stirred at room temperature for 2 hours. The reaction mixture was diluted with methylene chloride and washed with aqueous sodium hydroxide (1.0 N). The organic layer was dried (sodium sulfate) and the solvent was removed in vacuo. Purification by ISCO CombiFlash® chromatography (silica, 20-100% ethyl acetate in hexanes) provided the title compound, MS (ES) m/z 320.4 [M−H]⁻.

### Step 2: (R)-tert-Butyl 3-((benzyloxycarbonyl)(methyl)amino)-pyrrolidine-1-carboxylate

Using essentially the same procedure described in Example 3 (step 1) and employing (R)-tert-butyl 3-(benzyloxycarbonylamino)pyrrolidine-1-carboxylate as the starting material, the title compound was obtained as a colorless oil, MS (ES) m/z 334.4 [M+H]⁺.

### Step 3: (R)-Benzyl methyl(pyrrolidin-3-yl)carbamate

Using essentially the same procedure described in Example 3 (step 2) and employing (R)-tert-Butyl 3-((benzyloxycarbonyl)(methyl)amino)-pyrrolidine-1-carboxylate as the starting material, the title compound was obtained as a colorless oil, MS (ES) m/z 234.3 [M+H]⁺.

### Step 4a: (R)-Benzyl 1-isopropylpyrrolidin-3-yl(methyl)carbamate

Using essentially the same procedure described in Example 5 and employing (R)-benzyl methyl-(pyrrolidin-3-yl)carbamate and acetone as starting material, the desired product was obtained, MS (ES) m/z 276.4 [M+H]⁺.

### Step 4b: (R)-Benzyl 1-cyclobutylpyrrolidin-3-yl(methyl)carbamate

Using essentially the same procedure described in Example 5 and employing (R)-benzyl methyl-(pyrrolidin-3-yl)carbamate and cyclobutanone as starting materials, the desired product was obtained as an oil, MS (ES) m/z 288.4 [M+H]⁺.

### Step 5a: (1-Isopropyl-pyrrolidin-3-yl)-methyl-amine

To a solution of (R)-benzyl 1-isopropylpyrrolidin-3-yl(methyl)carbamate in ethanol at 0° C. under nitrogen atmosphere was added Pd—C 10% and the mixture was stirred at room temperature under hydrogen pressure (45 psi) overnight. The catalyst was removed by filtration and the solvent was concentrated in vacuo. The residue was purified by ISCO CombiFlash® chromatography (silica, 0-10% methanol in dichloromethane with 0.5% ammonium hydroxide) to afford (R)-1-isopropyl-N-methylpyrrolidin-3-amine, MS: (ESI) m/z 143.1 [M+H]⁺

### Step 5b: (1-Cyclobutyl-pyrrolidin-3-yl)-methyl-amine

Using essentially the same procedure described in Example 94 (5a) and employing (R)-Benzyl 1-cyclobutylpyrrolidin-3-yl(methyl)carbamate as the starting material, the desired product was obtained as a clear oil, MS: (ESI) m/z 155.1 [M+H]⁺

### Examples 95-102

### Preparation of substituted-4-(2-methyl-1H-benzo[d]imidazol-1-yl)benzoic acids

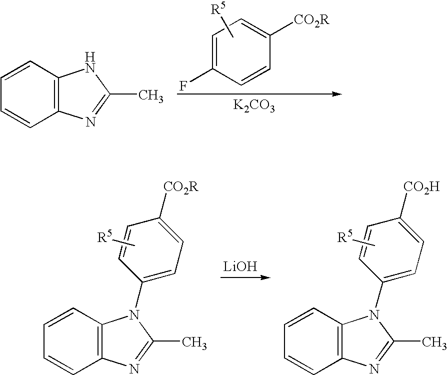

### Step 1: 4-(2-Methyl-benzoimidazol-1-yl)-substituted benzoates

Using essentially the same procedure described in Example 1 (step 1) and employing the desired methyl 4-fluorobenzoate as starting material, the desired products were obtained and identified by 1H NMR and mass spectral analyses.

TABLE XV

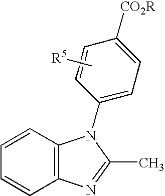

| Ex. No. | R | R⁵ | mp ° C. | [M + H] |
|---|---|---|---|---|
| 95a | Et | 3-fluoro | — | 299.1 |
| 96a | Me | 2-chloro | 148-150 | 301.1 |
| 97a | Me | 3-methyl | 135-136 | 281.1 |
| 98a | Me | 3-methoxyl | yellow oil | 297.1 |
| 99a | Me | 2-methoyl | colorless oil | 297.1 |
| 100a | Me | 3-CF₃ | yellow foam | 335.1 |
| 101a | Me | 2-CF₃ | 137-139 | 335.1 |
| 102a | Me | 2-Me | 134-135 | 281.1 |

### Step 2: 4-(2-Methyl-benzoimidazol-1-yl)-substituted benzoic acids

Using essentially the same procedure described in Example 1 (step 2) and employing the requisite 4-(2-methyl-benzoimidazol-1-yl)-substituted benzoate as starting material, the compounds shown in Table XVI were obtained and identified by NMR and mass spectral analyses.

TABLE XVI

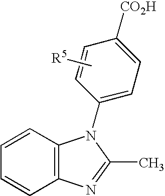

| Ex. No. | R⁵ | Appearance | mp ° C. | [M + H] |
|---|---|---|---|---|
| 95b | 3-fluoro | white solid | 285-287 | 271.0 |
| 96b | 2-chloro | white solid | 263-265 | 287.0 |
| 97b | 3-methyl | white solid | 250 decomp | 267.0 |
| 98b | 3-methoxyl | white solid | 254-256 | 283.1 |
| 99b | 2-methoxyl | white solid | 209-211 | 283.1 |
| 100a | 3-CF₃ | white solid | 292-294 | 321.2 |
| 101a | 2-CF₃ | white solid | 299-300 | 321.2 |
| 102a | 2-Me | white solid | 267-269 | 267.1 |

### Examples 103-118

### Preparation of (R)—N-methyl-4-(2-substituted-1H-benzo[d]imidazol-1-yl)-N-(1-methylpyrrolidin-3-yl)benzamideyl)benzamide hydrochloride compounds

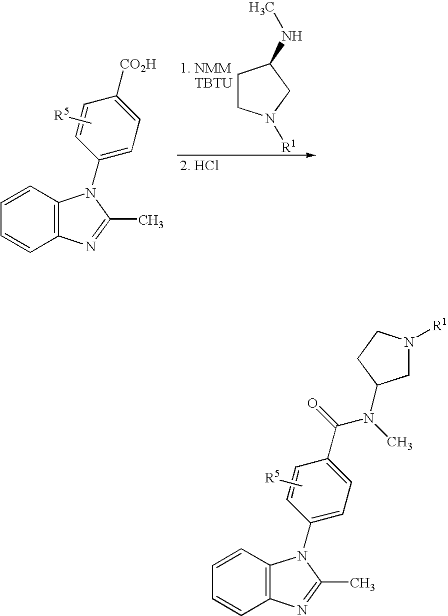

Using essentially the same procedure described in Example 2 and employing the desired methyl 4-(2-methyl-1h-benzo[d]imidazol-1-yl)benzoic acid and amine, the desired products were obtained and identified by 1H NMR and mass spectral analyses.

TABLE XVII

| Ex. No. | R¹ | R⁵ | mp ° C. | [M + H] | [α]_{D}²⁵* |
|---|---|---|---|---|---|
| 103 | i-propyl | 3-fluoro | 175-177 | 395.2 | — |
| 104 | cyclobutyl | 3-fluoro | 164-166 | 407.2 | — |
| 105 | i-propyl | 2-chloro | 204-205 | 411.2 | — |
| 106 | cyclobutyl | 2-chloro | 207-209 | 423.1 | — |
| 107 | i-propyl | 3-methyl | 183-185 | 391.2 | — |
| 108 | cyclobutyl | 3-methyl | 164-166 | 403.2 | +4 |
| 109 | i-propyl | 3-methoxyl | 168-170 | 407.2 | −5 |
| 110 | cyclobutyl | 3-methoxyl | 168-170 | 419.2 | −4 |
| 111 | i-propyl | 2-methoxyl | 193-195 | 407.2 | −8 |
| 112 | cyclobutyl | 2-methoxyl | 202-204 | 419.2 | −7 |
| 113 | i-propyl | 3-CF₃ | — | — | — |
| 114 | cyclobutyl | 3-CF₃ | — | — | — |
| 115 | i-propyl | 2-CF₃ | — | — | — |
| 116 | cyclobutyl | 2-CF₃ | — | — | — |
| 117 | i-propyl | 2-Me | — | — | — |
| 118 | cyclobutyl | 2-Me | — | — | — |

*1.00% solution in methanol

### Example 119-134

### Preparation of substituted-N-(1-substituted-piperidin-4-yl)-N-methyl-4-(2-methyl-benzoimidazol-1-yl)-benzamide hydrochloride compounds

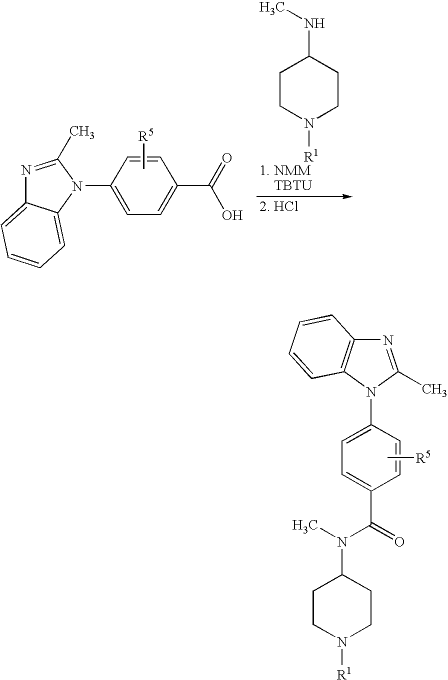

Using essentially the same procedure described in Example 2 employing the desired amine, the compounds shown in Table XVIII were obtained and identified by NMR and mass spectral analyses.

TABLE XVIII

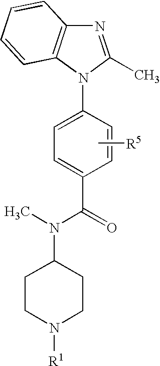

| Ex. No. | R¹ | R⁵ | mp ° C. | [M + H] |
|---|---|---|---|---|
| 119 | i-propyl | 3-fluoro | 279-281 | 409.2 |
| 120 | cyclopentyl | 3-fluoro | 250 (dec) | 435.2 |
| 121 | i-propyl | 2-chloro | 250 (dec) | 425.2 |
| 122 | cyclpentyl | 2-chloro | 240 (dec) | 451.2 |
| 123 | i-propyl | 3-methyl | 250 (dec) | 405.2 |
| 124 | cyclopentyl | 3-methyl | 240 (dec) | 431.2 |
| 125 | i-propyl | 3-methoxyl | 250-252 | 421.2 |
| 126 | cyclopentyl | 3-methoxyl | 244-246 | 447.2 |
| 127 | i-propyl | 2-methoxyl | 215-217 | 421.2 |
| 128 | cyclopentyl | 2-methoxyl | 210-212 | 447.2 |
| 129 | i-propyl | 3-CF₃ | — | — |
| 130 | cyclobutyl | 3-CF₃ | — | — |
| 131 | i-propyl | 2-CF₃ | — | — |
| 132 | cyclobutyl | 2-CF₃ | — | — |
| 133 | i-propyl | 2-Me | — | — |
| 134 | cyclobutyl | 2-Me | — | — |

### Example 135-137

### Preparation of N-(1-substituted-pyrrolidin-3-yl)-4-(2-methyl-benzoimidazol-1-yl)-benzamide hydrochloride compounds

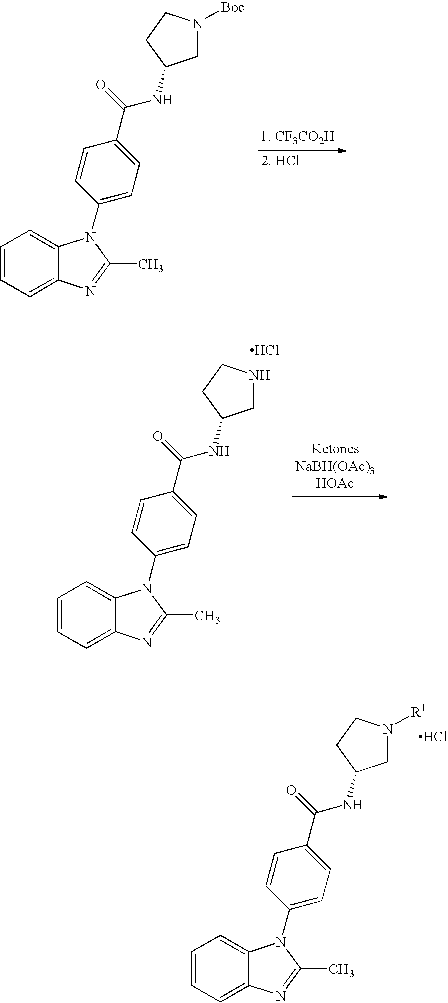

### Step 1: 4-(2-Methyl-benzoimidazol-1-yl)-N—(R)-pyrrolidin-3-yl-benzamide

Using essentially the same procedure described in Example 3 (step 2) and employing 3-[4-(2-methyl-benzoimidazol-1-yl)-benzoylamino]-(R)-pyrrolidine-1-carboxylic acid tert-butyl ester as the starting material, the title product was obtained as a yellow solid, mp 197-199° C.; [α]_{D}²⁵=0° (c=1.00 in methanol), MS (ES) m/z 321.2 [M+H]⁺.

### Step 2: N-(1-substituted-pyrrolidin-3-yl)-4-(2-methyl-benzoimidazol-1-yl)-benzamide hydrochlorides

Using essentially the same procedure described in Example 5 and employing 4-(2-methyl-benzoimidazol-1-yl)-N—(R)-pyrrolidin-3-yl-benzamide and the desired ketone as starting material, the compounds shown in Table XIX were obtained and identified by NMR and mass spectral analyses.

TABLE XIX

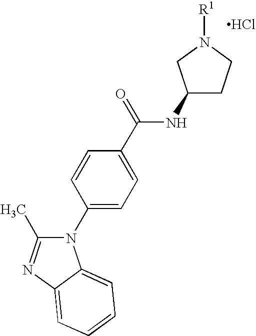

| Ex. No. | R¹ | mp ° C. | [M + H] | [α]_{D}^{25*} |
|---|---|---|---|---|
| 135 | isopropyl | 186-188 | 363.2 | −5.0 |
| 136 | cyclopentyl | 168-170 | 389.2 | −11.0 |
| 137 | cyclohexyl | 194-196 | 403.2 | −17.0 |

*1.00% solution in methanol

### Example 138

### Preparation of (R)—N-ethyl-4-(2-methyl-1H-benzo[d]imidazol-1-yl)-N-(pyrrolidin-3-yl)benzamide hydrochloride compounds

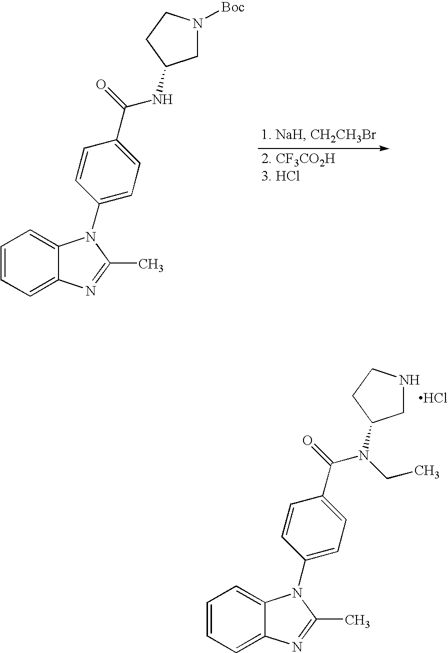

### Step 1: 3-{Ethyl-[4-(2-methyl-benzoimidazol-1-yl)-benzoyl]-amino}-(R)-pyrrolidine-1-carboxylic acid tert-butyl ester

Using essentially the same procedure described in Example 3 (step 1) and employing 3-[4-(2-methyl-benzoimidazol-1-yl)-benzoylamino]-(R)-pyrrolidine-1-carboxylic acid tert-butyl ester and ethyl bromide as the starting material, the title product was obtained as a white foam, [α]_{D}²⁵=+64.6° (1% solution in methanol); MS (ES) m/z 449.2 [M+H]⁺;

### Step 2: N-Ethyl-4-(2-methyl-benzoimidazol-1-yl)-N-pyrrolidin-3-yl-benzamide hydrochloride

Using essentially the same procedure described in Example 3 (step 2) and employing 3-{ethyl-[4-(2-methyl-benzoimidazol-1-yl)-benzoyl]-amino}-(R)-pyrrolidine-1-carboxylic acid tert-butyl ester as the starting material, the title product was obtained as a yellow solid, mp 174-176° C.; [α]_{D}²⁵=−11.4° (1% solution in methanol); MS (ES) m/z 349.2 [M+H]⁺;

### Example 139-141

### Preparation of (R)—N-ethyl-4-(2-methyl-1H-benzo[d]imidazol-1-yl)-N-(1-methylpyrrolidin-3-yl)benzamide hydrochloride compounds

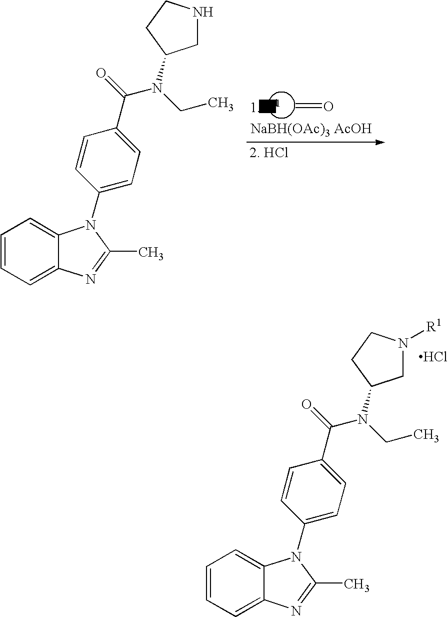

Using essentially the same procedure described in Example 5 and employing the appropriate ketone, the compounds shown in Table XXI were obtained and identified by NMR and mass spectral analyses.

TABLE XXI

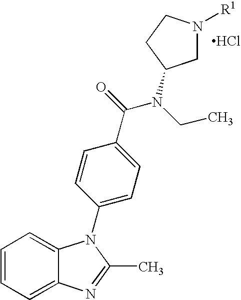

| Ex. No. | R¹ | mp ° C. | [M + H] | [α]_{D}^{25*} |
|---|---|---|---|---|
| 139 | isopropyl | 156-157 | 391.3 | −10.4 |
| 140 | cyclobutyl | 134-136 | 403.28 | −8.4 |
| 141 | cyclopentyl | 162-164 | 417.30 | −7.6 |
| 142 | cyclohexyl | 177-179 | 431.32 | −8.2 |

[α]_{D}^{25*} = 1% solution in methanol

### Example 143-144

### Preparation of (R)—N-methyl-4-((2-methyl-1H-benzo[d]imidazol-1-yl)methyl)-N-(1-substituted pyrrolidin-3-yl)-1-naphthamide hydrochloride compounds and N-methyl-4-((2-methyl-1H-benzo[d]imidazol-1-yl)methyl)-N-(1-substituted piperidin-4-yl)-1-naphthamide hydrochloride compounds

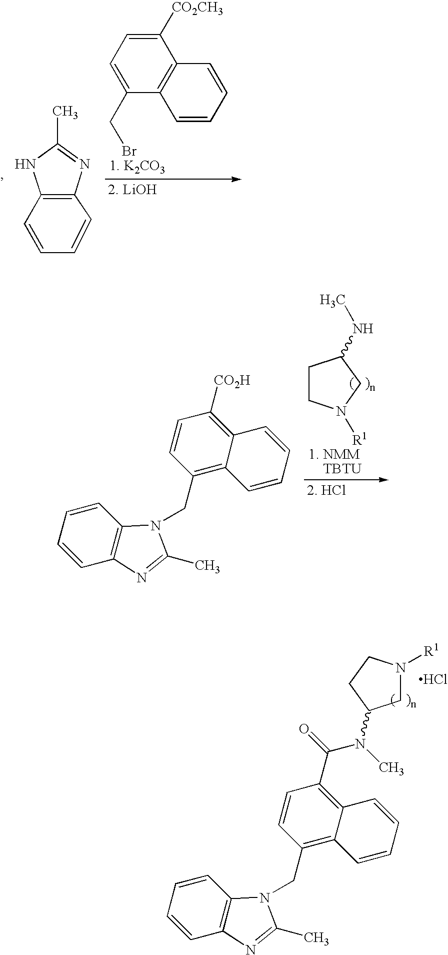

### Step 1: methyl 4-((2-methyl-1H-benzo[d]imidazol-1-yl)methyl)-1-naphthoate

Using essentially the same procedure described in Example 3 (step 1) and employing methyl 4-(bromomethyl)-1-naphthoate as the starting material, the title product was obtained as a white solid, mp 207-208° C., MS (ES) m/z 331.1 [M+H]⁺;

### Step 2: 4-((2-methyl-1H-benzo[d]imidazol-1-yl)methyl)-1-naphthoic acid

Using essentially the same procedure described in Example 3 (step 2) and employing methyl 4-((2-methyl-1H-benzo[d]imidazol-1-yl)methyl)-1-naphthoate as the starting material, the title product was obtained as a white solid, mp 292-293° C., MS (ES) m/z 317.1 [M+H]⁺;

### Step 3: (R)—N-methyl-4-((2-substituted-1H-benzo[d]imidazol-1-yl)methyl)-N-(1-methylpyrrolidin-3-yl)-1-naphthamide hydrochlorides and N-(1-substitutediperidin-4-yl)-N-methyl-4-((2-methyl-1H-benzo[d]imidazol-1-yl)methyl)-1-naphthamide hydrochlorides

Using essentially the same procedure described in Example 5 and employing the desired ketone, the compounds shown in Table XXII were obtained and identified by NMR and mass spectral analyses.

TABLE XXII

| Ex. No | n | R¹ | [M + H] | [α]_{D}^{25*} | mp ° C. |
|---|---|---|---|---|---|
| 143a | 1 | isopropyl | 441.2 | 0 | 192-194 |
| 143b | 1 | cyclobutyl | 453.2 | +3 | 157-159 |
| 144a | 2 | cyclopentyl | 481.2 | — | 228-230 |
| 144b | 2 | isopropyl | 455.2 | — | 153-155 |
| 144c | 2 | cyclobutyl | 467.2 | | 279-281 |

### Example 145-148

### Preparation of (R)—N-methyl-3-(2-methyl-1H-benzo[d]imidazol-1-yl)-N-(1-substituted pyrrolidin-3-yl)benzamide hydrochloride compounds (145-146) and N-methyl-3-(2-methyl-1H-benzo[d]imidazol-1-yl)-N-(1-substituted piperidin-4-yl)benzamide hydrochloride compounds (147-148)

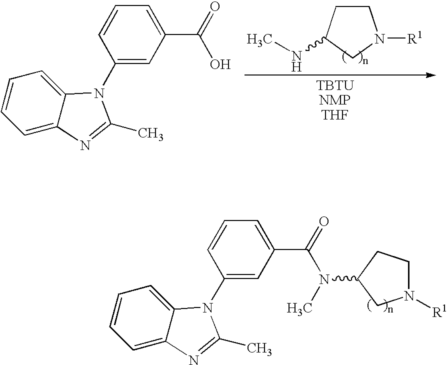

Using essentially the same procedure described in Example 2 employing the desired amine, the compounds shown in Table XXIII are obtained.

TABLE XXIII

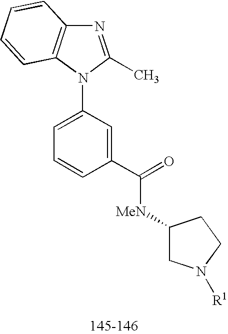

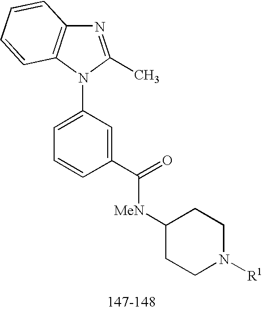

| Ex. No. | R¹ |
|---|---|
| 145 | i-propyl |
| 146 | cyclobutyl |
| 147 | i-propyl |
| 148 | cyclopenty |

### Example 149-180

### Preparation of (R)—N-methyl-3-(fluoro substituted 1H-benzo[d]imidazol-1-yl)-N-(1-substituted pyrrolidin-3-yl)benzamide hydrochloride compounds and N-methyl-3-(fluoro substituted-1H-benzo[d]imidazol-1-yl)-N-(1-substituted piperidin-4-yl)benzamide hydrochloride compounds

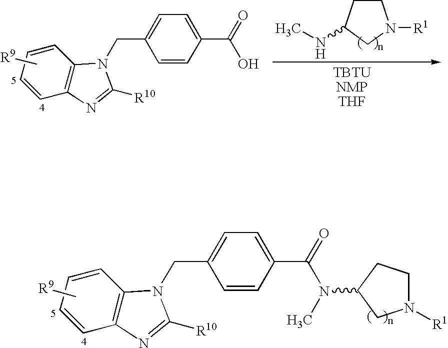

Using essentially the same procedure described in Example 2 and employing the desired 4-((fluoro substituted-1H-benzo[d]imidazol-1-yl)methyl)benzoic acid and amine, the compounds shown in Table XXIV are obtained.

TABLE XXIV

| Ex. | | | | | Ex. | | | | |
|---|---|---|---|---|---|---|---|---|---|
| No. | R⁹ | R¹⁰ | n | R¹ | No. | R⁹ | R¹⁰ | n | R¹ |
| 149 | 4-F | H | 1 | i-propyl | 150 | 4-F | H | 1 | cyclobutyl |
| 151 | 5-F | H | 1 | i-propyl | 152 | 5-F | H | 1 | cyclobutyl |
| 153 | 6-F | H | 1 | i-propyl | 154 | 6-F | H | 1 | cyclobutyl |
| 155 | 7-F | H | 1 | i-propyl | 156 | 7-F | H | 1 | cyclobutyl |
| 157 | 4-F | H | 2 | i-propyl | 158 | 4-F | H | 2 | cyclopentyl |
| 159 | 5-F | H | 2 | i-propyl | 160 | 5-F | H | 2 | cyclopentyl |
| 161 | 6-F | H | 2 | i-propyl | 162 | 6-F | H | 2 | cyclopentyl |
| 163 | 7-F | H | 2 | i-propyl | 164 | 7-F | H | 2 | cyclopentyl |
| 165 | 4-F | Me | 1 | i-propyl | 166 | 4-F | Me | 1 | cyclobutyl |
| 167 | 5-F | Me | 1 | i-propyl | 168 | 5-F | Me | 1 | cyclobutyl |
| 169 | 6-F | Me | 1 | i-propyl | 170 | 6-F | Me | 1 | cyclobutyl |
| 171 | 7-F | Me | 1 | i-propyl | 172 | 7-F | Me | 1 | cyclobutyl |
| 173 | 4-F | Me | 2 | i-propyl | 174 | 4-F | Me | 2 | cyclopentyl |
| 175 | 5-F | Me | 2 | i-propyl | 176 | 5-F | Me | 2 | cyclopentyl |
| 177 | 6-F | Me | 2 | i-propyl | 178 | 6-F | Me | 2 | cyclopentyl |
| 179 | 7-F | Me | 2 | i-propyl | 180 | 7-F | Me | 2 | cyclopentyl |

### Example 181-212

### Preparation of (R)—N-methyl-4-((1H-benzo[d]imidazol-1-yl)methyl)-N-(1-substituted pyrrolidin-3-yl)benzamide hydrochloride compounds and N-methyl-4-((2-methyl-1H-benzo[d]imidazol-1-yl)methyl)-N-(1-substituted piperidin-4-yl)benzamide hydrochloride compounds

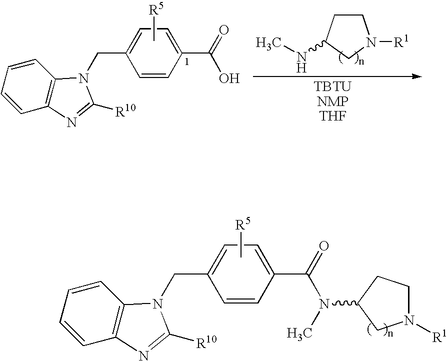

Using essentially the same procedure described in Example 2 and employing the desired 4-((1H-benzo[d]imidazol-1-yl)methyl)benzoic acid and amine, the compounds shown in Table XXV are obtained.

TABLE XXV

| Ex. | | | | | Ex. | | | | |
|---|---|---|---|---|---|---|---|---|---|
| No. | R⁵ | R¹⁰ | n | R¹ | No. | R⁵ | R¹⁰ | n | R¹ |
| 181 | 2-F | H | 1 | i-propyl | 182 | 2-F | H | 1 | cyclobutyl |
| 183 | 3-F | H | 1 | i-propyl | 184 | 3-F | H | 1 | cyclobutyl |
| 185 | 2-OMe | H | 1 | i-propyl | 187 | 2-OMe | H | 1 | cyclobutyl |
| 188 | 3-Cl | H | 1 | i-propyl | 189 | 3-Cl | H | 1 | cyclobutyl |
| 189 | 2-F | H | 2 | i-propyl | 190 | 2-F | H | 2 | cyclopentyl |
| 191 | 3-F | H | 2 | i-propyl | 192 | 3-F | H | 2 | cyclopentyl |
| 193 | 2-OMe | H | 2 | i-propyl | 194 | 2-OMe | H | 2 | cyclopentyl |
| 195 | 3-Cl | H | 2 | i-propyl | 196 | 3-Cl | H | 2 | cyclopentyl |
| 197 | 2-F | Me | 1 | i-propyl | 198 | 2-F | Me | 1 | cyclobutyl |
| 199 | 3-F | Me | 1 | i-propyl | 200 | 3-F | Me | 1 | cyclobutyl |
| 201 | 2-OMe | Me | 1 | i-propyl | 202 | 2-OMe | Me | 1 | cyclobutyl |
| 203 | 3-Cl | Me | 1 | i-propyl | 204 | 3-Cl | Me | 1 | cyclobutyl |
| 205 | 2-F | Me | 2 | i-propyl | 206 | 2-F | Me | 2 | cyclopentyl |
| 207 | 3-F | Me | 2 | i-propyl | 208 | 3-F | Me | 2 | cyclopentyl |
| 209 | 2-OMe | Me | 2 | i-propyl | 210 | 2-OMe | Me | 2 | cyclopentyl |
| 211 | 3-Cl | Me | 2 | i-propyl | 212 | 3-Cl | Me | 2 | cyclopentyl |

### Example 213-216

### Preparation of (R)-4-(1H-benzo[d]imidazol-1-yl)-N-methyl-N-(piperidin-3-yl)benzamide, (R)—N-methyl-4-(2-methyl-1H-benzo[d]imidazol-1-yl)-N-(piperidin-3-yl)benzamide and (R)-4-((1H-benzo[d]imidazol-1-yl)methyl)-N-methyl-N-(piperidin-3-yl)benzamide, (R)—N-methyl-4-((2-methyl-1H-benzo[d]imidazol-1-yl)methyl)-N-(piperidin-3-yl)benzamide

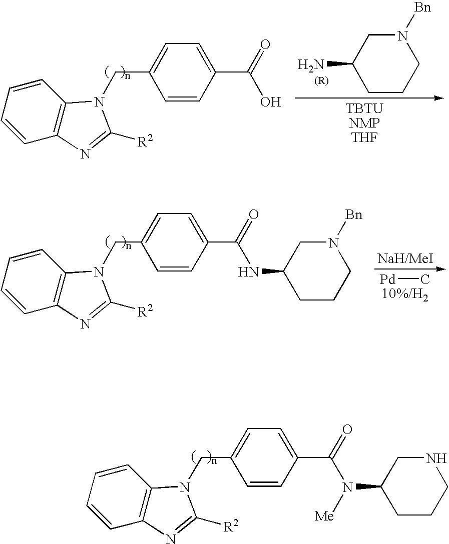

Step 1: Using essentially the same procedure described in Example 2 employing the desired 4-((1H-benzo[d]imidazol-1-yl)methyl)benzoic acid and (R)-1-benzylpiperidin-3-amine, the compounds shown in Table XXVI are obtained.

TABLE XXVI

| Ex. No. | R² | n | Ex. No. | R² | n |
|---|---|---|---|---|---|
| 213a | H | 0 | 214a | Me | 0 |
| 215a | H | 1 | 216a | Me | 1 |

Step 2: Using essentially the same procedure described in Example 3 (step 1), the compounds shown in Table XXVII are obtained.

TABLE XXVII

| Ex. No. | R² | n | Ex. No. | R² | n |
|---|---|---|---|---|---|
| 213b | H | 0 | 214b | Me | 0 |
| 215b | H | 1 | 216b | Me | 1 |

Step 3: To a solution of the desired substrate in ethanol under N2 at room temperature is added Pd—C 10%. The reaction mixture is hydrogenated at 40 Psi for 18 hrs. The mixture is filtered through a pad of celite and the filtrate is concentrated under in vacuo. The residue are purified by ISCO CombiFlash chromatography (silica, 2.5-3.5% methanol/methylene chloride) to provide the compounds shown in Table XXVII.

TABLE XXVIII

| Ex. No. | R² | n | Ex. No. | R² | n |
|---|---|---|---|---|---|
| 214c | H | 0 | 215c | Me | 0 |
| 216c | H | 1 | 217c | Me | 1 |

### Example 218-229

### Preparation of (R)-1-substituted-N-methylpiperidin-3-amine derivatives

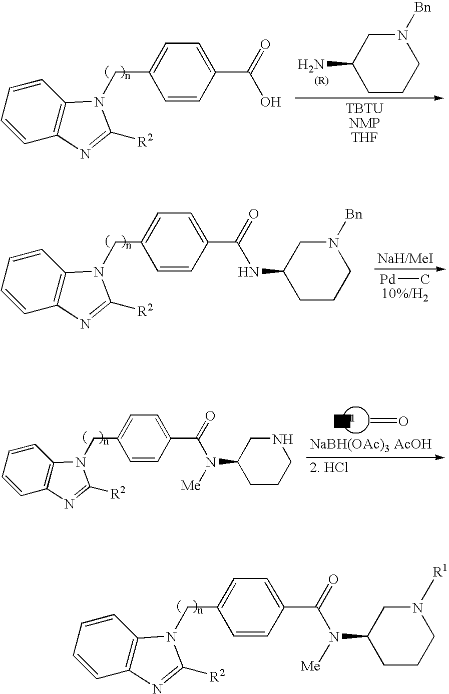

Using essentially the same procedure described in Example 2 employing the desired amine, the compounds shown in Table XXIX are obtained.

TABLE XXIX

| Ex. No. | R² | n | R¹ | Ex. No. | R² | n | R¹ |
|---|---|---|---|---|---|---|---|
| 218 | H | 0 | i-propyl | 219 | Me | 0 | i-propyl |
| 220 | H | 0 | cyclobutyl | 221 | Me | 0 | cyclobutyl |
| 222 | H | 0 | cyclopentyl | 223 | Me | 0 | cyclopentyl |
| 224 | H | 1 | i-propyl | 225 | Me | 1 | i-propyl |
| 226 | H | 1 | cyclobutyl | 227 | Me | 1 | cyclobutyl |
| 228 | H | 1 | cyclopentyl | 229 | Me | 1 | cyclopentyl |

### Example 229-240

### Preparation of (R)—N-(1-substituted pyrrolidin-3-yl)-N-methyl-4-((substituted-1H-benzo[d]imidazol-1-yl)methyl)benzamide hydrochloride compounds and N-(1-substituted piperidin-4-yl)-N-methyl-4-((substituted-1H-benzo[d]imidazol-1-yl)methyl)benzamide hydrochloride compounds

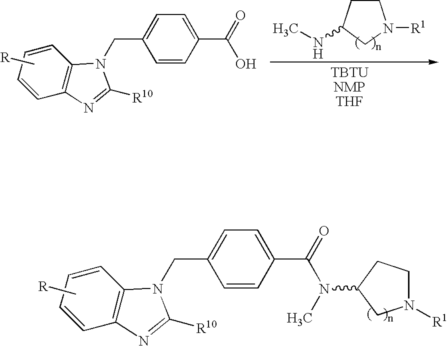

Using essentially the same procedures described in Example 2 employing the desired 4-((1H-benzo[d]imidazol-1-yl)methyl)benzoic acids and amines, the compounds shown in Table XXX are obtained.

TABLE XXX

| Ex. | | | | | | | |
|---|---|---|---|---|---|---|---|
| No. | R | n | R¹ | Ex. No. | R² | n | R¹ |
| 229 | 5-OMe | 0 | i-propyl | 230 | 6-OMe | 0 | i-propyl |
| 231 | 5-OMe | 0 | cyclobutyl | 232 | 6-OMe | 0 | cyclobutyl |
| 223 | 5-Me | 0 | i-propyl | 234 | 6-Me | 0 | i-propyl |
| 225 | 5-Me | 0 | cyclobutyl | 236 | 6-Me | 0 | cyclobutyl |
| 227 | 5-OMe | 1 | i-propyl | 238 | 6-OMe | 1 | i-propyl |
| 229 | 5-Me | 1 | cyclopentyl | 240 | 6-Me | 1 | cyclopentyl |

### Example 241-252

### Preparation of (R)—N-(1-substituted pyrrolidin-3-yl)-4-((substituted-2-methyl-1H-benzo[d]imidazol-1-yl)methyl)-N-methylbenzamidehydrochloride compounds and N-(1-substituted piperidin-4-yl)-4-((substituted-2-methyl-1H-benzo[d]imidazol-1-yl)methyl)-N-methylbenzamidehydrochloride compounds

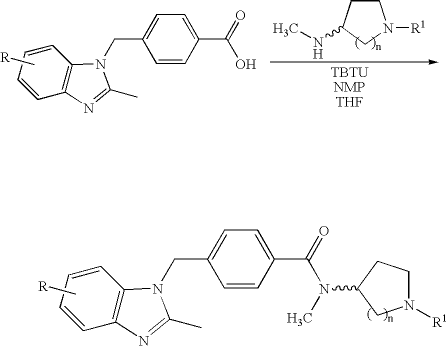

Using essentially the same procedures described in Examples 2 employing the desired 4-((1H-benzo[d]imidazol-1-yl)methyl)benzoic acids and amines, the compounds shown in Table XXXI are obtained.

TABLE XXXI

| Ex. | | | | | | | |
|---|---|---|---|---|---|---|---|
| No. | R | n | R¹ | Ex. No. | R² | n | R¹ |
| 241 | 5-OMe | 0 | i-propyl | 242 | 6-OMe | 0 | i-propyl |
| 243 | 5-OMe | 0 | cyclobutyl | 244 | 6-OMe | 0 | cyclobutyl |
| 245 | 5-Me | 0 | i-propyl | 246 | 6-Me | 0 | i-propyl |
| 247 | 5-Me | 0 | cyclobutyl | 248 | 6-Me | 0 | cyclobutyl |
| 249 | 5-OMe | 1 | i-propyl | 250 | 6-OMe | 1 | i-propyl |
| 251 | 5-Me | 1 | cyclopentyl | 252 | 6-Me | 1 | cyclopentyl |

### Example 253

### Evaluation of Methyl histamine binding in Human Histamine H3 Receptor Cell Line

The affinity of test compounds for the histamine 3 (H3) receptor is evaluated in the following manner. Stably transfected HEK293T cells are grown in DMEM containing 10% heat inactivated FBS and G-418 (500 ug/ml). Cells are scraped from the plate, transferred to centrifuge tubes, washed one time in PBS by centrifugation in a Sorvall RT7 Plus centrifuge (2000 rpm 10 minutes, 4° C.). The resulting pellets are stored at −80° C. until ready for use. Cells are re-suspended in buffer (50 mM Tris pH=7.5) and placed in a Dounce homogenizer, douncing ten times to homogenize cells. The homogenate is spun down by centrifugation (Sorvall RT7 Plus, 1800 rpm 10 minutes, 4° C.). The supernatant is placed in a Corex tube and spun down by centrifugation (Sorvall RC 5c Plus, 17,000 rpm 20 minutes, 4° C.). The pellet is resuspended in buffer (50 mM Tris, pH 7.5). Protein concentration (ug/ul) is determined using the Micro-BCA Protein Determination. The binding assay is set up in a 96 well microtiter plate in a total volume of 250 uL. Non-specific binding is determined in the presence of 10 uM clobenpropit. The final radioligand concentration is 1 nM. The test compound is serially diluted using the Beckman Biomek2000 to a final approximate range of 100 uM to 100 pM. Membranes are suspended in buffer, homogenized in 2 bursts of ten seconds using a Vitris mechanical homogenizer set at power setting 5. Ten μg of membranes are added to each well. Following a one hour incubation at 30° C., the reaction is terminated by the addition of ice cold buffer and rapid filtration with a Packard Filtermate Harvester through a GF/B filter pre-soaked with 1% PEI for one hour. The plate is dried for one hour at 37° C. and 60 μL Microscint Scintillant is added to each well. The CPM per well is measured on a Packard Top Count NXT. Ki values are determined in nM. The Ki is calculated from the IC₅₀ (i.e. the concentration of competing ligand which displaces 50% of the specific binding of the radioligand). CPM values are expressed as % specific binding and plotted vs compound concentration. A curve is fitted using a four-parameter logistic fit and the IC₅₀ value is determined. The Ki is calculated from this using the Cheng-Prusoff equation: pKi=IC₅₀/1+(L/Kd) where L=concentration of free radioligand used in the assay, and Kd is the dissociation constant of the radioligand for the receptor. L is determined for each experiment by counting an aliquot of the diluted radioligand (corresponding to that added to each well) and the Kd has previously been determined under identical conditions for this cell line/radioligand.

### Cyclic AMP Assay for Histamine Receptor H3 Antagonism Activity.

Stable H3 cells are maintained in tissue culture flask in DMEM with high glucose, 10% FBS, 1× pen/strep, 500 ug/ml GY18, until experiment. Culture media is removed and cells are washed twice with PBS w/Ca++ and Mg++ plus 500 μM IBMX. Cells are then detached by tapping on the side of the flask and resuspend in the same buffer. Two thousand cells/well are incubated with 1 μM histamine plus 10 μM forskolin plus various concentrations of compounds in a total volume of 30 μL in 96 well plates for 30 min at 30° C. Final test compound concentrations range from 10-4M to 10-9.5M at full log dilutions. Cyclic AMP levels are measured using HitHunter cAMP kit from Discoverx, cat# 900041 according to manufacturer's instruction. Chemiluminescence signals are detected using Top Count (Packard).

Cyclic AMP levels in control cells receiving 10 μM forskolin plus 100 nM histamine are considered 0%, and in cells receiving 10 uM forskolin plus 100 nM histamine plus 1 μM clobenpropit are considered 100%. Data are expressed as % control and analyzed using Prizm soft ware. The Kb values are calculated using the following equation, KB=EC₅₀ or IC₅₀/[1+ (ligand/Kd)]. The data are shown in Table XXX, below.

TABLE XXX

| | hH3 Binding Ki |
|---|---|
| Example # | (nM) |
| 3 | D |
| 4 | B |
| 5 | A |
| 6 | B |
| 7 | B |
| 8 | B |
| 9 | B |
| 10 | D |
| 11 | D |
| 12 | A |
| 13 | A |
| 14 | A |
| 15 | B |
| 16 | B |
| 17 | A |
| 18 | D |
| 19 | D |
| 20 | B |
| 21 | B |
| 22 | A |
| 23 | B |
| 24 | B |
| 25 | B |
| 26 | D |
| 27 | D |
| 28 | B |
| 29 | B |
| 30 | D |
| 31 | A |
| 32 | A |
| 33 | D |
| 34 | B |
| 35 | D |
| 36 | D |
| 37 | A |
| 38 | A |
| 39 | A |
| 40 | B |
| 41 | A |
| 42 | A |
| 43 | B |
| 44 | D |
| 45 | D |
| 46 | C |
| 47 | D |
| 48 | D |
| 49 | C |
| 50 | D |
| 51 | D |
| 53 | D |
| 54 | A |
| 55 | A |
| 56 | B |
| 60a | D |
| 60b | D |
| 61 | A |
| 62 | A |
| 63 | A |
| 64 | A |
| 65 | A |
| 66 | B |
| 67 | D |
| 68 | D |
| 69 | C |
| 70 | D |
| 71 | D |
| 72 | C |
| 73 | D |
| 74 | D |
| 75 | D |
| 76 | C |
| 77 | B |
| 78 | C |
| 79 | C |
| 80 | D |
| 81 | D |
| 82 | A |
| 83 | A |
| 84 | A |
| 85 | D |
| 86 | D |
| 87 | D |
| 88 | D |
| 89 | D |
| 90 | D |
| 91 | D |
| 92 | D |
| 93 | D |
| 103 | B |
| 104 | B |
| 105 | B |
| 106 | B |
| 107 | — |
| 108 | — |
| 109 | — |
| 110 | — |
| 111 | — |
| 112 | — |
| 113 | — |
| 114 | — |
| 115 | — |
| 116 | — |
| 117 | — |
| 118 | — |
| 119 | A |
| 120 | B |
| 121 | A |
| 122 | C |
| 123 | — |
| 124 | — |
| 125 | — |
| 126 | — |
| 127 | — |
| 128 | — |
| 129 | — |
| 130 | — |
| 131 | — |
| 132 | — |
| 133 | — |
| 134 | — |
| 135 | D |
| 136 | D |
| 137 | D |
| 138 | D |
| 139 | B |
| 140 | B |
| 141 | C |
| 142 | D |

For Table XXX
A = ≦10 nM
B = 10.1 nM-50.0 nM
C = 50.1 nM-100 nM
D = >100 nM

What is claimed is:

1. A compound of formula I 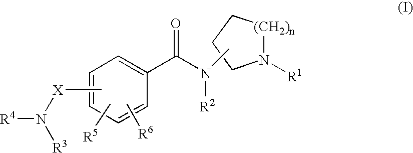
wherein
X is (CR⁷R⁸)ₘ, CO or SO₂;
m is 0 or 1;
n is 1, 2 or 3;
R¹ is H, C₁-C₆ alkyl, C₁-C₆ haloalkyl, C₃-C₁₀ cycloalkyl or a 3-10 membered cycloheteroalkyl each group optionally substituted;
R² is H or C₁-C₆ alkyl or C₃-C₁₀ cycloalkyl each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen, C₁-C₆ alkyl, C₁-C₆ alkoxy or C₃-C₁₀ cycloalkyl each optionally substituted; or R⁵ and R⁶ are taken together with the atoms to which they are attached to form an optionally substituted phenyl ring;
R⁷ and R⁸ are each independently H, halogen or C₁-C₆ alkyl or C₃-C₁₀ cycloalkyl each group optionally substituted; or
a stereoisomer, tautomer or pharmaceutically acceptable salt thereof;
provided that if R² is H or R³ and R⁴ are taken together to form a tricyclic aromatic ring system, then n is not 2.

2. The compound of claim 1, wherein n is 1 or 2.

3. The compound of claim 1, wherein X is (CR⁷R⁸)ₘ.

4. The compound of claim 3, wherein m is 0.

5. The compound of claim 3, wherein m is 1 and R⁷ and R⁸ are both H.

6. The compound of claim 1, wherein R³ and R⁴ are taken together with the atom to which they are attached to form the structure of formula IA: 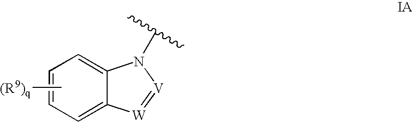
wherein,
q is 0, 1, 2 or 3;
V and W are independently N or CR¹⁰;
each R⁹ is independently halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-7 membered heteroaryl or heterocyclyl group, or C₃-C₆ cycloalkyl, wherein each C₁-C₆ alkyl, C₁-C₆ acyl, C₁-C₆ alkoxy, C₆-C₁₀ aryl, 5-7 membered heteroaryl or heterocyclyl group, or C₃-C₆ cycloalkyl is substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, a 5-7 membered heterocyclyl or heteroaryl ring, —N(R^{a}), —C(O)R^{b}, —OR^{c} and —S(O)ₚR^{d};
R¹⁰ is independently H, halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-7 membered heteroaryl or heterocyclyl group, or C₃-C₆ cycloalkyl, wherein each C₁-C₆ alkyl, C₁-C₆ acyl, C₁-C₆ alkoxy, C₆-C₁₀ aryl, 5-7 membered heteroaryl or heterocyclyl group, or C₃-C₆ cycloalkyl is substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, a 5-7 membered heterocyclyl or heteroaryl ring, —N(R^{a}), —C(O)R^{b}, —OR^{c} and —S(O)ₚR^{d};
each R^{a} is independently H, C₁-C₄ alkyl, —CHO, —C(O)(C₁-C₄ alkyl) or —CO₂(C₁-C₄ alkyl);
each R^{b} is independently H, —OH, —O(C₁-C₄), C₁-C₄ alkyl, —NH₂, —NH(C₁-C₄ alkyl) or —N(C₁-C₄ alkyl)₂;
each R^{c} is independently H, C₁-C₄ alkyl, C₁-C₄ haloalkyl, —CHO or —C(O)(C₁-C₄ alkyl);
each R^{d} is independently H, C₁-C₄ alkyl or —OH; and
each p is independently 0, 1 or 2.

7. The compound of claim 6, wherein q is 0.

8. The compound of claim 6, wherein the compound has the formula IA and W is N and V is CR¹⁰.

9. The compound of claim 8, wherein R¹⁰ is H or methyl.

10. The compound of claim 6, wherein V is N and W is CR¹⁰.

11. The compound of claim 1, wherein R³ and R⁴ are taken together with the atom to which they are attached to form the structure of formula IB: 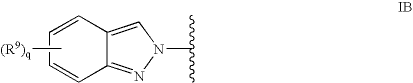
wherein,
q is 0, 1, 2 or 3;
each R⁹ is independently halo, nitro, cyano, hydroxy, S(O)ₚR^{d}, —N(R^{a})ₜ, C₁-C₆ alkyl, C₁-C₆ acyl, C₁-C₆ alkoxy, C₆-C₁₀ aryl, a 5-7 membered heteroaryl or heterocyclyl group, or C₃-C₆ cycloalkyl, wherein each C₁-C₆ alkyl, C₁-C₆ acyl, C₁-C₆ alkoxy, C₆-C₁₀ aryl, 5-7 membered heteroaryl or heterocyclyl group, or C₃-C₆ cycloalkyl is substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, a 5-7 membered heterocyclyl or heteroaryl ring, —N(R^{a})ₜ, —C(O)R^{b}, —OR^{c} and —S(O)ₚR^{d};
each R^{a} is independently H, C₁-C₄ alkyl, —CHO, —C(O)(C₁-C₄ alkyl) or —CO₂(C₁-C₄ alkyl);
each R^{b} is independently H, —OH, —O(C₁-C₄), C₁-C₄ alkyl, —NH₂, —NH(C₁-C₄ alkyl) or —N(C₁-C₄ alkyl)₂;
each R^{c} is independently H, C₁-C₄ alkyl, C₁-C₄ haloalkyl, —CHO or —C(O)(C₁-C₄ alkyl);
each R^{d} is independently H, C₁-C₄ alkyl or —OH; and
each p is independently 0, 1 or 2.

12. The compound of claim 1, wherein R² is methyl or ethyl.

13. The compound of claim 1, wherein R³ and R⁴ are taken together with the atom to which they are attached to form an optionally substituted pyrazole, benzimidazole, indazole or indole ring system.

14. The compound of claim 1, wherein R¹ is C₁-C₆ alkyl or C₃-C₁₀ cycloalkyl.

15. The compound of claim 1, wherein R¹ is methyl, ethyl, propyl, isopropyl, cyclopropylmethyl, cyclopentylmethyl, cyclohexylmethyl, cyclobutyl, cyclopentyl, tetrahydropyran-4-yl, bicyclo[2.2.1]hept-2-yl, or adamantan-2-yl.

16. A compound having the formula: 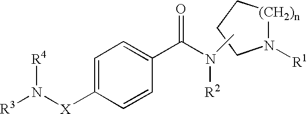
wherein
X is (CH₂)ₘ;
m is 0 or 1;
n is 1 or 2;
R¹ is C₁-C₆ alkyl or C₃-C₆ cycloalkyl each group optionally substituted;
R² is C₁-C₆ alkyl; and
R³ and R⁴ are taken together with the atom to which they are attached to form an optionally substituted monocyclic 5-membered aromatic ring system optionally containing one or two additional heteroatoms selected from N, O or S or an optionally substituted fused bicyclic aromatic ring system optionally containing one to three additional heteroatoms selected from N, O or S; or
a stereoisomer, tautomer or pharmaceutically acceptable salt thereof.

17. The compound of claim 1 selected from the group consisting essentially of:
N-[(3R)-1-isobutylpyrrolidin-3-yl]-N-methyl-4-(2-methyl-1H-benzimidazol-1-yl)benzamide;
N-[(3R)-1-cyclohexylpyrrolidin-3-yl]-N-methyl-4-(2-methyl-1H-benzimidazol-1-yl)benzamide;
N-[(3R)-1-ethylpyrrolidin-3-yl]-N-methyl-4-(2-methyl-1H-benzimidazol-1-yl)benzamide;
N-methyl-4-(2-methyl-1H-benzimidazol-1-yl)-N-[(3R)-1-propylpyrrolidin-3-yl]benzamide;
N-[(3R)-1-(cyclopropylmethyl)pyrrolidin-3-yl]-N-methyl-4-(2-methyl-1H-benzimidazol-1-yl)benzamide;
N-[(3R)-1-(cyclopentylmethyl)pyrrolidin-3-yl]-N-methyl-4-(2-methyl-1H-benzimidazol-1-yl)benzamide;
N-[(3R)-1-(cyclohexylmethyl)pyrrolidin-3-yl]-N-methyl-4-(2-methyl-1H-benzimidazol-1-yl)benzamide;
N-methyl-4-(2-methyl-1H-benzimidazol-1-yl)-N-[(3R)-1-methylpyrrolidin-3-yl]benzamide;
N-[(3R)-1-isopropylpyrrolidin-3-yl]-N-methyl-4-(2-methyl-1H-benzimidazol-1-yl)benzamide;
N-[(3R)-1-cyclobutylpyrrolidin-3-yl]-N-methyl-4-(2-methyl-1H-benzimidazol-1-yl)benzamide;
N-[(3R)-1-cyclopentylpyrrolidin-3-yl]-N-methyl-4-(2-methyl-1H-benzimidazol-1-yl)benzamide;
N-[(3R)-1-cycloheptylpyrrolidin-3-yl]-N-methyl-4-(2-methyl-1H-benzimidazol-1-yl)benzamide;
N-methyl-4-(2-methyl-1H-benzimidazol-1-yl)-N-[(3R)-1-(tetrahydro-2H-pyran-4-yl)pyrrolidin-3-yl]benzamide;
N-[(3R)-1-bicyclo[2.2.1]hept-2-ylpyrrolidin-3-yl]-N-methyl-4-(2-methyl-1H-benzimidazol-1-yl)benzamide;
N-[(3R)-1-adamantan-2-ylpyrrolidin-3-yl]-N-methyl-4-(2-methyl-1H-benzimidazol-1-yl)benzamide;
N-[(3S)-1-isopropylpyrrolidin-3-yl]-N-methyl-4-(2-methyl-1H-benzimidazol-1-yl)benzamide
N-[(3S)-1-cyclobutylpyrrolidin-3-yl]-N-methyl-4-(2-methyl-1H-benzimidazol-1-yl)benzamide;
N-[(3S)-1-cyclopentylpyrrolidin-3-yl]-N-methyl-4-(2-methyl-1H-benzimidazol-1-yl)benzamide;
N-[(3S)-1-cyclohexylpyrrolidin-3-yl]-N-methyl-4-(2-methyl-1H-benzimidazol-1-yl)benzamide;
N-methyl-4-(2-methyl-1H-benzimidazol-1-yl)-N-[(3S)-1-(3-methylcyclopentyl)pyrrolidin-3-yl]benzamide;
N-methyl-4-(2-methyl-1H-benzimidazol-1-yl)-N-{(3S)-1-[(3R)-3-methylcyclopentyl]pyrrolidin-3-yl}benzamide;
N-methyl-4-(2-methyl-1H-benzimidazol-1-yl)-N-[(3S)-1-(2-methylcyclohexyl)pyrrolidin-3-yl]benzamide;
N-methyl-4-(2-methyl-1H-benzimidazol-1-yl)-N-{(3S)-1-[(3R)-3-methylcyclohexyl]pyrrolidin-3-yl}benzamide;
N-methyl-4-(2-methyl-1H-benzimidazol-1-yl)-N-[(3S)-1-(3-methylcyclohexyl)pyrrolidin-3-yl]benzamide;
N-[(3S)-1-(cyclopropylmethyl)pyrrolidin-3-yl]-N-methyl-4-(2-methyl-1H-benzimidazol-1-yl)benzamide;
N-(1-isopropylpiperidin-4-yl)-N-methyl-4-(2-methyl-1H-benzimidazol-1-yl)benzamide;
N-(1-cyclopentylpiperidin-4-yl)-N-methyl-4-(2-methyl-1H-benzimidazol-1-yl)benzamide;
N-(1-cyclohexylpiperidin-4-yl)-N-methyl-4-(2-methyl-1H-benzimidazol-1-yl)benzamide;
N-(1-cyclobutylpiperidin-4-yl)-N-methyl-4-(2-methyl-1H-benzimidazol-1-yl)benzamide;
N-[(3R)-1-isopropylpyrrolidin-3-yl]-N-methyl-4-[(2-methyl-1H-benzimidazol-1-yl)methyl]benzamide;
N-[(3R)-1-cyclobutylpyrrolidin-3-yl]-N-methyl-4-[(2-methyl-1H-benzimidazol-1-yl)methyl]benzamide;
N-[(3R)-1-cyclopentylpyrrolidin-3-yl]-N-methyl-4-[(2-methyl-1H-benzimidazol-1-yl)methyl]benzamide;
N-[(3R)-1-cyclohexylpyrrolidin-3-yl]-N-methyl-4-[(2-methyl-1H-benzimidazol-1-yl)methyl]benzamide;
4-(1H-benzimidazol-1-ylmethyl)-N-[(3R)-1-isopropylpyrrolidin-3-yl]-N-methylbenzamide;
4-(1H-benzimidazol-1-ylmethyl)-N-[(3R)-1-cyclobutylpyrrolidin-3-yl]-N-methylbenzamide;
4-(1H-benzimidazol-1-ylmethyl)-N-[(3R)-1-cyclopentylpyrrolidin-3-yl]-N-methylbenzamide;
N-(1-isopropylpiperidin-4-yl)-N-methyl-4-[(2-methyl-1H-benzimidazol-1-yl)methyl]benzamide;
N-(1-cyclobutylpiperidin-4-yl)-N-methyl-4-[(2-methyl-1H-benzimidazol-1-yl)methyl]benzamide;
N-(1-cyclopentylpiperidin-4-yl)-N-methyl-4-[(2-methyl-1H-benzimidazol-1-yl)methyl]benzamide;
4-(1H-benzimidazol-1-ylmethyl)-N-(1-isopropylpiperidin-4-yl)-N-methylbenzamide;
4-(1H-benzimidazol-1-ylmethyl)-N-(1-cyclobutylpiperidin-4-yl)-N-methylbenzamide;
4-(1H-benzimidazol-1-ylmethyl)-N-(1-cyclopentylpiperidin-4-yl)-N-methylbenzamide;
4-(5-cyano-2-methyl-1H-benzimidazol-1-yl)-N-[(3R)-1-isopropylpyrrolidin-3-yl]-N-methylbenzamide;
4-(5-cyano-2-methyl-1H-benzimidazol-1-yl)-N-[(3R)-1-cyclobutylpyrrolidin-3-yl]-N-methylbenzamide;
4-(5-cyano-2-methyl-1H-benzimidazol-1-yl)-N-[(3R)-1-cyclopentylpyrrolidin-3-yl]-N-methylbenzamide;
4-(2H-indazol-2-yl)-N-[(3R)-1-isopropylpyrrolidin-3-yl]-N-methylbenzamide;
N-[(3R)-1-cyclobutylpyrrolidin-3-yl]-4-(2H-indazol-2-yl)-N-methylbenzamide;
N-[(3R)-1-cyclopentylpyrrolidin-3-yl]-4-(2H-indazol-2-yl)-N-methylbenzamide;
4-(1H-indazol-1-yl)-N-[(3R)-1-isopropylpyrrolidin-3-yl]-N-methylbenzamide;
N-[(3R)-1-cyclobutylpyrrolidin-3-yl]-4-(1H-indazol-1-yl)-N-methylbenzamide;
N-[(3R)-1-cyclopentylpyrrolidin-3-yl]-4-(1H-indazol-1-yl)-N-methylbenzamide;
4-(2H-indazol-2-yl)-N-(1-isopropylpiperidin-4-yl)-N-methylbenzamide;
N-(1-cyclobutylpiperidin-4-yl)-4-(2H-indazol-2-yl)-N-methylbenzamide;
N-(1-cyclopentylpiperidin-4-yl)-4-(2H-indazol-2-yl)-N-methylbenzamide;
4-(1H-indazol-1-yl)-N-(1-isopropylpiperidin-4-yl)-N-methylbenzamide;
N-(1-cyclobutylpiperidin-4-yl)-4-(1H-indazol-1-yl)-N-methylbenzamide;
N-(1-cyclopentylpiperidin-4-yl)-4-(1H-indazol-1-yl)-N-methylbenzamide;
4-(1H-indazol-1-ylmethyl)-N-[(3R)-1-isopropylpyrrolidin-3-yl]-N-methylbenzamide;
N-[(3R)-1-cyclobutylpyrrolidin-3-yl]-4-(1H-indazol-1-ylmethyl)-N-methylbenzamide;
N-[(3R)-1-cyclopentylpyrrolidin-3-yl]-4-(1H-indazol-1-ylmethyl)-N-methylbenzamide;
4-(1H-indazol-1-ylmethyl)-N-(1-isopropylpiperidin-4-yl)-N-methylbenzamide;
N-(1-cyclobutylpiperidin-4-yl)-4-(1H-indazol-1-ylmethyl)-N-methylbenzamide;
N-(1-cyclopentylpiperidin-4-yl)-4-(1H-indazol-1-ylmethyl)-N-methylbenzamide;
N-[(3R)-1-isopropylpyrrolidin-3-yl]-N-methyl-4-(1H-pyrazol-1-yl)benzamide;
N-[(3R)-1-cyclobutylpyrrolidin-3-yl]-N-methyl-4-(1H-pyrazol-1-yl)benzamide;
N-[(3R)-1-cyclopentylpyrrolidin-3-yl]-N-methyl-4-(1H-pyrazol-1-yl)benzamide;
N-(1-isopropylpiperidin-4-yl)-N-methyl-4-(1H-pyrazol-1-yl)benzamide;
N-(1-cyclobutylpiperidin-4-yl)-N-methyl-4-(1H-pyrazol-1-yl)benzamide;
N-(1-cyclopentylpiperidin-4-yl)-N-methyl-4-(1H-pyrazol-1-yl)benzamide;
N-methyl-N-[(3R)-1-(1-methylethyl)pyrrolidin-3-yl]-4-(1H-pyrazol-1-ylmethyl)benzamide;
N-[(3R)-1-cyclobutylpyrrolidin-3-yl]-N-methyl-4-(1H-pyrazol-1-ylmethyl)benzamide;
N-[(3R)-1-cyclopentylpyrrolidin-3-yl]-N-methyl-4-(1H-pyrazol-1-ylmethyl)benzamide;
N-methyl-N-[1-(1-methylethyl)piperidin-4-yl]-4-(1H-pyrazol-1-ylmethyl)benzamide;
N-(1-cyclobutylpiperidin-4-yl)-N-methyl-4-(1H-pyrazol-1-ylmethyl)benzamide;
N-(1-cyclopentylpiperidin-4-yl)-N-methyl-4-(1H-pyrazol-1-ylmethyl)benzamide;
3-fluoro-N-methyl-4-(2-methyl-1H-benzimidazol-1-yl)-N-[(3R)-1-(1-methylethyl)pyrrolidin-3-yl]benzamide;
N-[(3R)-1-cyclobutylpyrrolidin-3-yl]-3-fluoro-N-methyl-4-(2-methyl-1H-benzimidazol-1-yl)benzamide;
N,3-dimethyl-4-(2-methyl-1H-benzimidazol-1-yl)-N-[(3R)-1-(1-methylethyl)pyrrolidin-3-yl]benzamide;
N-[(3R)-1-cyclobutylpyrrolidin-3-yl]-N,3-dimethyl-4-(2-methyl-1H-benzimidazol-1-yl)benzamide;
3-methoxy-N-methyl-4-(2-methyl-1H-benzimidazol-1-yl)-N-[(3R)-1-(1-methylethyl)pyrrolidin-3-yl]benzamide;
N-[(3R)-1-cyclobutylpyrrolidin-3-yl]-3-methoxy-N-methyl-4-(2-methyl-1H-benzimidazol-1-yl)benzamide;
3-fluoro-N-methyl-4-(2-methyl-1H-benzimidazol-1-yl)-N-[1-(1-methylethyl)piperidin-4-yl]benzamide;
N-(1-cyclopentylpiperidin-4-yl)-3-fluoro-N-methyl-4-(2-methyl-1H-benzimidazol-1-yl)benzamide;
2-chloro-N-methyl-4-(2-methyl-1H-benzimidazol-1-yl)-N-[1-(1-methylethyl)piperidin-4-yl]benzamide;
2-chloro-N-(1-cyclopentylpiperidin-4-yl)-N-methyl-4-(2-methyl-1H-benzimidazol-1-yl)benzamide;
N,3-dimethyl-4-(2-methyl-1H-benzimidazol-1-yl)-N-[1-(1-methylethyl)piperidin-4-yl]benzamide;
N-(1-cyclopentylpiperidin-4-yl)-N,3-dimethyl-4-(2-methyl-1H-benzimidazol-1-yl)benzamide;
3-methoxy-N-methyl-4-(2-methyl-1H-benzimidazol-1-yl)-N-[1-(1-methylethyl)piperidin-4-yl]benzamide;
N-(1-cyclopentylpiperidin-4-yl)-3-methoxy-N-methyl-4-(2-methyl-1H-benzimidazol-1-yl)benzamide;
N-[(3R)-1-isopropylpyrrolidin-3-yl]-4-(2-methyl-1H-benzimidazol-1-yl)benzamide;
N-[(3R)-1-cyclopentylpyrrolidin-3-yl]-4-(2-methyl-1H-benzimidazol-1-yl)benzamide;
N-[(3R)-1-cyclohexylpyrrolidin-3-yl]-4-(2-methyl-1H-benzimidazol-1-yl)benzamide;
N-ethyl-N-[(3R)-1-isopropylpyrrolidin-3-yl]-4-(2-methyl-1H-benzimidazol-1-yl)benzamide;
N-[(3R)-1-cyclobutylpyrrolidin-3-yl]-N-ethyl-4-(2-methyl-1H-benzimidazol-1-yl)benzamide;
N-[(3R)-1-cyclopentylpyrrolidin-3-yl]-N-ethyl-4-(2-methyl-1H-benzimidazol-1-yl)benzamide;
N-[(3R)-1-cyclohexylpyrrolidin-3-yl]-N-ethyl-4-(2-methyl-1H-benzimidazol-1-yl)benzamide;
2-chloro-N-methyl-4-(2-methyl-1H-benzimidazol-1-yl)-N-[(3R)-1-(1-methylethyl)pyrrolidin-3-yl]benzamide;
2-chloro-N-[(3R)-1-cyclobutylpyrrolidin-3-yl]-N-methyl-4-(2-methyl-1H-benzimidazol-1-yl)benzamide;
(R)—N-(1-isopropylpyrrolidin-3-yl)-N-methyl-4-((2-methyl-1H-benzo[d]imidazol-1-yl)methyl)-1-naphthamide;
(R)—N-(1-cyclobutylpyrrolidin-3-yl)-N-methyl-4-((2-methyl-1H-benzo[d]imidazol-1-yl)methyl)-1-naphthamide;
N-(1-cyclopentylpiperidin-4-yl)-N-methyl-4-((2-methyl-1H-benzo[d]imidazol-1-yl)methyl)-1-naphthamide;
(R)—N-(1-cyclobutylpyrrolidin-3-yl)-2-methoxy-N-methyl-4-(2-methyl-1H-benzo[d]imidazol-1-yl)benzamide;
(R)—N-(1-isopropylpyrrolidin-3-yl)-2-methoxy-N-methyl-4-(2-methyl-1H-benzo[d]imidazol-1-yl)benzamide;
N—((R)-1-cyclobutylpyrrolidin-3-yl)-N-methyl-4-(2-methyl-1H-benzo[d]imidazol-1-yl)-3-(trifluoromethyl)benzamide;
N—((R)-1-isopropylpyrrolidin-3-yl)-N-methyl-4-(2-methyl-1H-benzo[d]imidazol-1-yl)-3-(trifluoromethyl)benzamide;
(R)—N-(1-cyclobutylpyrrolidin-3-yl)-N-methyl-4-(2-methyl-1H-benzo[d]imidazol-1-yl)-2-(trifluoromethyl)benzamide;
(R)—N-(1-isopropylpyrrolidin-3-yl)-N-methyl-4-(2-methyl-1H-benzo[d]imidazol-1-yl)-2-(trifluoromethyl)benzamide;
(R)—N-(1-cyclobutylpyrrolidin-3-yl)-N,2-dimethyl-4-(2-methyl-1H-benzo[d]imidazol-1-yl)benzamide;
(R)—N-(1-isopropylpyrrolidin-3-yl)-N,2-dimethyl-4-(2-methyl-1H-benzo[d]imidazol-1-yl)benzamide;
N-(1-cyclopentylpiperidin-4-yl)-2-methoxy-N-methyl-4-(2-methyl-1H-benzo[d]imidazol-1-yl)benzamide;
N-(1-isopropylpiperidin-4-yl)-2-methoxy-N-methyl-4-(2-methyl-1H-benzo[d]imidazol-1-yl)benzamide;
N-(1-cyclopentylpiperidin-4-yl)-N-methyl-4-(2-methyl-1H-benzo[d]imidazol-1-yl)-3-(trifluoromethyl)benzamide;
N-(1-isopropylpiperidin-4-yl)-N-methyl-4-(2-methyl-1H-benzo[d]imidazol-1-yl)-3-(trifluoromethyl)benzamide;
N-(1-cyclopentylpiperidin-4-yl)-N-methyl-4-(2-methyl-1H-benzo[d]imidazol-1-yl)-2-(trifluoromethyl)benzamide;
N-(1-isopropylpiperidin-4-yl)-N-methyl-4-(2-methyl-1H-benzo[d]imidazol-1-yl)-2-(trifluoromethyl)benzamide;
N-(1-cyclopentylpiperidin-4-yl)-N,2-dimethyl-4-(2-methyl-1H-benzo[d]imidazol-1-yl)benzamide;
N-(1-isopropylpiperidin-4-yl)-N,2-dimethyl-4-(2-methyl-1H-benzo[d]imidazol-1-yl)benzamide;
(R)—N-(1-cyclobutylpyrrolidin-3-yl)-N-methyl-4-((2-methyl-1H-benzo[d]imidazol-1-yl)methyl)-1-naphthamide;
(R)—N-(1-isopropylpyrrolidin-3-yl)-N-methyl-4-((2-methyl-1H-benzo[d]imidazol-1-yl)methyl)-1-naphthamide;
N-(1-isopropylpiperidin-4-yl)-N-methyl-4-((2-methyl-1H-benzo[d]imidazol-1-yl)methyl)-1-naphthamide;
N-(1-cyclopentylpiperidin-4-yl)-N-methyl-4-((2-methyl-1H-benzo[d]imidazol-1-yl)methyl)-1-naphthamide;
(R)—N-(1-cyclobutylpyrrolidin-3-yl)-N-methyl-3-(2-methyl-1H-benzo[d]imidazol-1-yl)benzamide;
(R)—N-(1-isopropylpyrrolidin-3-yl)-N-methyl-3-(2-methyl-1H-benzo[d]imidazol-1-yl)benzamide;
N-(1-cyclopentylpiperidin-4-yl)-N-methyl-3-(2-methyl-1H-benzo[d]imidazol-1-yl)benzamide;
N-(1-isopropylpiperidin-4-yl)-N-methyl-3-(2-methyl-1H-benzo[d]imidazol-1-yl)benzamide;
(R)—N-(1-cyclobutylpyrrolidin-3-yl)-4-((4-fluoro-1H-benzo[d]imidazol-1-yl)methyl)-N-methylbenzamide;
(R)-4-((4-fluoro-1H-benzo[d]imidazol-1-yl)methyl)-N-(1-isopropylpyrrolidin-3-yl)-N-methylbenzamide;
N-(1-cyclopentylpiperidin-4-yl)-4-((4-fluoro-1H-benzo[d]imidazol-1-yl)methyl)-N-methylbenzamide;
4-((4-fluoro-1H-benzo[d]imidazol-1-yl)methyl)-N-(1-isopropylpiperidin-4-yl)-N-methylbenzamide;
(R)—N-(1-cyclobutylpyrrolidin-3-yl)-4-((4-fluoro-2-methyl-1H-benzo[d]imidazol-1-yl)methyl)-N-methylbenzamide;
(R)-4-((4-fluoro-2-methyl-1H-benzo[d]imidazol-1-yl)methyl)-N-(1-isopropylpyrrolidin-3-yl)-N-methylbenzamide;
N-(1-cyclopentylpiperidin-4-yl)-4-((4-fluoro-2-methyl-1H-benzo[d]imidazol-1-yl)methyl)-N-methylbenzamide;
4-((4-fluoro-2-methyl-1H-benzo[d]imidazol-1-yl)methyl)-N-(1-isopropylpiperidin-4-yl)-N-methylbenzamide;
(R)—N-(1-cyclobutylpyrrolidin-3-yl)-4-((5-fluoro-1H-benzo[d]imidazol-1-yl)methyl)-N-methylbenzamide;
(R)-4-((5-fluoro-1H-benzo[d]imidazol-1-yl)methyl)-N-(1-isopropylpyrrolidin-3-yl)-N-methylbenzamide;
N-(1-cyclopentylpiperidin-4-yl)-4-((5-fluoro-1H-benzo[d]imidazol-1-yl)methyl)-N-methylbenzamide;
4-((5-fluoro-1H-benzo[d]imidazol-1-yl)methyl)-N-(1-isopropylpiperidin-4-yl)-N-methylbenzamide;
(R)—N-(1-cyclobutylpyrrolidin-3-yl)-4-((5-fluoro-2-methyl-1H-benzo[d]imidazol-1-yl)methyl)-N-methylbenzamide;
(R)-4-((5-fluoro-2-methyl-1H-benzo[d]imidazol-1-yl)methyl)-N-(1-isopropylpyrrolidin-3-yl)-N-methylbenzamide;
N-(1-cyclopentylpiperidin-4-yl)-4-((5-fluoro-2-methyl-1H-benzo[d]imidazol-1-yl)methyl)-N-methylbenzamide;
4-((5-fluoro-2-methyl-1H-benzo[d]imidazol-1-yl)methyl)-N-(1-isopropylpiperidin-4-yl)-N-methylbenzamide;
(R)—N-(1-cyclobutylpyrrolidin-3-yl)-4-((6-fluoro-1H-benzo[d]imidazol-1-yl)methyl)-N-methylbenzamide;
(R)-4-((6-fluoro-1H-benzo[d]imidazol-1-yl)methyl)-N-(1-isopropylpyrrolidin-3-yl)-N-methylbenzamide;
N-(1-cyclopentylpiperidin-4-yl)-4-((6-fluoro-1H-benzo[d]imidazol-1-yl)methyl)-N-methylbenzamide;
N-(1-cyclopentylpiperidin-4-yl)-4-((6-fluoro-1H-benzo[d]imidazol-1-yl)methyl)-N-methylbenzamide;
(R)—N-(1-cyclobutylpyrrolidin-3-yl)-4-((6-fluoro-2-methyl-1H-benzo[d]imidazol-1-yl)methyl)-N-methylbenzamide;
(R)-4-((6-fluoro-2-methyl-1H-benzo[d]imidazol-1-yl)methyl)-N-(1-isopropylpyrrolidin-3-yl)-N-methylbenzamide;
N-(1-cyclopentylpiperidin-4-yl)-4-((6-fluoro-2-methyl-1H-benzo[d]imidazol-1-yl)methyl)-N-methylbenzamide;
4-((6-fluoro-2-methyl-1H-benzo[d]imidazol-1-yl)methyl)-N-(1-isopropylpiperidin-4-yl)-N-methylbenzamide;
(R)—N-(1-cyclobutylpyrrolidin-3-yl)-4-((7-fluoro-1H-benzo[d]imidazol-1-yl)methyl)-N-methylbenzamide;
(R)—N-(1-cyclobutylpyrrolidin-3-yl)-4-((7-fluoro-1H-benzo[d]imidazol-1-yl)methyl)-N-methylbenzamide;
N-(1-cyclopentylpiperidin-4-yl)-4-((7-fluoro-1H-benzo[d]imidazol-1-yl)methyl)-N-methylbenzamide;
4-((7-fluoro-1H-benzo[d]imidazol-1-yl)methyl)-N-(1-isopropylpiperidin-4-yl)-N-methylbenzamide;
(R)—N-(1-cyclobutylpyrrolidin-3-yl)-4-((7-fluoro-2-methyl-1H-benzo[d]imidazol-1-yl)methyl)-N-methylbenzamide;
(R)-4-((7-fluoro-2-methyl-1H-benzo[d]imidazol-1-yl)methyl)-N-(1-isopropylpyrrolidin-3-yl)-N-methylbenzamide;
N-(1-cyclopentylpiperidin-4-yl)-4-((7-fluoro-2-methyl-1H-benzo[d]imidazol-1-yl)methyl)-N-methylbenzamide;
4-((7-fluoro-2-methyl-1H-benzo[d]imidazol-1-yl)methyl)-N-(1-isopropylpiperidin-4-yl)-N-methylbenzamide;
(R)-4-((1H-benzo[d]imidazol-1-yl)methyl)-N-(1-cyclobutylpyrrolidin-3-yl)-2-fluoro-N-methylbenzamide;
(R)-4-((1H-benzo[d]imidazol-1-yl)methyl)-2-fluoro-N-(1-isopropylpyrrolidin-3-yl)-N-methylbenzamide;
4-((1H-benzo[d]imidazol-1-yl)methyl)-N-(1-cyclopentylpiperidin-4-yl)-2-fluoro-N-methylbenzamide;
4-((1H-benzo[d]imidazol-1-yl)methyl)-2-fluoro-N-(1-isopropylpiperidin-4-yl)-N-methylbenzamide;
(R)—N-(1-cyclobutylpyrrolidin-3-yl)-2-fluoro-N-methyl-4-((2-methyl-1H-benzo[d]imidazol-1-yl)methyl)benzamide;
(R)-2-fluoro-N-(1-isopropylpyrrolidin-3-yl)-N-methyl-4-((2-methyl-1H-benzo[d]imidazol-1-yl)methyl)benzamide;
N-(1-cyclopentylpiperidin-4-yl)-2-fluoro-N-methyl-4-((2-methyl-1H-benzo[d]imidazol-1-yl)methyl)benzamide;
2-fluoro-N-(1-isopropylpiperidin-4-yl)-N-methyl-4-((2-methyl-1H-benzo[d]imidazol-1-yl)methyl)benzamide;
(R)-4-((1H-benzo[d]imidazol-1-yl)methyl)-N-(1-cyclobutylpyrrolidin-3-yl)-3-fluoro-N-methylbenzamide;
(R)-4-((1H-benzo[d]imidazol-1-yl)methyl)-3-fluoro-N-(1-isopropylpyrrolidin-3-yl)-N-methylbenzamide;
4-((1H-benzo[d]imidazol-1-yl)methyl)-N-(1-cyclopentylpiperidin-4-yl)-3-fluoro-N-methylbenzamide;
4-((1H-benzo[d]imidazol-1-yl)methyl)-3-fluoro-N-(1-isopropylpiperidin-4-yl)-N-methylbenzamide;
(R)—N-(1-cyclobutylpyrrolidin-3-yl)-3-fluoro-N-methyl-4-((2-methyl-1H-benzo[d]imidazol-1-yl)methyl)benzamide;
(R)-3-fluoro-N-(1-isopropylpyrrolidin-3-yl)-N-methyl-4-((2-methyl-1H-benzo[d]imidazol-1-yl)methyl)benzamide;
N-(1-cyclopentylpiperidin-4-yl)-3-fluoro-N-methyl-4-((2-methyl-1H-benzo[d]imidazol-1-yl)methyl)benzamide;
3-fluoro-N-(1-isopropylpiperidin-4-yl)-N-methyl-4-((2-methyl-1H-benzo[d]imidazol-1-yl)methyl)benzamide;
(R)-4-((1H-benzo[d]imidazol-1-yl)methyl)-N-(1-cyclobutylpyrrolidin-3-yl)-2-methoxy-N-methylbenzamide;
(R)-4-((1H-benzo[d]imidazol-1-yl)methyl)-N-(1-isopropylpyrrolidin-3-yl)-2-methoxy-N-methylbenzamide;
4-((1H-benzo[d]imidazol-1-yl)methyl)-N-(1-cyclopentylpiperidin-4-yl)-2-methoxy-N-methylbenzamide;
4-((1H-benzo[d]imidazol-1-yl)methyl)-N-(1-isopropylpiperidin-4-yl)-2-methoxy-N-methylbenzamide;
(R)—N-(1-cyclobutylpyrrolidin-3-yl)-2-methoxy-N-methyl-4-((2-methyl-1H-benzo[d]imidazol-1-yl)methyl)benzamide;
(R)—N-(1-isopropylpyrrolidin-3-yl)-2-methoxy-N-methyl-4-((2-methyl-1H-benzo[d]imidazol-1-yl)methyl)benzamide;
N-(1-cyclopentylpiperidin-4-yl)-2-methoxy-N-methyl-4-((2-methyl-1H-benzo[d]imidazol-1-yl)methyl)benzamide;
N-(1-isopropylpiperidin-4-yl)-2-methoxy-N-methyl-4-((2-methyl-1H-benzo[d]imidazol-1-yl)methyl)benzamide;
(R)-4-((1H-benzo[d]imidazol-1-yl)methyl)-3-chloro-N-(1-cyclobutylpyrrolidin-3-yl)-N-methylbenzamide;
(R)-4-((1H-benzo[d]imidazol-1-yl)methyl)-3-chloro-N-(1-isopropylpyrrolidin-3-yl)-N-methylbenzamide;
4-((1H-benzo[d]imidazol-1-yl)methyl)-3-chloro-N-(1-cyclopentylpiperidin-4-yl)-N-methylbenzamide;
4-((1H-benzo[d]imidazol-1-yl)methyl)-3-chloro-N-(1-isopropylpiperidin-4-yl)-N-methylbenzamide;
(R)-3-chloro-N-(1-cyclobutylpyrrolidin-3-yl)-N-methyl-4-((2-methyl-1H-benzo[d]imidazol-1-yl)methyl)benzamide;
(R)-3-chloro-N-(1-isopropylpyrrolidin-3-yl)-N-methyl-4-((2-methyl-1H-benzo[d]imidazol-1-yl)methyl)benzamide;
3-chloro-N-(1-cyclopentylpiperidin-4-yl)-N-methyl-4-((2-methyl-1H-benzo[d]imidazol-1-yl)methyl)benzamide;
3-chloro-N-(1-isopropylpiperidin-4-yl)-N-methyl-4-((2-methyl-1H-benzo[d]imidazol-1-yl)methyl)benzamide;
4-((1H-benzo[d]imidazol-1-yl)methyl)-N-(1-isopropylpiperidin-3-yl)-N-methylbenzamide;
4-((1H-benzo[d]imidazol-1-yl)methyl)-N-(1-cyclobutylpiperidin-3-yl)-N-methylbenzamide;
4-((1H-benzo[d]imidazol-1-yl)methyl)-N-(1-cyclopentylpiperidin-3-yl)-N-methylbenzamide;
N-(1-isopropylpiperidin-3-yl)-N-methyl-4-((2-methyl-1H-benzo[d]imidazol-1-yl)methyl)benzamide;
N-(1-cyclobutylpiperidin-3-yl)-N-methyl-4-((2-methyl-1H-benzo[d]imidazol-1-yl)methyl)benzamide;
N-(1-cyclopentylpiperidin-3-yl)-N-methyl-4-((2-methyl-1H-benzo[d]imidazol-1-yl)methyl)benzamide;
4-(1H-benzo[d]imidazol-1-yl)-N-(1-isopropylpiperidin-3-yl)-N-methylbenzamide;
4-(1H-benzo[d]imidazol-1-yl)-N-(1-cyclobutylpiperidin-3-yl)-N-methylbenzamide;
4-(1H-benzo[d]imidazol-1-yl)-N-(1-cyclopentylpiperidin-3-yl)-N-methylbenzamide;
N-(1-isopropylpiperidin-3-yl)-N-methyl-4-(2-methyl-1H-benzo[d]imidazol-1-yl)benzamide;
N-(1-cyclobutylpiperidin-3-yl)-N-methyl-4-(2-methyl-1H-benzo[d]imidazol-1-yl)benzamide;
N-(1-cyclopentylpiperidin-3-yl)-N-methyl-4-(2-methyl-1H-benzo[d]imidazol-1-yl)benzamide;
N-methyl-4-(2-methyl-1H-benzimidazol-1-yl)-N-[(3R)-pyrrolidin-3-yl]benzamide;
N-methyl-4-(2-methyl-1H-benzimidazol-1-yl)-N-[(3S)-pyrrolidin-3-yl]benzamide;
N-methyl-4-(2-methyl-1H-benzimidazol-1-yl)-N-piperidin-4-ylbenzamide;
N-methyl-4-[(2-methyl-1H-benzimidazol-1-yl)methyl]-N-[(3R)-pyrrolidin-3-yl]benzamide;
4-(1H-benzimidazol-1-ylmethyl)-N-methyl-N-[(3R)-pyrrolidin-3-yl]benzamide;
N-methyl-4-[(2-methyl-1H-benzimidazol-1-yl)methyl]-N-piperidin-4-ylbenzamide;
4-(1H-benzimidazol-1-ylmethyl)-N-methyl-N-piperidin-4-ylbenzamide;
4-(5-cyano-2-methyl-1H-benzimidazol-1-yl)-N-methyl-N-[(3R)-pyrrolidin-3-yl]benzamide;
4-(1H-indazol-1-yl)-N-methyl-N-[(3R)-pyrrolidin-3-yl]benzamide;
4-(2H-indazol-2-yl)-N-methyl-N-[(3R)-pyrrolidin-3-yl]benzamide;
4-(2H-indazol-2-yl)-N-methyl-N-piperidin-4-ylbenzamide;
4-(1H-indazol-1-yl)-N-methyl-N-piperidin-4-ylbenzamide;
4-(1H-indazol-1-ylmethyl)-N-methyl-N-[(3R)-pyrrolidin-3-yl]benzamide;
4-(1H-indazol-1-ylmethyl)-N-methyl-N-piperidin-4-ylbenzamide;
N-methyl-4-(1H-pyrazol-1-yl)-N-[(3R)-pyrrolidin-3-yl]benzamide;
N-methyl-N-piperidin-4-yl-4-(1H-pyrazol-1-yl)benzamide;
N-methyl-4-(1H-pyrazol-1-ylmethyl)-N-[(3R)-pyrrolidin-3-yl]benzamide;
N-methyl-N-piperidin-4-yl-4-(1H-pyrazol-1-ylmethyl)benzamide;
4-(2-methyl-1H-benzimidazol-1-yl)-N-[(3R)-pyrrolidin-3-yl]benzamide;
N-ethyl-4-(2-methyl-1H-benzimidazol-1-yl)-N-[(3R)-pyrrolidin-3-yl]benzamide;
(R)—N-(1-cyclobutylpyrrolidin-3-yl)-4-((6-methoxy-1H-benzo[d]imidazol-1-yl)methyl)-N-methylbenzamide;
(R)—N-(1-cyclobutylpyrrolidin-3-yl)-4-((5-methoxy-1H-benzo[d]imidazol-1-yl)methyl)-N-methylbenzamide;
(R)—N-(1-cyclobutylpyrrolidin-3-yl)-N-methyl-4-((5-methyl-1H-benzo[d]imidazol-1-yl)methyl)benzamide;
(R)—N-(1-cyclobutylpyrrolidin-3-yl)-N-methyl-4-((6-methyl-1H-benzo[d]imidazol-1-yl)methyl)benzamide;
(R)—N-(1-isopropylpyrrolidin-3-yl)-N-methyl-4-((6-methyl-1H-benzo[d]imidazol-1-yl)methyl)benzamide;
(R)—N-(1-isopropylpyrrolidin-3-yl)-N-methyl-4-((5-methyl-1H-benzo[d]imidazol-1-yl)methyl)benzamide;
(R)—N-(1-isopropylpyrrolidin-3-yl)-4-((5-methoxy-1H-benzo[d]imidazol-1-yl)methyl)-N-methylbenzamide;
(R)—N-(1-isopropylpyrrolidin-3-yl)-4-((6-methoxy-1H-benzo[d]imidazol-1-yl)methyl)-N-methylbenzamide;
N-(1-isopropylpiperidin-4-yl)-N-methyl-4-((6-methyl-1H-benzo[d]imidazol-1-yl)methyl)benzamide;
N-(1-isopropylpiperidin-4-yl)-4-((6-methoxy-1H-benzo[d]imidazol-1-yl)methyl)-N-methylbenzamide;
N-(1-isopropylpiperidin-4-yl)-N-methyl-4-((5-methyl-1H-benzo[d]imidazol-1-yl)methyl)benzamide;
N-(1-isopropylpiperidin-4-yl)-4-((5-methoxy-1H-benzo[d]imidazol-1-yl)methyl)-N-methylbenzamide;
(R)—N-(1-cyclobutylpyrrolidin-3-yl)-4-((6-methoxy-2-methyl-1H-benzo[d]imidazol-1-yl)methyl)-N-methylbenzamide;
(R)—N-(1-cyclobutylpyrrolidin-3-yl)-4-((2,6-dimethyl-1H-benzo[d]imidazol-1-yl)methyl)-N-methylbenzamide;
(R)—N-(1-cyclobutylpyrrolidin-3-yl)-4-((2,5-dimethyl-1H-benzo[d]imidazol-1-yl)methyl)-N-methylbenzamide;
(R)—N-(1-cyclobutylpyrrolidin-3-yl)-4-((5-methoxy-2-methyl-1H-benzo[d]imidazol-1-yl)methyl)-N-methylbenzamide;
(R)—N-(1-isopropylpyrrolidin-3-yl)-4-((5-methoxy-2-methyl-1H-benzo[d]imidazol-1-yl)methyl)-N-methylbenzamide;
(R)-4-((2,5-dimethyl-1H-benzo[d]imidazol-1-yl)methyl)-N-(1-isopropylpyrrolidin-3-yl)-N-methylbenzamide;
(R)-4-((2,6-dimethyl-1H-benzo[d]imidazol-1-yl)methyl)-N-(1-isopropylpyrrolidin-3-yl)-N-methylbenzamide;
(R)—N-(1-isopropylpyrrolidin-3-yl)-4-((6-methoxy-2-methyl-1H-benzo[d]imidazol-1-yl)methyl)-N-methylbenzamide;
N-(1-isopropylpiperidin-4-yl)-4-((5-methoxy-2-methyl-1H-benzo[d]imidazol-1-yl)methyl)-N-methylbenzamide;
4-((2,5-dimethyl-1H-benzo[d]imidazol-1-yl)methyl)-N-(1-isopropylpiperidin-4-yl)-N-methylbenzamide;
4-((2,6-dimethyl-1H-benzo[d]imidazol-1-yl)methyl)-N-(1-isopropylpiperidin-4-yl)-N-methylbenzamide; and
N-(1-isopropylpiperidin-4-yl)-4-((6-methoxy-2-methyl-1H-benzo[d]imidazol-1-yl)methyl)-N-methylbenzamide;
or a pharmaceutically acceptable salt thereof.

18. A method for the treatment of a cognitive disorder related to or affected by the Histamine-3 (H₃) receptor in a patient in need thereof which comprises providing to said patient a therapeutically effective amount of a compound of formula I 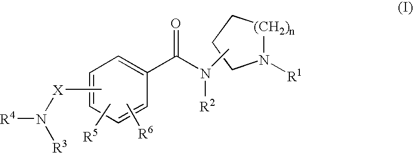
wherein
X is (CR⁷R⁸)ₘ, CO or SO₂;
m is 0 or 1;
n is 1, 2 or 3;
R¹ is C₁-C₆ alkyl, C₁-C₆ haloalkyl, C₃-C₁₀ cycloalkyl or a 3-10 membered cycloheteroalkyl each group optionally substituted;
R² is H or C₁-C₆ alkyl or C₃-C₁₀ cycloalkyl each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen or C₁-C₆ alkyl or C₃-C₁₀ cycloalkyl each optionally substituted; or R⁵ and R⁶ are taken together with the atoms to which they are attached to form an optionally substituted phenyl ring;
R⁷ and R⁸ are each independently H, halogen or C₁-C₆ alkyl or C₃-C₁₀ cycloalkyl each group optionally substituted; or
a stereoisomer, tautomer or pharmaceutically acceptable salt thereof.

19. The method of claim 18 wherein said disorder is a neurodegenerative disorder.

20. The method of claim 19 wherein said disorder is mild cognitive impairment (MCI), dementia, delirium, amnestic disorder, Alzheimer's disease (AD), Parkinson's disease (PD), Huntington's disease (HD), memory disorder, memory deficits associated with depression, schizophrenia, a psychotic disorder, paranoia, mano-depressive illness, attention deficit hyperactivity disorder (ADHD), dyslexia, developmental disorders, Down's syndrome, Fragile X syndrome, loss of executive function, loss of learned information, vascular dementia, cognitive decline, neurodegenerative disorder, HIV-induced dimentia, head trauma, Pick's disease, Creutzfeldt-Jakob disease, Body dementia, vascular dementia, surgical procedure-induced cognitive dysfunction, traumatic brain injury or stroke.

21. The method of claim 20 wherein said disorder is selected from the group consisting of: Alzheimer's disease, attention deficit disorder, schizophrenia; Parkinsons' disease, frontal temporal dementia or depression.

22. A method for the inhibition of an H₃ receptor comprising contacting said receptor with an effective amount of a compound of formula I 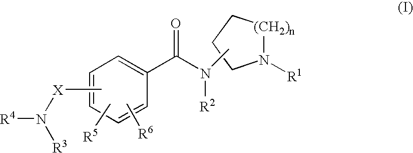
wherein
X is (CR⁷R⁸)ₘ, CO or SO₂;
m is 0 or 1;
n is 1, 2 or 3;
R¹ is H, C₁-C₆ alkyl, C₁-C₆ haloalkyl, cycloalkyl or cycloheteroalkyl each group optionally substituted;
R² is H or C₁-C₆ alkyl or cycloalkyl group each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen or a C₁-C₆ alkyl C₁-C₆ alkoxy or C₃-C₆ cycloalkyl group each optionally substituted; or R⁵ and R⁶ are taken together with the atom to which they are attached to form an optionally substituted phenyl ring;
R⁷ and R⁶ are each independently H, halogen or an alkyl or cycloalkyl group each group optionally substituted; or
a stereoisomer thereof or a pharmaceutically acceptable salt thereof.

23. A pharmaceutical composition which comprises a pharmaceutically acceptable carrier and an effective amount of a compound of formula I: 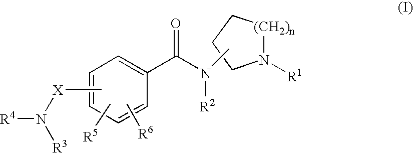
wherein
X is (CR⁷R⁸)ₘ, CO or SO₂;
m is 0 or 1;
n is 1, 2 or 3;
R¹ is H, C₁-C₆ alkyl, C₁-C₆ haloalkyl, C₃-C₁₀ cycloalkyl or a 3-10 membered cycloheteroalkyl each group optionally substituted;
R² is H or C₁-C₆ alkyl or C₃-C₁₀ cycloalkyl each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen or C₁-C₆ alkyl, C₁-C₆ alkoxy or C₃-C₁₀ cycloalkyl each optionally substituted; or R⁵ and R⁶ are taken together with the atoms to which they are attached to form an optionally substituted phenyl ring;
R⁷ and R⁸ are each independently H, halogen or C₁-C₆ alkyl or C₃-C₁₀ cycloalkyl each group optionally substituted; or
a stereoisomer, tautomer or pharmaceutically acceptable salt thereof;
provided that if R² is H or R³ and R⁴ are taken together to form a tricyclic aromatic ring system, then n is not 2.

24. A process for the preparation of a compound of formula I 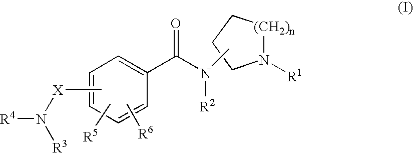
wherein
X is (CR⁷R⁸)ₘ, CO or SO₂;
m is 0 or 1;
n is 1, 2 or 3;
R¹ is H, C₁-C₆ alkyl, C₁-C₆ haloalkyl, C₃-C₁₀ cycloalkyl or a 3-10 membered cycloheteroalkyl each group optionally substituted;
R² is H or C₁-C₆ alkyl or C₃-C₁₀ cycloalkyl each group optionally substituted;
R³ and R⁴ are taken together with the atom to which they are attached to form an optionally substituted monocyclic 5-membered aromatic ring system optionally containing one or two additional heteroatoms selected from N, O or S or an optionally substituted fused bicyclic or tricyclic 9- to 15-membered aromatic ring system optionally containing one to three additional heteroatoms selected from N, O or S; and
R⁵ and R⁶ are each independently H, halogen, C₁-C₆ alkyl, C₁-C₆ alkoxy or C₃-C₁₀ cycloalkyl each optionally substituted; or R⁵ and R⁶ are taken together with the atoms to which they are attached to form an optionally substituted phenyl ring;
R⁷ and R⁸ are each independently H, halogen or C₁-C₆ alkyl or C₃-C₁₀ cycloalkyl each group optionally substituted; or
a stereoisomer, tautomer or pharmaceutically acceptable salt thereof;
which process comprises reacting a compound of formula II 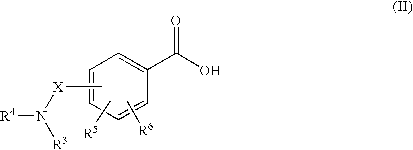
wherein X, R³, R⁴, R⁵ and R⁶ are as described hereinabove for formula I with an azacyclylamine of formula III 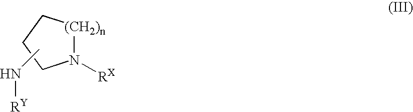
in the presence of a coupling agent and optionally in the presence of a solvent to form a compound of formula IIIa: 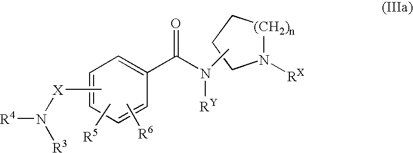
wherein,
R^{X} is R¹ or a protecting group;
R^{Y} is H or C₁-C₆ alkyl or C₃-C₁₀ cycloalkyl each group optionally substituted;
wherein, if R^{Y} is H and R² in the compound of formula I is other than H, than the process further comprises:
reacting activated-R² with the compound of formula IIIa, to form a compound of formula IIIb: 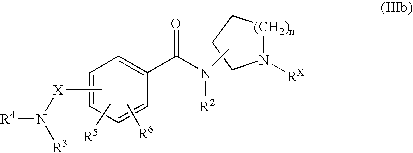
wherein if R^{X} is R¹, then the compound of formula I is formed; or
if R^{X} is a protecting group, then the process further comprises:
deprotecting the compound of formula IIIb to form a deprotected compound; and
if R¹ in the compound of formula I is H, then the compound of formula I is formed; or
if R¹ in the compound of formula I is other than H, then the process further comprises reacting the deprotected compound with activated-R¹;
wherein the compound of formula I is formed.

25. The process of claim 24, wherein:
R^{X} is a protecting group and the protecting group is t-butoxycarbonyl (Boc), benzyl, acetyl, p-methoxybenzyl (PMB), C₁-C₆ alkyl, 9-fluoroenylmethoxycarbonyl (Fmoc), benzyloxycarbonyl (Cbz), trifluoroacetyl, tosyl or trityl;
R^{Y} is H;
activated-R² is halo-R², tosylate-R², R²-anhydride, mesylate-R², or triflate-R²;
activated-R¹ is halo-R¹ or oxo-R¹;
the deprotecting step comprises contacting the compound of formula IIIb with an acid;
activated-R¹ is oxo-R¹ and the reacting the deprotected compound with activated-R¹ step comprises a reductive amination reaction in the presence of a boron-reducing agent;
any of the process steps are performed in a protic solvent, an aprotic solvent, a polar solvent, a nonpolar solvent, a protic polar solvent, an aprotic nonpolar solvent, or an aprotic polar solvent;
any of the process steps includes a purification step comprising at least one of: filtration, extraction, chromatography, trituration, or recrystalization; and/or
any of the process steps includes an analytical step comprising liquid chromatography (LC), mass spectroscopy (MS), liquid chromatography/mass spectroscopy (LC/MS), gas chromatography (GC), gas chromatography/mass spectroscopy (GC/MS), nuclear magnetic resonance (NMR), thin layer chromatography (TLC), melting point (MP) analysis, optical rotation (OR) or elemental analysis.
